SEQUENCE LISTING (105 sequences)

SEQ ID NO: 1 - protein, 83 aa, Artificial Sequence
  Amino acid sequence of the kringle domain of the factor XII
ASCYDGRGLSYRGLARTTLSGAPCQPWASEATYRNVTAEQARNWGLGGHAFCRNPDNDIRPWCFVLNRDRLSWEYCDLAQCQT

SEQ ID NO: 2 - protein, 86 aa, Artificial Sequence
  Amino acid sequence of the kringle domain of the hepatocyte growth factor activator
ERCFLGNGTGYRGVASTSASGLSCLAWNSDLLYQELHVDSVGAAALLGLGPHAYCRNPDNDERPWCYVVKDSALSWEYCRLEACES

SEQ ID NO: 3 - protein, 87 aa, Artificial Sequence
  Amino acid sequence of the kringle domain of the hyaluronan binding protein
DDCYVGDGYSYRGKMNRTVNQHACLYWNSHLLLQENYNMFMEDAETHGIGEHNFCRNPDADEKPWCFIKVTNDKVKWEYCDVSACSA

SEQ ID NO: 4 - protein, 77 aa, Artificial Sequence
  Amino acid sequence of the kringle domain of the neurotrypsin
WGCPAGEPWVSVTDFGAPCLRWAEVPPFLERSPPASWAQLRGQRHNFCRSPDGAGRPWCFYGDARGKVDWGYCDCRH

SEQ ID NO: 5 - protein, 83 aa, Artificial Sequence
  Amino acid sequence of the kringle domain of the retinoic acid-related orphan receptor ROR-1
HKCYNSTGVDYRGTVSVTKSGRQCQPWNSQYPHTHTFTALRFPELNGGHSYCRNPGNQKEAPWCFTLDENFKSDLCDIPACDS

SEQ ID NO: 6 - protein, 82 aa, Artificial Sequence
  Amino acid sequence of the kringle domain of the retinoic acid-related orphan receptor ROR-2
QCYNGSGMDYRGTASTTKSGHQCQPWALQHPHSHHLSSTDFPELGGGHAYCRNPGGQMEGPWCFTQNKNVRMELCDVPSCSP

SEQ ID NO: 7 - protein, 87 aa, Artificial Sequence
  Amino acid sequence of the kringle domain of the kremen protein
PECFTANGADYRGTQNWTALQGGKPCLFWNETFQHPYNTLKYPNGEGGLGEHNYCRNPDGDVSPWCYVAEHEDGVYWKYCEIPACQM

SEQ ID NO: 8 - protein, 81 aa, Artificial Sequence
  Amino acid sequence of the kringle domain of the t-PALP
GGCFWDNGHLYREDQTSPAPGLRCLNWLDAQSGLASAPVSGAGNHSYCRNPDEDPRGPWCYVSGEAGVPEKRPCEDLRCPE

SEQ ID NO: 9 - protein, 93 aa, Artificial Sequence
  Amino acid sequence of the kringle domain of the RGD receptor kinase
LACSHPFSKSATEHVQGHLGKKQVPPDLFQPYIEEICQNLRGDVFQKFIESDKFTRFCQWKNVELNIHLTMNDFSVHRIIGRGGFGEVYGCRK

SEQ ID NO: 10 - protein, 82 aa, Artificial Sequence
  Amino acid sequence of the kringle domain of ApoArgC
QECYHSNGQSYRGTYFTTVTGRTCQAWSSMTPHQHSRTPEKYPNDGLISNYCRNPDGSAGPWCYTTDPNVRWEYCNLTRCSD

SEQ ID NO: 11 - protein, 81 aa, Artificial Sequence
  Amino acid sequence of the kringle domain 1 of the macrophage stimulating protein
RTCIMNNGVGYRGTMATTVGGLPCQAWSHKFPNDHKYTPTLRNGLEENFCRNPDGDPGGPWCYTTDPAVRFQSCGIKSCRE

SEQ ID NO: 12 - protein, 82 aa, Artificial Sequence
  Amino acid sequence of the kringle domain 2 of the macrophage stimulating protein
AACVWGNGEEYRGAVDRTESGRECQRWDLQHPHQHPFEPGKFLDQGLDDNYCRNPDGSERPWCYTTDPQIEREFCDLPRCGS

SEQ ID NO: 13 - protein, 83 aa, Artificial Sequence
  Amino acid sequence of the kringle domain 3 of the macrophage stimulating protein
VSCFRGKGEGYRGTANTTTAGVPCQRWDAQIPHQHRFTPEKYAGKDLRENFCRNPDGSEAPWCFTLRPGMRAAFCYQIRRCTD

SEQ ID NO: 14 - protein, 83 aa, Artificial Sequence
  Amino acid sequence of the kringle domain 4 of the macrophage stimulating protein
QDCYHGAGEQYRGTVSKTRKGVQCQRWSAETPHKPQFTFTSEPHAQLEENFCRNPDGDSHGPWCYTMDPRTPFDYCALRRCAD

SEQ ID NO: 15 - DNA, 252 bp, Artificial Sequence
  Nucleotide sequence encoding the kringle domain of factor XII
gcaagctgctatgatggccgcgggctcagctaccgcggcctggccaggaccacgctctcgggtgcgccctgtcagccgtgggcctcggaggccacctaccggaacgtgactgccgagcaagcgcggaactggggactgggcggccacgccttctgccggaacccggacaacgacatccgcccgtggtgcttcgtgctgaaccgcgaccggctgagctgggagtactgcgacctggcacagtgccagacctag

SEQ ID NO: 16 - DNA, 261 bp, Artificial Sequence
  Nucleotide sequence encoding the kringle domain of the hepatocyte growth factor activator
gagcgctgcttcttggggaacggcactgggtaccgtggcgtggccagcacctcagcctcgggcctcagctgcctggcctggaactccgatctgctctaccaggagctgcacgtggactccgtgggcgccgcggccctgctgggcctgggcccccatgcctactgccggaatccggacaatgacgagaggccctggtgctacgtggtgaaggacagcgcgctctcctgggagtactgccgcctggaggcctgcgaatcctag

SEQ ID NO: 17 - DNA, 264 bp, Artificial Sequence
  Nucleotide sequence encoding the kringle domain of the hyaluronan binding protein
gatgactgctatgttggcgatggctactcttaccgagggaaaatgaataggacagtcaaccagcatgcgtgcctttactggaactcccacctcctcttgcaggagaattacaacatgtttatggaggatgctgaaacccatgggattggggaacacaatttctgcagaaacccagatgcggacgaaaagccctggtgctttattaaagttaccaatgacaaggtgaaatgggaatactgtgatgtctcagcctgctcagcctag

SEQ ID NO: 18 - DNA, 234 bp, Artificial Sequence
  Nucleotide sequence encoding the kringle domain of the neurotrypsin
tggggctgccccgccggcgagccatgggtcagcgtgacggacttcggcgccccgtgtctgcggtgggcggaggtgccacccttcctggagcggtcgcccccagcgagctgggctcagctgcgaggacagcgccacaacttttgtcggagccccgacggcgcgggcagaccctggtgtttctacggagacgcccgtggcaaggtggactggggctactgcgactgcagacactag

SEQ ID NO: 19 - DNA, 252 bp, Artificial Sequence
  Nucleotide sequence encoding the kringle domain of the retinoic acid-related receptor ROR-1
cacaagtgttataacagcacaggtgtggactaccgggggaccgtcagtgtgaccaaatcagggcgccagtgccagccatggaactcccagtatccccacacacacactttcaccgcccttcgtttcccagagctgaatggaggccattcctactgccgcaacccagggaatcaaaaggaagctccctggtgcttcaccttggatgaaaactttaagtctgatctgtgtgacatcccagcttgcgattcatag

SEQ ID NO: 20 - DNA, 252 bp, Artificial Sequence
  Nucleotide sequence encoding the kringle domain of the retinoic acid-related receptor ROR-2
catcagtgctataacggctcaggcatggattacagaggaacggcaagcaccaccaagtcaggccaccagtgccagccgtgggccctgcagcacccccacagccaccacctgtccagcacagacttccctgagcttggaggggggcacgcctactgccggaaccccggaggccagatggagggcccctggtgctttacgcagaataaaaacgtacgcatggaactgtgtgacgtaccctcgtgtagtccctag

SEQ ID NO: 21 - DNA, 264 bp, Artificial Sequence
  Nucleotide sequence encoding the kringle domain of the kremen protein
cccgagtgtttcacagccaatggtgcggattataggggaacacagaactggacagcactacaaggcgggaagccatgtctgttttggaacgagactttccagcatccatacaacactctgaaataccccaacggggaggggggcctgggtgagcacaactattgcagaaatccagatggagacgtgagcccctggtgctatgtggcagagcacgaggatggtgtctactggaagtactgtgagatacctgcttgccagatgtag

SEQ ID NO: 22 - DNA, 246 bp, Artificial Sequence
  Nucleotide sequence encoding the kringle domain of the t-PALP
ggaggctgtttctgggacaacggccacctgtaccgggaggaccagacctcccccgcgccgggcctccgctgcctcaactggctggacgcgcagagcgggctggcctcggcccccgtgtcgggggccggcaatcacagttactgccgaaacccggacgaggacccgcgcgggccctggtgctacgtcagtggcgaggccggcgtccctgagaaacggccttgcgaggacctgcgctgtccagagtag

SEQ ID NO: 23 - DNA, 282 bp, Artificial Sequence
  Nucleotide sequence encoding the kringle domain of the RGD receptor kinase
ctggcctgctcgcatcccttctcgaagagtgccactgagcatgtccaaggccacctggggaagaagcaggtgcctccggatctcttccagccatacatcgaagagatttgtcaaaacctccgaggggacgtgttccagaaattcattgagagcgataagttcacacggttttgccagtggaagaatgtggagctcaacatccacctgaccatgaatgacttcagcgtgcatcgcatcattgggcgcgggggctttggcgaggtctatgggtgccggaagtag

SEQ ID NO: 24 - DNA, 249 bp, Artificial Sequence
  Nucleotide sequence encoding the kringle domain of ApoArgC
caggagtgctaccacagtaatggacagagttatcgaggcacatacttcaccactgtcacaggaagaacctgccaagcttggtcatctatgacgccacaccagcacagtagaaccccagaaaagtacccaaatgatggcttgatctcgaactactgcaggaatccggatggttcggcaggcccttggtgttatacgacggatcccaatgtcaggtgggagtactgcaacctgacacggtgctcagactag

SEQ ID NO: 25 - DNA, 246 bp, Artificial Sequence
  Nucleotide sequence encoding the kringle domain 1 of the macrophage stimulating protein
cggacctgcatcatgaacaatggggttgggtaccggggcaccatggccacgaccgtgggtggcctgccctgccaggcttggagccacaagttcccgaatgatcacaagtacacgcccactctccggaatggcctggaagagaacttctgccgtaaccctgatggcgaccccggaggtccttggtgctacacaacagaccctgctgtgcgcttccagagctgcggcatcaaatcctgccgggagtag

SEQ ID NO: 26 - DNA, 249 bp, Artificial Sequence
  Nucleotide sequence encoding the kringle domain 2 of the macrophage stimulating protein
gccgcgtgtgtctggggcaatggcgaggaataccgcggcgcggtagaccgcacggagtcagggcgcgagtgccagcgctgggatcttcagcacccgcaccagcaccccttcgagccgggcaagttcctcgaccaaggtctggacgacaactattgccggaatcctgacggctccgagcggccatggtgctacactacggatccgcagatcgagcgagagttctgtgacctcccccgctgcgggtcctag

SEQ ID NO: 27 - DNA, 252 bp, Artificial Sequence
  Nucleotide sequence encoding the kringle domain 3 of the macrophage stimulating protein
gtcagctgcttccgcgggaagggtgagggctaccggggcacagccaataccaccactgcgggcgtaccttgccagcgttgggacgcgcaaatcccgcatcagcaccgatttacgccagaaaaatacgcgggcaaagaccttcgggagaacttctgccggaaccccgacggctcagaggcgccctggtgcttcacactgcggcccggcatgcgcgcggccttttgctaccagatccggcgttgtacagactag

SEQ ID NO: 28 - DNA, 252 bp, Artificial Sequence
  Nucleotide sequence encoding the kringle domain 4 of the macrophage stimulating protein
caggactgctaccacggcgcaggggagcagtaccgcggcacggtcagcaagacccgcaagggtgtccagtgccagcgctggtccgctgagacgccgcacaagccgcagttcacgtttacctccgaaccgcatgcacaactggaggagaacttctgccggaacccagatggggatagccatgggccctggtgctacacgatggacccaaggaccccattcgactactgtgccctgcgacgctgcgctgattag

SEQ ID NO: 29 - protein, 87 aa, Artificial Sequence
  Human abrogen (D43)
AKTCYEGNGHFYRGKASTDTMGRPCLPWNSATVLQQTYHAHRSDALQLGLGKHNYCRNPDNRRRPWCYVQVGLKPLVQECMVHDCAD

SEQ ID NO: 30 - protein, 87 aa, Artificial Sequence
  Human derived fusion protein (N43)
AKTCYEGNGHFYRGKASTDTMGRPCLPWNSATVLQQTYHAHRSNALQLGLGKHNYCRNPDNRRRPWCYVQVGLKPLVQECMVHDCAD

SEQ ID NO: 31 - protein, 585 aa, Artificial Sequence [sequence omitted]

SEQ ID NO: 32 - protein, 17 aa, Artificial Sequence
  Human derived linker peptide
DAGGGGSGGGGSGGGGS

SEQ ID NO: 33 - protein, 689 aa, Artificial Sequence
  Human derived fusion protein
ADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLDAGGGGSGGGGSGGGGSKTCYEGNGHFYRGKASTDTMGRPCLPWNSATVLQQTYHAHRSNALQLGLGKHNYCRNPDNRRRPWCYVQVGLKPLVQECMVHDCAD

SEQ ID NO: 34 - protein, 674 aa, Artificial Sequence
  Human derived fusion protein
ADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLDAKTCYEGNGHFYRGKASTDTMGRPCLPWNSATVLQQTYHAHRSNALQLGLGKHNYCRNPDNRRRPWCYVQVGLKPLVQECMVHDCAD

SEQ ID NO: 35 - protein, 672 aa, Artificial Sequence
  Human derived fusion protein
AKTCYEGNGHFYRGKASTDTMGRPCLPWNSATVLQQTYHAHRSNALQLGLGKHNYCRNPDNRRRPWCYVQVGLKPLVQECMVHDCADDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 36 - protein, 15 aa, Artificial Sequence
  Human derived linker peptide
GGGGSGGGGSGGGGS

SEQ ID NO: 37 - protein, 687 aa, Artificial Sequence
  Human derived fusion protein
AKTCYEGNGHFYRGKASTDTMGRPCLPWNSATVLQQTYHAHRSNALQLGLGKHNYCRNPDNRRRPWCYVQVGLKPLVQECMVHDCADGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 38 - protein, 688 aa, Artificial Sequence
  Human derived fusion protein
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLDAGGGGSGGGGSGGGGSKTCYEGNGHFYRGKASTDTMGRPCLPWNSATVLQQTYHAHRSNALQLGLGKHNYCRNPDNRRRPWCYVQVGLKPLVQECMVHDCAD

SEQ ID NO: 39 - protein, 233 aa, Artificial Sequence
  Human derived fusion protein
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 40 - protein, 322 aa, Artificial Sequence
  Human derived fusion protein
ARLEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKKTCYEGNGHFYRGKASTDTMGRPCLPWNSATVLQQTYHAHRSNALQLGLGKHNYCRNPDNRRRPWCYVQVGLKPLVQECMVHDCAD

SEQ ID NO: 41 - protein, 322 aa, Artificial Sequence
  Human derived fusion protein
AKTCYEGNGHFYRGKASTDTMGRPCLPWNSATVLQQTYHAHRSNALQLGLGKHNYCRNPDNRRRPWCYVQVGLKPLVQECMVHDCADRLEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 42 - protein, 85 aa, Artificial Sequence
  Kringle K4 of Plasminogen
HMAQDCYHGDGQSYRGTSSTTTTGKKCQSWSSMTPHRHQKTPENYPNAGLTMNYCRNPDADKGPWCFTTDPSVRWEYCNLKKCSG

SEQ ID NO: 43 - protein, 87 aa, Artificial Sequence
  Kringle K5 of Plasminogen
HMEEDCMFGNGKGYRGKRATTVTGTPCQDWAAQEPHRHSIFTPETNPRAGLEKNYCRNPDGDVGGPWCYTTNPRKLYDYCDVPQCAA

SEQ ID NO: 44 - DNA, 28 bp, Artificial Sequence
  Sense primer for K4 from angiostatin
aaaagcttcatatggcccaggactgcta

SEQ ID NO: 45 - DNA, 27 bp, Artificial Sequence
  Antisense primer for K4 from angiostatin
aaatctagaggatccttatcctgagca

SEQ ID NO: 46 - DNA, 31 bp, Artificial Sequence
  Sense primer for K5 from plasminogen
aacatatggaagaagactgtatgtttgggaa

SEQ ID NO: 47 - DNA, 18 bp, Artificial Sequence
  Antisense primer for K5 from plasminogen
ccggatccttaggccgca

SEQ ID NO: 48 - protein, 132 aa, Artificial Sequence
  Fusion protein - Factor XII
GSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSMGSSHHHHHHSSGLVPRGS

SEQ ID NO: 49 - protein, 85 aa, Artificial Sequence
  Factor XII kringle peptide
GSASCYDGRGLSYRGLARTTLSGAPCQPWASEATYRNVTAEQARNWGLGGHAFCRNPDNDIRPWCFVLNRDRLSWEYCDLAQCQT

SEQ ID NO: 50 - protein, 218 aa, Artificial Sequence
  Fusion protein - HGFA
GSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSMGSSHHHHHHSSGLVPRGSERCFLGNGTGYRGVASTSASGLSCLAWNSDLLYQELHVDSVGAAALLGLGPHAYCRNPDNDERPWCYVVKDSALSWEYCRLEACES

SEQ ID NO: 51 - protein, 88 aa, Artificial Sequence
  HGFA kringle peptide
GSERCFLGNGTGYRGVASTSASGLSCLAWNSDLLYQELHVDSVGAAALLGLGPHAYCRNPDNDERPWCYVVKDSALSWEYCRLEACES

SEQ ID NO: 52 - protein, 219 aa, Artificial Sequence
  Fusion protein - Hyaluronan binding protein
GSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSMGSSHHHHHHSSGLVPRGSDDCYVGDGYSYRGKMNRTVNQHACLYWNSHLLLQENYNMFMEDAETHGIGEHNFCRNPDADEKPWCFIKVTNDKVKWEYCDVSACSA

SEQ ID NO: 53 - protein, 89 aa, Artificial Sequence
  Hyaluronan binding protein kringle peptide
GSDDCYVGDGYSYRGKMNRTVNQHACLYWNSHLLLQENYNMFMEDAETHGIGEHNFCRNPDADEKPWCFIKVTNDKVKWEYCDVSACSA

SEQ ID NO: 54 - protein, 209 aa, Artificial Sequence
  Fusion protein - neurotrypsin
GSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSMGSSHHHHHHSSGLVPRGSWGCPAGEPWVSVTDFGAPCLRWAEVPPFLERSPPASWAQLRGQRHNFCRSPDGAGRPWCFYGDARGKVDWGYCDCRH

SEQ ID NO: 55 - protein, 79 aa, Artificial Sequence
  Neurotrypsin kringle peptide
GSWGCPAGEPWVSVTDFGAPCLRWAEVPPFLERSPPASWAQLRGQRHNFCRSPDGAGRPWCFYGDARGKVDWGYCDCRH

SEQ ID NO: 56 - protein, 215 aa, Artificial Sequence
  Fusion protein - retinoic acid-related receptor ROR-1
GSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSMGSSHHHHHHSSGLVPRGSHKCYNSTGVDYRGTVSVTKSGRQCQPWNSQYPHTHTFTALRFPELNGGHSYCRNPGNQKEAPWCFTLDENFKSDLCDIPACDS

SEQ ID NO: 57 - protein, 85 aa, Artificial Sequence
  Retinoic acid-related receptor ROR-1 kringle peptide
GSHKCYNSTGVDYRGTVSVTKSGRQCQPWNSQYPHTHTFTALRFPELNGGHSYCRNPGNQKEAPWCFTLDENFKSDLCDIPACDS

SEQ ID NO: 58 - protein, 214 aa, Artificial Sequence
  Fusion protein - retinoic acid-related receptor ROR-2
GSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSMGSSHHHHHHSSGLVPRGSQCYNGSGMDYRGTASTTKSGHQCQPWALQHPHSHHLSSTDFPELGGGHAYCRNPGGQMEGPWCFTQNKNVRMELCDVPSCSP

SEQ ID NO: 59 - protein, 84 aa, Artificial Sequence
  Retinoic acid-related orphan receptor ROR-2 kringle peptide
GSQCYNGSGMDYRGTASTTKSGHQCQPWALQHPHSHHLSSTDFPELGGGHAYCRNPGGQMEGPWCFTQNKNVRMELCDVPSCSP

SEQ ID NO: 60 - protein, 219 aa, Artificial Sequence
  Fusion protein - kremen
GSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSMGSSHHHHHHSSGLVPRGSPECFTANGADYRGTQNWTALQGGKPCLFWNETFQHPYNTLKYPNGEGGLGEHNYCRNPDGDVSPWCYVAEHEDGVYWKYCEIPACQM

SEQ ID NO: 61 - protein, 89 aa, Artificial Sequence
  Kremen kringle peptide
GSPECFTANGADYRGTQNWTALQGGKPCLFWNETFQHPYNTLKYPNGEGGLGEHNYCRNPDGDVSPWCYVAEHEDGVYWKYCEIPACQM

SEQ ID NO: 62 - protein, 213 aa, Artificial Sequence
  Fusion protein - t-PALP
GSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSMGSSHHHHHHSSGLVPRGSGGCFWDNGHLYREDQTSPAPGLRCLNWLDAQSGLASAPVSGAGNHSYCRNPDEDPRGPWCYVSGEAGVPEKRPCEDLRCPE

SEQ ID NO: 63 - protein, 83 aa, Artificial Sequence
  t-PALP kringle peptide
GSGGCFWDNGHLYREDQTSPAPGLRCLNWLDAQSGLASAPVSGAGNHSYCRNPDEDPRGPWCYVSGEAGVPEKRPCEDLRCPE

SEQ ID NO: 64 - protein, 225 aa, Artificial Sequence
  Fusion protein - RGD receptor Kinase
GSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSMGSSHHHHHHSSGLVPRGSLACSHPFSKSATEHVQGHLGKKQVPPDLFQPYIEEICQNLRGDVFQKFIESDKFTRFCQWKNVELNIHLTMNDFSVHRIIGRGGFGEVYGCRK

SEQ ID NO: 65 - protein, 95 aa, Artificial Sequence
  RGD receptor Kinase kringle peptide
GSLACSHPFSKSATEHVQGHLGKKQVPPDLFQPYIEEICQNLRGDVFQKFIESDKFTRFCQWKNVELNIHLTMNDFSVHRIIGRGGFGEVYGCRK

SEQ ID NO: 66 - protein, 214 aa, Artificial Sequence
  Fusion protein - ApoArgC
GSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSMGSSHHHHHHSSGLVPRGSQECYHSNGQSYRGTYFTTVTGRTCQAWSSMTPHQHSRTPEKYPNDGLISNYCRNPDGSAGPWCYTTDPNVRWEYCNLTRCSD

SEQ ID NO: 67 - protein, 84 aa, Artificial Sequence
  ApoArgC kringle peptide
GSQECYHSNGQSYRGTYFTTVTGRTCQAWSSMTPHQHSRTPEKYPNDGLISNYCRNPDGSAGPWCYTTDPNVRWEYCNLTRCSD

SEQ ID NO: 68 - protein, 462 aa, Artificial Sequence
  Fusion protein - TrxA kringles 1-4 from the macrophage stimulating protein
GSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSMGSSHHHHHHSSGLVPRGSGSRTCIMNNGVGYRGTMATTVGGLPCQAWSHKFPNDHKYTPTLRNGLEENFCRNPDGDPGGPWCYTTDPAVRFQSCGIKSCREAACVWGNGEEYRGAVDRTESGRECQRWDLQHPHQHPFEPGKFLDQGLDDNYCRNPDGSERPWCYTTDPQIEREFCDLPRCGSVSCFRGKGEGYRGTANTTTAGVPCQRWDAQIPHQHRFTPEKYAGKDLRENFCRNPDGSEAPWCFTLRPGMRAAFCYQIRRCTDQDCYHGAGEQYRGTVSKTRKGVQCQRWSAETPHKPQFTFTSEPHAQLEENFCRNPDGDSHGPWCYTMDPRTPFDYCALRRCA

SEQ ID NO: 69 - protein, 331 aa, Artificial Sequence
  Abrogen kringles 1-4 from the macrophage stimulating protein
GSRTCIMNNGVGYRGTMATTVGGLPCQAWSHKFPNDHKYTPTLRNGLEENFCRNPDGDPGGPWCYTTDPAVRFQSCGIKSCREAACVWGNGEEYRGAVDRTESGRECQRWDLQHPHQHPFEPGKFLDQGLDDNYCRNPDGSERPWCYTTDPQIEREFCDLPRCGSVSCFRGKGEGYRGTANTTTAGVPCQRWDAQIPHQHRFTPEKYAGKDLRENFCRNPDGSEAPWCFTLRPGMRAAFCYQIRRCTDQDCYHGAGEQYRGTVSKTRKGVQCQRWSAETPHKPQFTFTSEPHAQLEENFCRNPDGDSHGPWCYTMDPRTPFDYCALRRCAD

SEQ ID NO: 70 - protein, 217 aa, Artificial Sequence
  Fusion protein - TrxA-K4 kringle from angiostatin
GSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSMGSSHHHHHHSSGLVPRGSHMAQDCYHGDGQSYRGTSSTTTTGKKCQSWSSMTPHRHQKTPENYPNAGLTMNYCRNPDADKGPWCFTTDPSVRWEYCNLKKCSG

SEQ ID NO: 71 - protein, 87 aa, Artificial Sequence
  K4 kringle peptide from angiostatin
GSHMAQDCYHGDGQSYRGTSSTTTTGKKCQSWSSMTPHRHQKTPENYPNAGLTMNYCRNPDADKGPWCFTTDPSVRWEYCNLKKCSG

SEQ ID NO: 72 - protein, 219 aa, Artificial Sequence
  Fusion protein - TrxA-K5 kringle from plasminogen
GSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSMGSSHHHHHHSSGLVPRGSHMEEDCMFGNGKGYRGKRATTVTGTPCQDWAAQEPHRHSIFTPETNPRAGLEKNYCRNPDGDVGGPWCYTTNPRKLYDYCDVPQCAA

SEQ ID NO: 73 - protein, 89 aa, Artificial Sequence
  K5 kringle peptide from plasminogen
GSHMEEDCMFGNGKGYRGKRATTVTGTPCQDWAAQEPHRHSIFTPETNPRAGLEKNYCRNPDGDVGGPWCYTTNPRKLYDYCDVPQCAA

SEQ ID NO: 74 - protein, 6 aa, Artificial Sequence
  Thrombin cleavage site
LVPRGS

SEQ ID NO: 75 - protein, 9 aa, Artificial Sequence
  Purification tag
AWRHPQFGG

SEQ ID NO: 76 - protein, 8 aa, Artificial Sequence
  Purification tag
WSHPQFEK

SEQ ID NO: 77 - protein, 86 aa, Artificial Sequence
  Human kringle domain tPA-K2
SDCYFGNGSAYRGTHSLTESGASCLPWNSMILIGKVYTAQNPSAQALGLGKHNYCRNPDGDAKPWCHVLKNRRLTWEYCDVPSCST

SEQ ID NO: 78 - protein, 86 aa, Artificial Sequence
  Human kringle domain tPA-K1
ATCYEDQGISYRGTWSTAESGAECTNWNSSALAQKPYSGRRPDAIRLGLGNHNYCRNPDRDSKPWCYVFKAGKYSSEFCSTPACSE

SEQ ID NO: 79 - protein, 83 aa, Artificial Sequence
  Human kringle domain thrombin-K2
EQCVPDRGQQYQGRLAVTTHGLPCLAWASAQAKALSKHQDFNSAVQLVENFCRNPDGDEEGVWCYVAGKPGDFGYCDLNYCEE

SEQ ID NO: 80 - protein, 83 aa, Artificial Sequence
  Human kringle domain thrombin-K1
GNCAEGLGTNYRGHVNITRSGIECQLWRSRYPHKPEINSTTHPGADLQENFCRNPDSSTTGPWCYTTDPTVRRQECSIPVCGQ

SEQ ID NO: 81 - protein, 83 aa, Artificial Sequence
  Human kringle domain ROR2-K1
HQCYNGSGMDYRGTASTTKSGHQCQPWALQHPHSHHLSSTDFPELGGGHAYCRNPGGQMEGPWCFTQNKNVRMELCDVPSCSP

SEQ ID NO: 82 - protein, 83 aa, Artificial Sequence; identical to SEQ ID NO: 5
  Human kringle domain ROR1-K1

SEQ ID NO: 83 - protein, 81 aa, Artificial Sequence; identical to SEQ ID NO: 8
  Human kringle domain Putative-K1 (Est)

SEQ ID NO: 84 - protein, 84 aa, Artificial Sequence
  Human kringle domain plasminogen-K5
EDCMFGNGKGYRGKRATTVTGTPCQDWAAQEPHRHSIFTPETNPRAGLEKNYCRNPDGDVGGPWCYTTNPRKLYDYCDVPQCAA

SEQ ID NO: 85 - protein, 77 aa, Artificial Sequence; identical to SEQ ID NO: 4
  Human kringle domain Neurotrypsin-K1

SEQ ID NO: 86 - protein, 83 aa, Artificial Sequence; identical to SEQ ID NO: 14
  Human kringle domain MSP-K4

SEQ ID NO: 87 - protein, 83 aa, Artificial Sequence
  Human kringle domain MSP-K3
VSCFRGKGEGYRGTANTTTAGVPCQRWDAQIPHQHRFTPEKYACKDLRENFCRNPDGSEAPWCFTLRPGMRAAFCYQIRRCTD

SEQ ID NO: 88 - protein, 82 aa, Artificial Sequence
  Human kringle domain MSP-K2
AACVWCNGEEYRGAVDRTESGRECQRWDLQHPHQHPFEPGKFLDQGLDDNYCRNPDGSERPWCYTTDPQIEREFCDLPRCGS

SEQ ID NO: 89 - protein, 81 aa, Artificial Sequence; identical to SEQ ID NO: 11
  Human kringle domain MSP-K1

SEQ ID NO: 90 - protein, 87 aa, Artificial Sequence; identical to SEQ ID NO: 3
  Human kringle domain Hyaluronan BP-K1

SEQ ID NO: 91 - protein, 83 aa, Artificial Sequence
  Human kringle domain HGF-K4
QDCYRGNGKNYMGNLSQTRSGLTCSMWDKNMEDLHRHIFWEPDASKLNENYCRNPDDDAHGPWCYTGNPLIPWDYCPISRCEG

SEQ ID NO: 92 - protein, 83 aa, Artificial Sequence
  Human kringle domain HGF-K3
TECIQGQGEGYRGTVNTIWNGIPCQRWDSQYPHEHDMTPENFKCKDLRENYCRNPDGSESPWCFTTDPNIRVGYCSQIPNCDM

SEQ ID NO: 93 - protein, 82 aa, Artificial Sequence
  Human kringle domain HGF-K2
VECMTCNGESYRGLMDHTESGKICQRWDHQTPHRHKFLPERYPDKGFDDNYCRNPDGQPRPWCYTLDPHTRWEYCAIKTCAD

SEQ ID NO: 94 - protein, 83 aa, Artificial Sequence
  Human kringle domain HGF-K1
RNCIIGKGRSYKGTVSITKSGIKCQPWSSMIPHEHSFLPSSYRGKDLQENYCRNPRGEEGGPWCFTSNPEVRYEVCDIPQCSE

SEQ ID NO: 95 - protein, 86 aa, Artificial Sequence; identical to SEQ ID NO: 2
  Human kringle domain HGF activator-K1

SEQ ID NO: 96 - protein, 83 aa, Artificial Sequence; identical to SEQ ID NO: 1
  Human kringle domain Facto XII-K1

SEQ ID NO: 97 - protein, 86 aa, Artificial Sequence
  Human kringle domain ATF-Kringle (Abrogen)
KTCYEGNGHFYRGKASTDTMGRPCLPWNSATVLQQTYHAHRSDALQLGLGKHNYCRNPDNRRRPWCYVQVGLKPLVQECMVHDCAD

SEQ ID NO: 98 - protein, 82 aa, Artificial Sequence
  Human kringle domain ApoArgC-K1
QECYHSNGQSYRGTYFTTVTGRTCQAWSSMTPHQHSRTPEKYPNDGLISNYCRNPDCSAGPWCYTTDPNVRWEYCNLTRCSD

SEQ ID NO: 99 - protein, 82 aa, Artificial Sequence
  Human kringle domain Angiostatin-K4
QDCYHGDGQSYRGTSSTTTTGKKCQSWSSMTPHRHQKTPENYPNAGLTMNYCRNPDADKGPWCFTTDPSVRWEYCNLKKCSG

SEQ ID NO: 100 - protein, 82 aa, Artificial Sequence
  Human kringle domain Angiostatin-K3
YQCLKGTGENYRGNVAVTVSGHTCQHWSAQTPHTHNRTPENFPCKNLDENYCRNPDGKRAPWCHTTNSQVRWEYCKIPSCDS

SEQ ID NO: 101 - protein, 82 aa, Artificial Sequence
  Human kringle domain Angiostatin-K2
EECMHCSGENYDGKISKTMSGLECQAWDSQSPHAHGYIPSKFPNKNLKKNYCRNPDRELRPWCFTTDPNKRWELCDIPRCTT

SEQ ID NO: 102 - protein, 83 aa, Artificial Sequence
  Human kringle domain Angiostatin-K1
SECKTGNGKNYRGTMSKTKNGITCQKWSSTSPHRPRFSPATHPSEGLEENYCRNPDNDPQGPWCYTTDPEKRYDYCDILECEE

SEQ ID NO: 103 - protein, 9 aa, Artificial Sequence
  Human kringle domain Consensus corresponding to position 54-62
NYCRNPDGD

SEQ ID NO: 104 - protein, 6 aa, Artificial Sequence
  Human kringle domain Consensus corresponding to position 65-70
GPWCYT

SEQ ID NO: 105 - protein, 6 aa, Artificial Sequence
  Human kringle domain Consensus corresponding to position 77-82
VRWEYC

(19) United States
(12) Patent Application Publication
(10) Pub. No.: US 2004/0052777 A1
(43) Pub. Date: Mar. 18, 2004

(54) Kringle polypeptides and methods for using them to inhibit angiogenesis

(76) Inventors: Mark Nesbit, Vincennes (FR); Beatrice Cameron, Paris (FR); Francis Blanche, Paris (FR)

Correspondence Address: WILEY, REIN & FIELDING, LLP, ATTN: PATENT ADMINISTRATION, 1776 K. STREET N.W., WASHINGTON, DC, 20006, US

(21) Appl. No.: 10/425,000

(22) Filed: Apr. 29, 2003

Related U.S. Application Data
(63) Continuation-in-part of application No. 10/233,675, filed on Sep. 4, 2002.

(30) Foreign Application Priority Data
Sep. 4, 2002 (WO) PCT/US02/27885

(51) Int. Cl. A61K 38/43; C12N 9/99; A61K 39/00; C07K 14/54

(52) U.S. Cl. 424/94.1; 435/184; 424/185.1; 530/351

(57) ABSTRACT

The present invention relates to kringle polypeptides and polynucleotides encoding kringle polypeptides and their use as therapeutic agents and in methods of identifying agonist compounds. In effect, the kringle polypeptides according to the present invention are particularly useful for inhibiting in vitro and in vivo proliferation, migration and/or invasion of endothelial cells, recruitment of smooth muscle cells, and/or the formation of vasculature in a tissue. The present invention also relates to the use of kringle polypeptides for treating and/or preventing angiogenesis in tumors and inhibiting the growth of tumors. The present invention further relates to a method of modulating angiogenesis in cells affected by an angiogenic-dependent process and inhibiting unwanted or unregulated angiogenesis in an angiogenesis-associated disease. The present invention also concerns a method of production and purification of kringle polypeptides in a soluble and active form.

### RELATED APPLICATIONS

This application is a continuation-in-part of and claims priority to U.S. application Ser. No. 10/233,675, filed Sep. 4, 2002, and claims priority to U.S. provisional application No. 60/316,300, filed Sep. 4, 2001. The entire contents of each of the prior applications are specifically incorporated herein by reference.

### FIELD OF THE INVENTION

The present invention relates to kringle polypeptides and polynucleotides encoding such polypeptides, called abrogens, and their use as therapeutic agents. In effect, the kringle polypeptides according to the present invention are particularly useful for inhibiting in vitro and in vivo proliferation, migration and/or invasion of endothelial cells, recruitment of smooth muscle cells, and/or the formation of vasculature in a tissue. The present invention also relates to the use of kringle polypeptides for treating and/or preventing angiogenesis in tumors and inhibiting the growth of tumors. The present invention further relates to a method of modulating angiogenesis in cells affected by an angiogenic-dependent process and inhibiting unwanted or unregulated angiogenesis in an angiogenesis-associated disease. The present invention further relates to the identification of agonist compounds useful in modifying cell characteristics and in therapy. The present invention also concerns a method of production and purification of kringle polypeptides in a soluble and active form.

### BACKGROUND OF AND INTRODUCTION TO THE INVENTION AND ITS USES

Angiogenesis is the biological process of generating new blood vessels in a tissue or organ. Under normal physiological conditions, humans or animals undergo angiogenesis only in very specific, restricted situations. For example, angiogenesis is normally observed in wound healing, fetal and and embryonic development and formation of the corpus luteum, endometrium and placenta. It has been reported that new vessel growth is tightly controlled by many angiogenic regulators (Folkman, J., Nature Med., 1: 27-31, 1995) and the switch to an angiogenesic phenotype is controlled through the net balance between up-regulation of angiogenic stimulators and down-regulation of angiogenic suppressors. As the following discussion shows, the polypeptides, agonists, and methods that are described here and are related to the modification of cell proliferation, migration and/or invasion can be useful in research and development and the use of therapeutic agents for a variety of disease conditions. Similarly, methods to identify agonist compounds using the kringle polypeptides of the invention can also be important to a number of disease conditions.

Outgrowth of new blood vessels under pathological conditions has been shown to contribute to pathological conditions, such as diabetic retinopathy, macular degeneration, obesity, rheumatoid arthritis, and psoriasis. Such pathological disease states in which unregulated angiogenesis is present are generally designated as angiogenic-dependent or angiogenic-associated diseases. Anti-angiogenic therapies may thus have important medical benefits in debilitating conditions in which angiogenesis is an important part of the disease process.

Diabetic retinopathy is a potentially blinding, microvascular-related complication of diabetes. This condition is found to be very common in people who have had diabetes for a long time and it results in damage to the fine network of blood vessels in the retina, which can cause decreased vision or even blindness if diabetes is not well controlled. There are two types of diabetic retinopathy that can damage patient sight. One type is designated non-proliferative retinopathy and is characterized by damage to small retinal blood vessels, causing them to leak blood or fluid into the retina. Most visual loss during this stage is due to the fluid accumulating in the central area of the retina, termed the macula, which is required for fine, detailed vision. This accumulation of fluid is called macular edema, and can cause temporarily or permanently decreased vision. The second category of diabetic retinopathy is called proliferative diabetic retinopathy. Proliferative retinopathy is the end result of closure of many small retinal blood vessels. The retinal tissue, which depends on the small vessels for nutrition, will no longer work properly. The areas of the retina in which the blood vessels have closed then foster the growth of the abnormal new blood vessels, which are usually weak and grow at the surface of the retina or into the vitreous jelly. Thus, neovascularization can be very damaging as it can cause bleeding in the eye, retinal scar tissue, and diabetic retinal detachments. In effect, vitreous hemorrhages or fluid exudation from the fragile new vessels is usually observed. Also, the vessels often become fibrotic with time, which leads to retinal detachment. Ultimately, as a result of high pressure in the eye, the optic nerve may be affected, thereby causing glaucoma.

Macular degeneration or age-related macular degeneration (AMD) is a progressive, degenerative condition of the central part of the retina. It is in fact the most common cause of visual impairment for people aged 50 or older. The wet AMD is one form of macular degeneration where new blood vessels grow beneath the retina, where they leak fluid and blood and create a large blind spot in the center of the visual field, resulting in a marked disturbance of vision.

Rheumatoid arthritis (RA) is a chronic, systemic, inflammatory disease that chiefly affects the synovial membranes of multiple joints in the body. It is characterized by the inflammation of the membrane lining the joint, which causes pain, stiffness, warmth, redness and swelling. More precisely, the joint lining, the synovium, in RA becomes inflamed and increases greatly in mass, because of hyperplasia of the lining cells. The volume of synovial fluid increases, resulting in joint swelling and pain. Blood-derived cells, including T cells, B cells, macrophages, and plasma cells, infiltrate the sublining of the synovium. The synovium becomes locally invasive at the synovial interface with cartilage and bone, creating an invasive and destructive front, which is termed ‘pannus’, which causes the erosions observed in RA. Progressive destruction of the articular cartilage, subchondral bone, and periarticular soft tissues eventually combine to produce the deformities that are characteristic of longstanding RA. These deformities result in functional deterioration and profound disability in the long term. In particular, the formation of new blood vessels has been suggested to be of importance in the pathogenesis of RA, in that the expansion of synovial tissue necessitates a compensatory increase in the number and density of synovial blood vessels. The arthritic synovium is in fact a very hypoxic environment, which is a potent signal for the generation of new blood vessels.

Psoriasis is a chronic skin disease occurring in approximately 3% of the population worldwide. It is characterized by excessive growth of the epidermal keratinocytes, inflammatory cell accumulation and excessive dermal angiogenesis. Histological studies, including microscopy, have clearly established that alterations in the blood vessel formation of the skin are a prominent feature of psoriasis.

Some scientists have speculated that there is also a possible correlation between obesity and angiogenesis, and thus that obese people have an excessive blood supply to fat deposit cells. It is thus thought that adipose tissue mass can be regulated through the vasculature. Therefore, by reducing this blood supply, the excessive accumulation of fat deposits would be limited. Rupnick, M. A., et al (PNAS, 2002, 99(Aug. 6):10730-10735), inter alia, has discussed how angiogenic inhibitors may work by depriving fat tissue of needed blood vessels, like tumors, and thus prevent obesity or cause dramatic weight loss. Thui, anti-angiogenic agents may be used as a successful strategy for treating obesity.

Anti-angiogenic therapy further represents a promising approach to cancer treatment because most solid tumors cannot grow without the necessary supply of oxygen and nutrients ensured by the formation of new blood vessels. Several studies have produced direct and indirect evidence in proof that both tumor growth and metastasis are angiogenesis-dependent (Brooks et al., Cell, 1994, 79, 1154-1164; Kim K J et al., Nature, 1993, 362, 841-844), and that expansion of the tumor volume requires the induction of new capillary blood vessels. Moreover, metastatic spread of solid tumors depends on the vascularization of the primary mass, indicating that a blockage of tumor angiogenesis will also block tumor metastasis. Tumor cells promote angiogenesis by the secretion of angiogenic factors, in particular basic fibroblast growth factor (bFGF) (Kandel J. et al., Cell, 1991, 66, 1095-1104) and vascular endothelial growth factor (VEGF) (Ferrara et al., Endocr. Rev., 1997, 18: 425). Tumors may produce one or more of these angiogenic peptides that can synergistically stimulate tumor angiogenesis (Mustonen et al., J. Cell Biol., 1995, 129, 865-898). The expression or administration of anti-angiogenic factors should counteract this tumor-induced angiogenesis. By slowing or stopping tumor growth and metastasis, anti-angiogenic agents provide the first “maintenance therapy” for solid tumors. Antiangiogenic factors might also prove effective in preventing disease in patients at high risk for various malignancies. In addition, they might be useful in early-stage cancers to prevent disease recurrence among patients who have undergone surgical resection of the primary tumor.

Various anti-angiogenic agents have been used to treat human angiogenic dependent or angiogenic associated diseases. These anti-angiogenic agents may be broken down into three primary classes, including agents that specifically inhibit newly sprouting vessels, agents that target and destroy existing tumor blood vessels, and agents that are both cytotoxic to tumor cells and endothelial cells. Many angiogenesis inhibitors are effective primarily when the tumor is renewing its blood vessels, a process that can take many months. In fact, the introduction of an angiogenesis inhibitor can upset the delicate balance of molecules that controls blood vessel formation and in turn can actually cause tumor growth.

By way of example, we note the angiogenic inhibitor endostatin, which corresponds to a 20 kDa proteolytic C-terminal fragment of collagen XVIII, and which has been described as specifically inhibiting endothelial cell proliferation and new blood vessels from forming as well as weakening the existing network of blood vessels that feed primary and metastatic tumors. In preclinical studies, Endostatin protein has been shown to consistently shrink primary and metastatic tumors in mice without the development of drug resistance upon repeated administration. The fumagillin analog, TNP-40, has also been described as being capable of inhibiting the proliferation and migration of endothelial cells. Thalidomide, which is putative epoxide metabolite, has been tested in phase II clinical trials and revealed a 50% biological response in humans with metastatic prostate cancer and recurring primary brain cancer, and a 60% biological response in patients with Kaposi's sarcoma. At doses that do not show any toxic effects in animals, 2-methoxyestradiol (2ME), an orally-active, small molecule anti-proliferative agent, inhibits the growth of human breast tumor cells in vivo and also results in a marked decrease in microvessel density associated with tumors.

The applicants have identified novel kringle polypeptides and new properties of said kringles. Other polypeptides have been structurally characterized as comprising the kringle domains, however until now few have been identified as angiogenic inhibitors (Nesbit et al., Cancer Met Rev., 2000, 19(1-2): 45-9). One such molecule is angiostatin, which consists of the first four disulfide-linked kringle structures of plasminogen (O'Reilly et al., Cell, 1994, 79:315-328; O'Reilly et al., Cell, 1997, 88:1-20). It was however showed that only the first three kringle structures exhibit some anti-angiogenic activity, and that this activity is due to the inhibition of the proliferation of endothelial cell. Kringle 4 of the plasminogen was also described as having no effect on endothelial cell or angiogenesis (Cao et al., J. Biol. Chem., 1996, 271 (46): 22461-7; Cao et al., JBC, 1997, 272(36): 22924-8). Another kringle structure within human plasminogen but ouside of angiostatin is kringle 5 of plasminogen (Lu H et al., BBRC, 1999, 258:668-673). Kringles appear to be autonomous structural and folding domains and are found in a varying number of copies in a variety of proteins having different functions, such as, for example, in some serine proteases, plasma proteins, blood clotting and fibrinolytic proteins. They are believed to play a role as binding mediators and in the regulation of proteolytic activity, however, their functional role is not yet known. These domains are structurally characterized by a triple loop, 3-disulfide bridge structures, whose conformation is defined by a number of hydrogen bonds and small pieces of anti-parallel beta-sheet. Other kringle domains such as kringle 1 and 2 domains of prothrombin, which are fragments released from prothrombin by factor Xa cleavage, have been identified as having anti-endothelial cell proliferative activity by Lee TH et al. (JBC, 1998, vol 273, No. 44, pp. 25505-25512; Rhim et al., BBRC, 1998, 252(2): 513-6) using in vitro angiogenesis assay system with bovine capillary endothelial (BCE) cell proliferation or in the chorioallantoic membrane of chick embryos. The prothrombin kringle-1 and -2 domains were however described as having endothelial cell suppression activities, comparable with those of angiostatin, that is restricted to the inhibition of endothelial cell proliferation. The kringle domain of hepatocyte growth factor was also described as acting via the inhibition of endothelial cell proliferation (Xin et al., BBRC, 2000, 277 (1):186-90).

### SUMMARY OF THE INVENTION

The Applicant has now discovered and selected particularly potent kringle polypeptides, which are capable of efficiently inhibiting endothelial cell activation, proliferation, migration and/or invasion. Also, the newly discovered kringle polypeptides are capable of inhibiting endothelial cell proliferation mediated by several different proangiogenic proteins such as bFGF or VEGF, in specific endothelial cell proliferation assays, whereas previously tested anti-angiogenic agents, such as angiostatin, only inhibits bFGF induced proliferation in these assays. They have been named Abrogens, as they are shown to be unexpectedly capable of abrogating formation of tubules and/or the recruitment of smooth muscle cells to organize as pericytes in newly sprouting vessels. The Abrogens retain a very potent anti-angiogenic activity and are particular good therapeutic candidates as they are or can be fragments of physiologically produced proteins and thus are not immunogenic.

The invention thus provides for novel angiogenesis inhibitor polypeptides that comprise a fragment of a mammalian or human kringle-containing protein including any one of factor XII, hepatocyte growth factor activator (HGFA), hyaluronan binding protein, neurotrypsin, retinoic acid-related receptors 1 and 2 (ROR-1 and ROR-2), the kremen protein, tissue-type plasminogen activator protease (t-PALP), apolipoprotein ArgC, and macrophage stimulating proteins (MSP). These kringle poloypeptides have not been previously identified as separate molecules and/or have not been associated with useful angi-angiogenic activity.

The potent anti-angiogenic activity of the Abrogens was unexpected as the kringle structures they comprise can be found in proteins having a variety of disparate functions or even unknown functions, including functions that could not lead to a prediction of their potency as anti-angiogenic agents. It is known, for example, that the coagulation factor XII is a serum glycoprotein that participates in the initiation of the intrinsic blood coagulation pathway and fibrinolysis. The cloning and sequences of factor XII are described in the international publication WO 00/54787 and by Cool et al. (J Biol Chem. 1985 Nov 5;260(25):13666-76). Deficiency of factor XII is an inherited disorder and has been described as provoking blood coagulation. The plasma factor XII consists of 596 amino acid residues and contains an epidermal growth factor-like region, a kringle region, and a fibronectin region. From its N-terminus, the HGFA has a fibronectin type II domain, an EGF domain, a fibronectin type I domain, an EGF domain, a kringle domain, and a serine protease domain (Liu X L et al., Cancer Res Jul. 15, 1996;56(14):3371-9). Recent findings from the group of Holsberger et al., (Comp Biochem Physiol B Biochem Mol Biol 2002 August;132(4):769-77) suggest that the recombinant HGFA precursor can initiate diverse mitogenic, morphogenic and motogenic effects through its substrate hepatocyte growth factor. A novel hyaluronan-binding protein was purified from human plasma by affinity chromatography on hyaluronan-conjugated Sepharose by Choi-Miura NH (J Biochem (Tokyo) 1996 June;119(6):1157-65). The predicted structure of hyaluronan binding protein showed three epidermal growth factor (EGF) domains, a kringle domain and a serine protease domain from its N-terminus. However, the physiological role of the hyaluronan binding protein has not yet been established. As described in WO98/49322, the neurotrypsin is a serine protease 761 amino acids and contains several domains including a serine protease domain, three scavenger receptor cystein-rich domains, and one kringle domain (Gschwend et al., Mol Cell Neurosci 1997;9(3):207-19). Neurotrypsin has been characterized as being predominantly expressed in the brain structures involved in learning and memory and is associated with autosomal recessive nonsyndromic mental retardation (MR). More precisely, the neurotrypsin is located in presynaptic nerve endings, particularly over the presynaptic membrane lining the synaptic cleft, thereby suggesting that neurotrypsin-mediated proteolysis is required for normal synaptic function and providing potential insights into the pathophysiological bases of mental retardation (Molinari et al, Science Nov. 29, 2002;298(5599):1779-81). The retinoic acid-related receptors ROR-1 and ROR-2 are members of the subfamily 1 of nuclear hormone receptors. A potential ligand of the ROR-1 receptor is cholesterol, suggesting that these receptors could play a key role in the regulation of cholesterol homeostasis and thus represents an important drug target in cholesterol-related diseases (Kallen et al., Structure (Camb) 2002 December;10(12):1697-707). The retinoic acid-related receptor ROR-2 exhibits a highly restricted neuronal-specific expression pattern in brain, retina and pineal gland, and a functional ligand has not yet been identified (Stehlin et al. EMBO J. Nov. 1, 2001;20(21):5822-31. So far, the physiological role of the receptors is not well understood. Nakamura et al. (Biochim Biophys Acta Mar. 19, 2001;1518(1-2):63-72) has recently cloned the kremen protein, which is believed to be a type-I transmembrane protein composed of 473 amino acid residues. Kremen has a kringle domain, a WSC domain, and CUB domains in the extracellular region, while the intracellular region has no apparent conserved motif involved in signal transduction. The physiological role of kremen has not yet been established. As described in the international application WO 01/25252, the tissue plasminogen activator like protease (t-PALP) is expressed in activated monocytes and number of other cells and tissues including cerebellum, smooth muscle, resting and PHA-treated T-cells, GM-CSF-treated macrophages, frontal cortex of the brain, breast lymph node, chronic lymphocytic leukemic spleen, and several others. The t-PALP has a high homology with tissue plasminogen, and is thus believed to a role in the fibrinolytic system, resulting in the dissolution of blood clot. Apolipoprotein ArgC (Byrne et al., Arteriosclerosis, Thrombosis, And Vascular Biol. 1995, 15:65-70) has also been noted as a kringle containing protein, which also appears to contain a splice-donor variation that results in a sequence divergence from other homologous gene sequences. The macrophage stimulating protein (MSP) is a plasma protein containing 711 amino acids that is secreted in the liver into the circulation as a pro-MSP. After proteolytic cleavage, MSP becomes biologically active disulfide-linked alpha beta-chain heterodimeric molecule. In addition to stimulation of macrophages, MSP acts on other cell types including epithelial and hematopoietic cells. It has been reported that MSP is a multifunctional factor regulating cell adhesion and motility, growth and survival. MSP mediates its biological activities by activating a transmembrane receptor tyrosine kinase called RON in humans. MSP can protect epithelial cells from apoptosis by activating two independent signals in the P13-K/AKT or the MAPK pathway (Danilkovitch-Miagkova et al., Apoptosis 2001 June;6(3):183-90). As described below, a number of kringle polypeptides can be produced from or derived from the above-noted proteins.

In accordance with one aspect of the present invention, the Applicant has identified novel angiogenesis inhibitor polypeptides that have the ability to inhibit and/or reduce endothelial cell proliferation, migration or invasion induced by bFGF and VEGF. The present invention thus relates to novel angiogenesis inhibitor polypeptides, polynucleotides encoding them, their use in therapy and in identifying agonist compounds useful in therapy, as well as to a method of production and purification of such polypeptides, and methods of inhibiting unwanted or unregulated angiogenesis in a cell or tumor and in angiogenesis-associated disease.

In a first aspect, the present invention provides new angiogenesis inhibitors as recombinant polypeptides, useful for inhibiting endothelial cell proliferation, migration and proliferation during angiogenesis, which comprise a kringle region of a protein selected from the group: factor XII; hepatocyte growth factor activator (HGFA); hyaluronan binding protein; neurotrypsin; retinoic acid-related receptors 1 and 2 (ROR-1 and ROR-2); the kremen protein; tissue-type plasminogen activator protease (t-PALP); apolipoprotein ArgC; and the macrophage stimulating proteins (MSP).

A second aspect of the invention is directed to a pharmaceutical composition comprising an effective amount of at least one angiogenesis inhibitor polypeptide and a suitable carrier. The invention is also directed to the use of angiogenesis inhibitor polypeptides for the preparation of a drug for treating angiogenesis related disease, such as cancer, diabetic retinopathy, macular degeneration, obesity, rheumatoid arthritis, psoriasis, and other diseases.

A third aspect of the invention concerns a method for treating angiogenesis related diseases by administering at least one angiogenesis inhibitor polypeptide according to the present invention. According to this third aspect, the method of treatment further comprises a combined treatment with another therapy. Conventional therapies that can be combined include radiotherapy, chemotherapy, or surgery. Preferably, the method of treatment and/or prevention of angiogenesis related diseases comprises administering one or more angiogenesis inhibitor polypeptides in combination with one of more therapeutic compounds, polypeptides, or proteins.

In a fourth aspect, the present invention encompasses a method of production and purification of kringle polypeptides in a soluble and active form.

### BRIEF DESCRIPTION OF THE FIGURES

FIG. 1: The proliferative response of transduced HUVEC human endothelial cells to human abrogen (hATF-K from prior application U.S. Ser. No. 10/233,675) and mouse abrogen (mATK-K from prior application U.S. Ser. No. 10/233,675). Cultured cells were transduced with adenoviral vectors containing an expression cassette for producing the abrogen polypeptide (hATF-K and mATF-K), a control, CMV promoter only vector (CMV), and the full amino terminal fragment of plasminogen (hATF or mATF). In FIG. 1A, the left axis indicates the degree of cell proliferation and each of the boxes represents the level of cell proliferation under a treatment regimen as indicated by the addition of bFGF, VEGF, or both. The reduction in cell proliferation in all samples where the human abrogen polypeptide is expressed (hATF-K) is markedly reduced compared to controls (CMV, hATF, and mATF). The proliferation in the mouse abrogen expressing cells (mATF-K) is also markedly reduced. FIG. 1B shows representative cell cultures from mouse and human full ATF polypeptides and mouse and human ATF-Kringle containing abrogen polypeptides (see Examples). The first page shows Control (full human ATF treated with FGF) compared to hATF-Kringle containing polypeptide treated with FGF. The remaining pages list the adenoviral vector used to transduce the cells (see Examples).

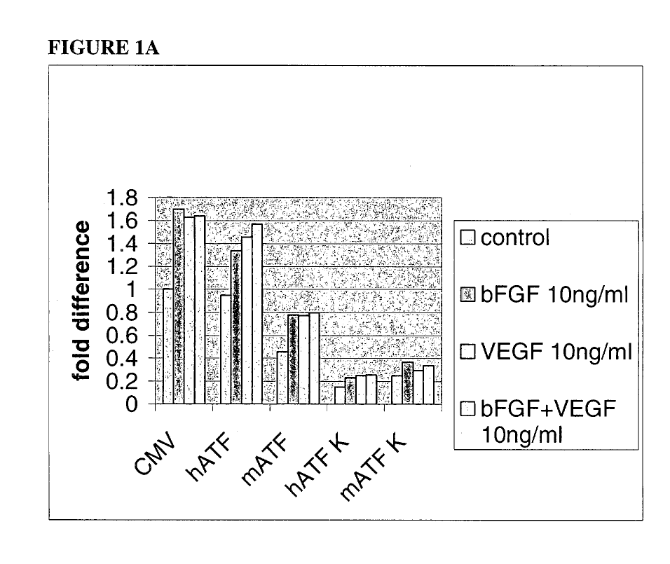

FIG. 2: Exemplary human protein sequences having a kringle domain possessing the consensus region from Asn 53 to Asp 59 of hATF-K and the with the 6 conserved Cys, 2 conserved Trp, and conserved Gly and Arg residues aligned. These proteins and homologs, isoforms, and derivatives of them, can be used in methods of the invention and used to produce kringle polypeptides and polynucleotides of the invention. As noted in the text, additional protein sequences can be selected and additional animal species can be selected.

FIG. 3: Effect of anti-angiogenic polypeptides on tubule growth in endothelial cells. Because culture conditions rapidly deplete anti-angiogenic factors if they are added as a recombinant or purified polypeptide, HUVECs are directly transduced with adenoviral vectors to provide consistent protein expression and secretion for the duration of the assay (7-10 days). HUVECs are transduced with Adenovirus expressing: human abrogen, HATF-K, mouse abrogen, mATF-K, and human endostatin (FIG. 3A) or human Angiostatin (FIG. 3B). Control adenovirus containing the LacZ or no gene of interest (empty control) is also included. The transduced cells are then cultured in a 3-dimensional matrix of fibrin with recombinant VEGF or bFGF added, as indicated. Tubule formation as a marker for activation and proliferation of endothelial cells is then visualized and recorded. Tubule formation in both the bFGF and VEGF treated cells is markedly inhibited in only the abrogen expressing cultures.

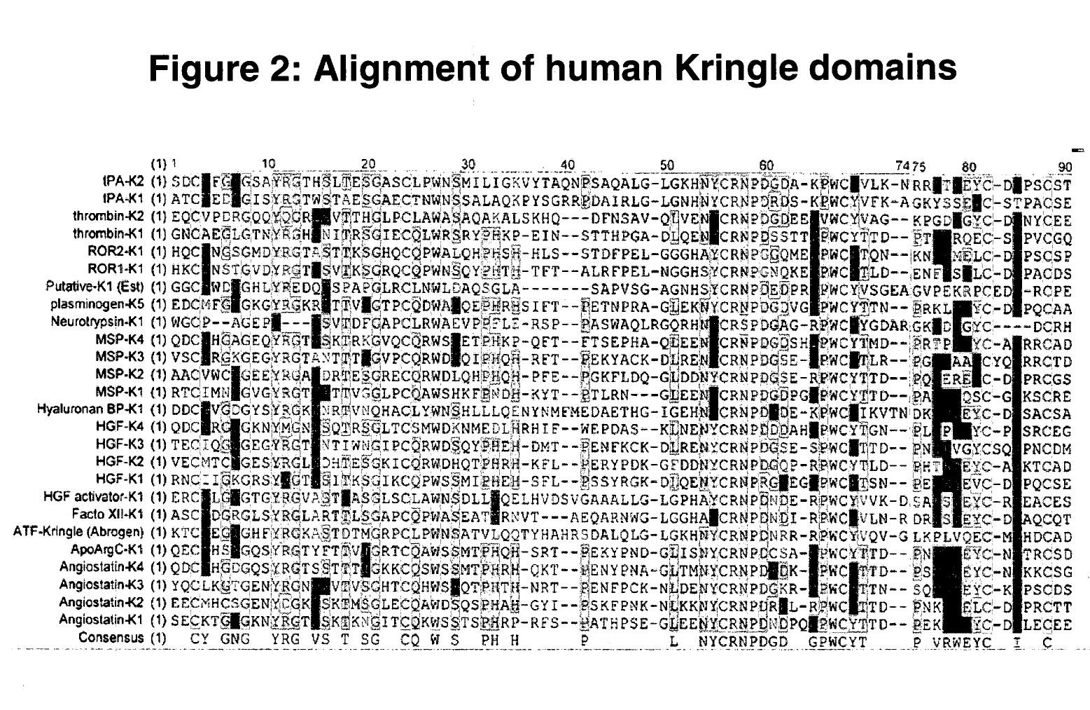

FIG. 4: Prevention of tumor metastasis in mouse 4T1 lung cancer model. Control empty plasmid and abrogen (hATF-K or mATF-K) expression cassette containing plasmid introduced via electrotransfer 6 days prior to injection of 4T1 tumor cells. Approximately 250,000 tumor cells are injected subcutaneously. Fifteen days after injection, primary tumors are removed in a surgical procedure. Lungs are harvested 35 days post tumor injection and the size and number of metastatic tumor colonies measured.

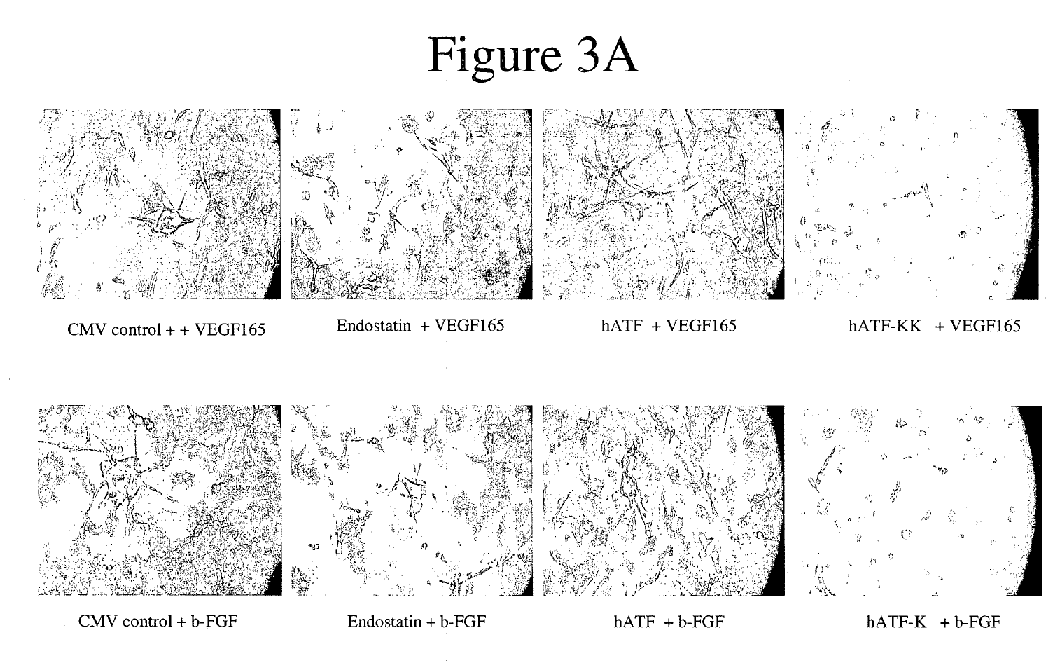

FIG. 5: Prevention of tumor metastasis in mouse 4T1 lung cancer model. Control empty plasmid compared to mATF-K expression plasmid. The assay protocol is the same as in FIG. 4.

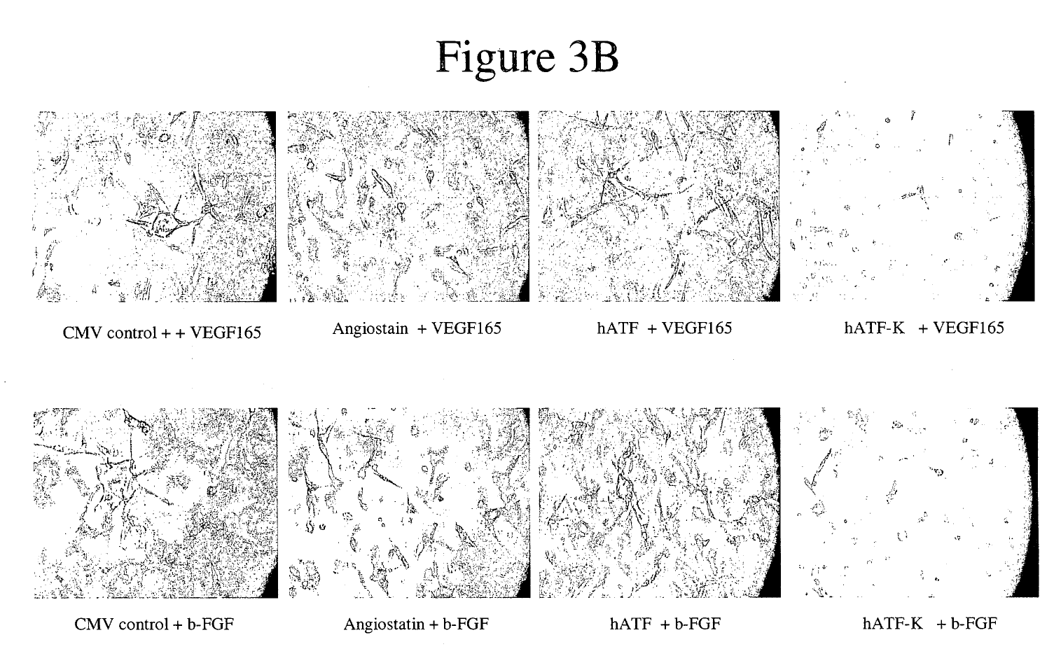

FIG. 6: Prevention of tumor metastasis in mouse 3LL Boston lung cancer model. Control empty plasmid compared to mATF-K expression plasmid. The assay protocol is the same as FIG. 4, with the exception that 3LL Boston cells are used.

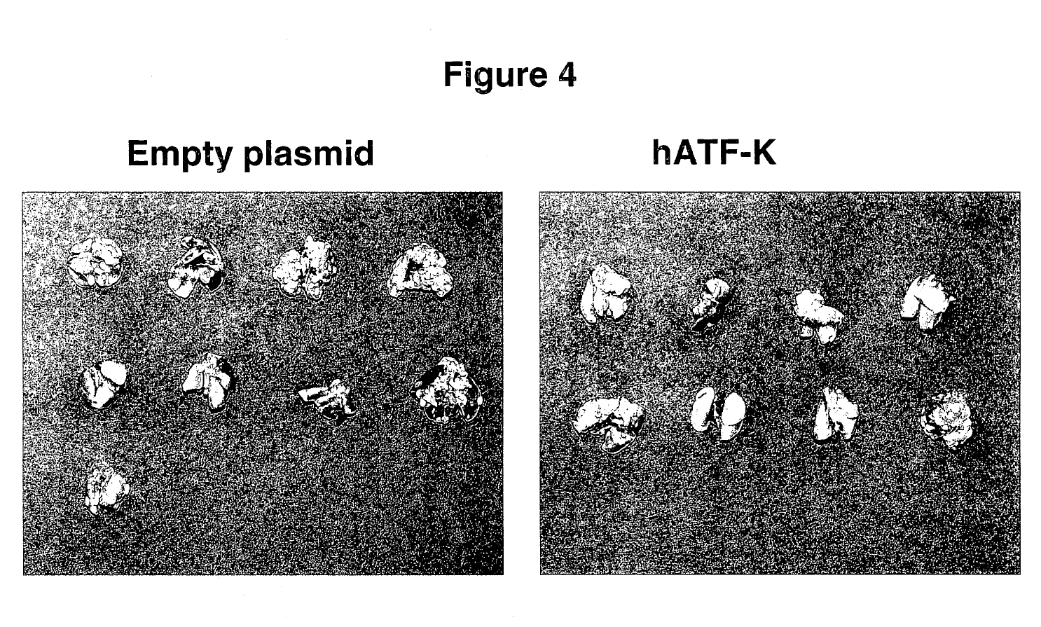

FIG. 7: Prevention of tumor metastasis in mouse 3LL Boston lung cancer model. Control empty plasmid compared to experimental control mEndostatin expression plasmid. The assay protocol is the same as FIG. 6.

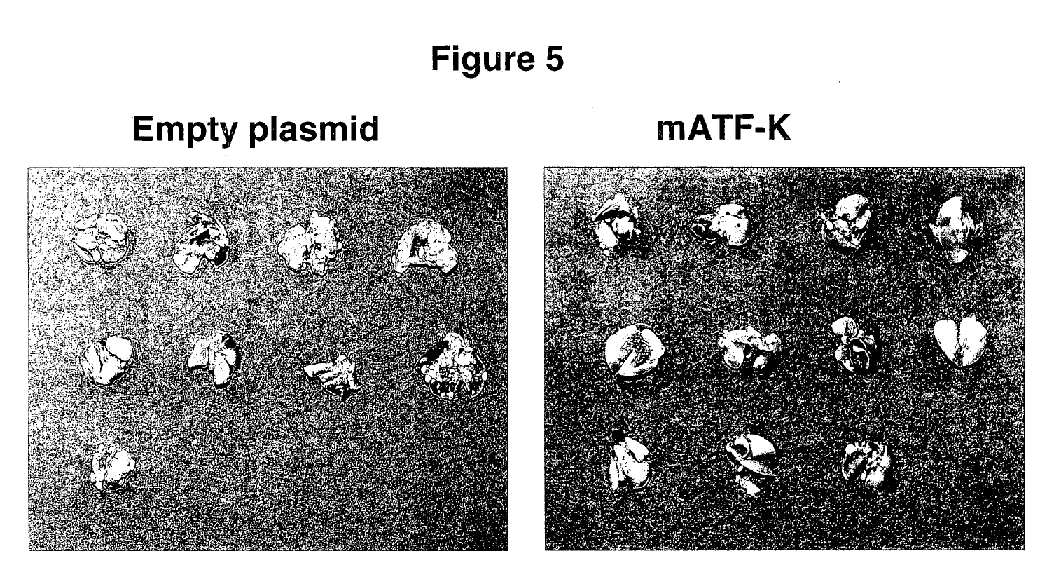

FIG. 8: Measurement of size and number of metastasis in the 4T1 lung tumor model described for FIG. 4. Each spot represents the weight of the lung from each animal surveyed (C57BL/6 mice), indicating the relative size of the tumor nodules present. The left axis indicates the number of visible tumor nodules for each of the animals. With the exception of one animal in the hATF-K sample, the abrogen expressing vector treatment animals show a reduction in both the size and number of metastatic tumor nodules as compared to control. The hATF-K animals with abnormally high number of nodules were not further examined for experimental or procedural error or expression of HATF-K. Here the controls are empty plasmid (Control) and an alkaline phosphatase expressing control plasmid (mSEAP).

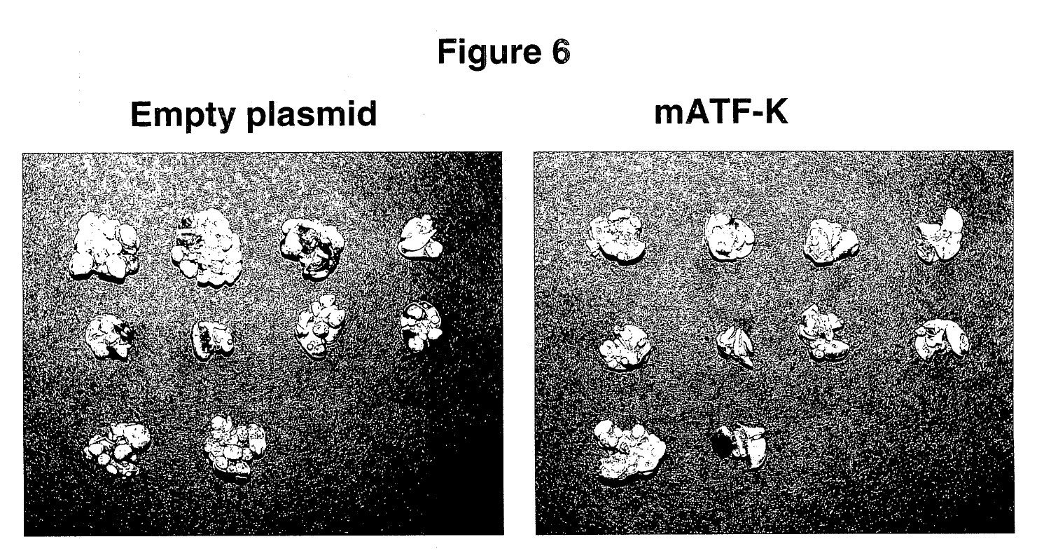

FIG. 9: Measurement of size and number of metastasis in the 3LL Boston lung tumor model described for FIG. 4 using the graphical representation method described for FIG. 7. Controls are the same as in FIG. 7. Again, the use of both the mouse and human abrogen expressing vectors (mATF-K and hATF-K) results in significant reduction in tumor metastasis.

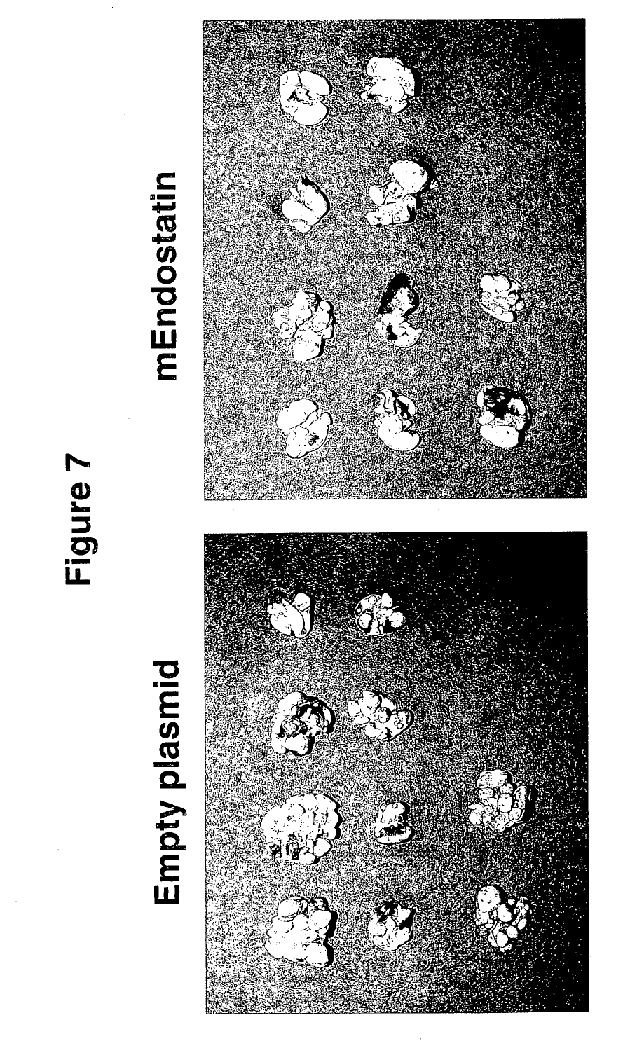

FIG. 10: Measurement of size and number of metastasis in the 3LL Boston lung tumor model as described for FIG. 9. These data indicate that treatment with mouse endostatin or angiostatin, or either mouse or human ATF-K, reduce the number and size of the lung metastatic nodules compared to control treatment. The fact that both mouse and human abrogen encoding vectors are efficacious indicates that the species-specific characteristics that limit the use of the endostatin and angiostatin polypeptides are not present in the abrogen polypeptides. Furthermore, the abrogen polypeptides appear at least as efficacious as the either endostatin or angiostatin and much more efficacious than a combined endostatin/angiostatin treatment (mEndo/mAngio).

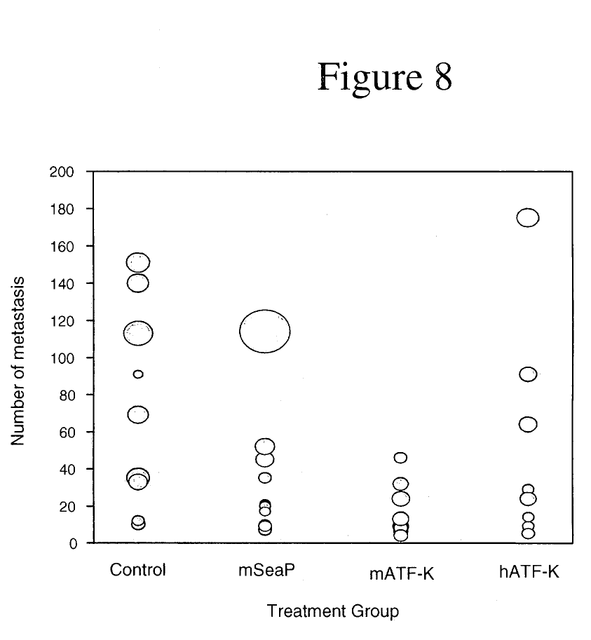

FIG. 11: Systemic expression of mouse or human derived abrogen polypeptides (here listed as MuPAK or HuPAK) from vector introduced into muscle significantly reduces the formation of spontaneous lung metastases in the 3LL-B tumor model. Systemic expression of therapeutic transgenes from the muscle is established 6 days before C57BL/6 mice are injected with a tumorigenic dose of 3LL-B tumor cells. The primary tumor is carefully excised 15 days post cell injection. The study is terminated on day 35 and lung metastases were counted. Panel A: lungs from mice treated with empty expression vector; Panel B: mice treated with human derived ATF-Kringle abrogen expressing vector (HuPAK); and Panel C: with treated with mouse derived ATF-Kringle abrogen expressing vector (MuPAK); Panel D: graphically shows the number and size of metastatic nodules present as the diameter of each “bubble” represents the lung weight.

FIG. 12: Systemic expression of mouse or human abrogen (here listed as MuPAK or HuPAK) from muscle significantly reduces the formation of spontaneous lung metastases in the MDA-MB-435 tumor model. Systemic expression of therapeutic transgenes from the muscle is established 10 days after SCID/bg mice are injected with a tumorigenic dose of MDA-MB-435 (human breast adenocarcinoma tumor cells). The primary tumor is carefully excised when a volume of 250 to 350 mm₃ is reached. The study is terminated on day 89 and lung metastases measured. Panel A: lungs from mice treated with control mSEAP; Panel B: with treated with mouse derived ATF-Kringle abrogen expressing vector (here MuPAK); Panel C: mice treated with human derived ATF-Kringle abrogen expressing vector (HuPAK); and Panel D: graphically shows lung metastases counts as noted above.

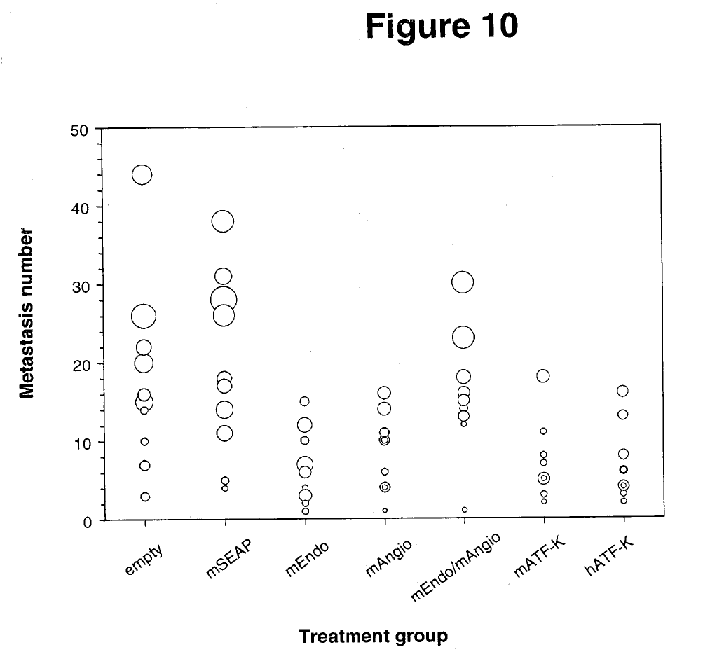

FIG. 13A: is a schematic representation of the plasmid pXL2996.

FIG. 13B: is a schematic representation of the plasmid pMB063.

FIG. 13C is a schematic representation of the plasmid pBA140.

FIG. 14: is a schematic representation of the plasmid pMB060 and fusion construct.

FIG. 15: is a schematic representation of the plasmid pMB059 and fusion construct.

FIG. 16 is a schematic representation of the plasmid pMB056 and fusion construct.

FIG. 17: is a schematic representation of the plasmid pMB055 and fusion construct.

FIG. 18: is a schematic representation of the plasmid pMB060m prepro and fusion construct.

FIG. 19: is a schematic representation of the plasmid pMB053 and fusion construct.

FIG. 20: is a schematic representation of the plasmid pMB057 and fusion construct.

FIG. 21: is a schematic representation of the plasmid pXL4128.

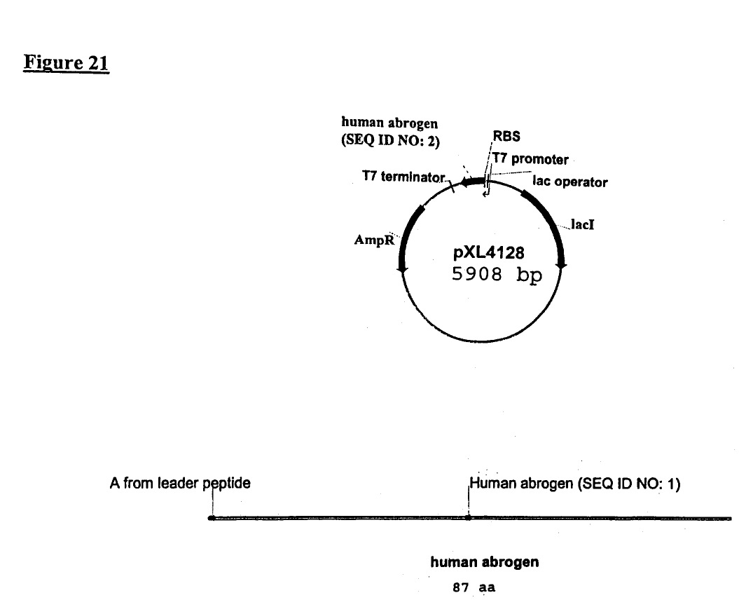

FIG. 22: is a schematic representation of the plasmid pET28-Trx, which can be used for the methods to produce abrogen fusion protein.

FIG. 23: is a schematic representation of plasmids pXL4189 (top) and pXL4215 (bottom).

FIG. 24: is a schematic representation of plasmids pXL4190 (top) and pXL4219 (bottom).

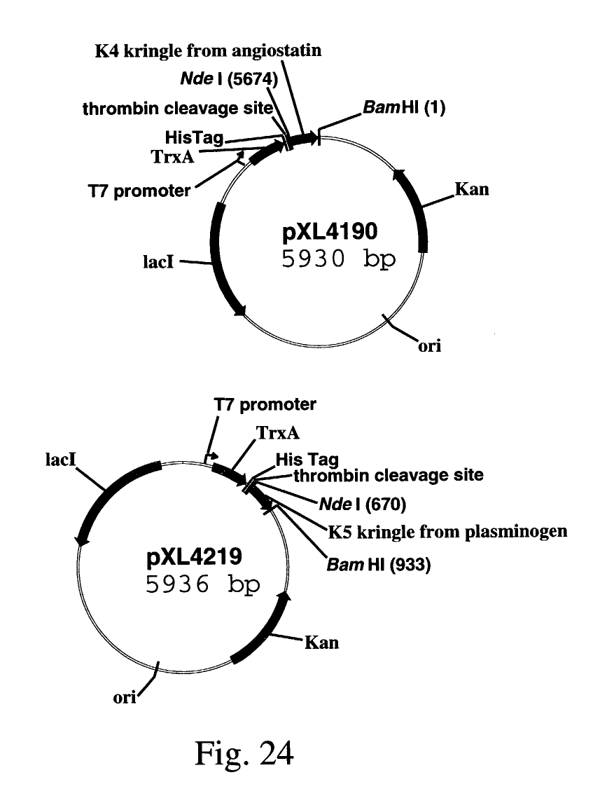

FIG. 25: Production of Fusion Proteins. This Figure shows the expression products from various plasmids separated by gel electrophoresis. The far left lane of the gel image (lane #M) shows the molecular weight markers, indicated by the numbers on the left side (Kda). Lane #2 is the total cell extract from cell expression using pXL4189 (TrxA-abrogen N43 fusion), for expressing abrogen N43. Lane #8 is the soluble fraction from the cell expression of Lane #2. The results show that a substantial percentage of fusion protein is soluble and can be cleaved to produce soluble abrogen N43. Lane #9 is the remaining cell pellet from Lane #2. Lane #5 is the total cell extract from cell expression using pXL4190 (TrxA-K4 angiostatin fusion), for expressing K4 kringle domain from angiostatin. Lane #10 is the soluble fraction from the cell expression of Lane #5. The results show that a substantial percentage of fusion protein is soluble and can be cleaved to produce soluble K4 polypeptide. Lane #11 is the remaining cell pellet from Lane #5.

### DETAILED DESCRIPTION

Throughout this disclosure, the applicant refers to journal articles, patent documents, published references, web pages, sequence information available in databases, and other sources of information. One skilled in the art can use the entire contents of any of the cited sources of information to make and use aspects of this invention. Each and every cited source of information is specifically incorporated herein by reference in its entirety. Portions of these sources may be included in this document as allowed or required. However, the meaning of any term or phrase specifically defined or explained in this disclosure shall not be modified by the content of any of the sources. The description and examples that follow are merely exemplary of the scope of this invention and content of this disclosure. One skilled in the art can devise and construct numerous modifications to the examples listed below without departing from the scope of this invention.

In a first aspect, the invention provides for isolated Abrogens peptides as novel angiogenesis inhibitor polypeptides that comprise a fragment of a mammalian or human kringle-containing protein, which can be selected from the group of proteins consisting of factor XII, hepatocyte growth factor activator (HGFA), hyaluronan binding protein, neurotrypsin, retinoic acid-related receptors 1 and 2 (ROR-1 and ROR-2), the kremen protein, tissue-type plasminogen activator protease (t-PALP), apolipoprotein ArgC, and macrophage stimulating proteins (MSP).

The Abrogens, according to the present invention, are capable of inhibiting tube formation in endothelial cell cultures induced by bFGF and VEGF, and/or capable of reducing cell proliferation induced by bFGF and VEGF, and/or capable of inhibiting the metastasis of mammalian tumors. The novel polypeptides can advantageously be used to effectively inhibit or reduce cell proliferation, migration and/or invasion associated with bFGF and VEGF treatment, and/or inhibiting unwanted or unregulated angiogenesis in a tumor and/or in an angiogenesis-associated disease.

More particularly, the Abrogens according to the invention comprises the amino acid sequence as set forth in SEQ ID NO: 1-14, or where the polypeptides are in a form that does not exist in nature and has not been previously disclosed. A polypeptide according to the present invention includes a polypeptide having an amino acid sequence at least 80% identical, more preferably at least 90% identical, and still more preferably at least 95%, 96%, 97%, 98%, or 99% identical to one of the polypeptides set forth in SEQ ID NO: 1-14.

The Abrogen polypeptides or derivatives can be recombinant polypeptides or purified polypeptides.

The invention also consists an amino acid sequence encoded by the nucleic acid sequence of any one of the sequences set forth in SEQ ID NO: 15-28 as well as a nucleic acid sequence that encodes any one of SEQ ID NO: 1-14. A polynucleotide according to the invention has a nucleic acid sequence at least 80% or 90% identical, and more preferably at least 95%, 96%, 97%, 98%, or 99% identical to any nucleic acid sequences set forth in SEQ ID NO: 15-28 or a nucleic acid encoding an amino acid sequence set forth in SEQ ID NO: 1-14, or a polynucleotide that hybridizes under stringent conditions to any nucleic acid sequence set forth in SEQ ID NO: 15-28 or a nucleic acid sequence encoding an amino acid sequences set forth in SEQ ID NO: 114.

The nucleic acids comprising any of the sequences as set forth SEQ ID NO.: 15-28 or any others of the invention can be DNA, RNA, or DNA or RNA comprising modified nucleotide bases. A nucleic acid encoding one of the Abrogen polypeptides of the present invention can also be operably linked to a variety of or one or more sequences used in expression vectors, and/or cloning vectors, and/or other vectors. For example, the kringle polypeptide encoding nucleic acids can be linked to a promoter, enhancer, a sequence encoding a signal sequence, and/or a sequence encoding an affinity purification sequence. One of ordinary skill in the art is familiar with selecting appropriate sequence(s) or vector(s) and using them. The polypeptides and the nucleic acids that encode the polypeptides of the invention may additionally have or encode a selected signal sequence region and/or an affinity purification sequence region. As used herein, the term “signal sequence or signal peptide” is understood to mean a peptide segment which directs the secretion of the kringle or abrogen polypeptide or fusion polypeptides and thereafter is cleaved following translation in the host cell. The signal sequence or signal peptide can thus initiate transport of a protein across the membrane of the endoplasmic reticulum. Signal sequences have been well characterized in the art and are known typically to contain 16 to 30 amino acid residues, and may contain greater or fewer amino acid residues. A typical signal peptide consists of three regions: a basic N-terminal region, a central hydrophobic region, and a more polar C-terminal region. The central hydrophobic region contains 4 to 12 hydrophobic residues that anchor the signal peptide across the membrane lipid bilayer during transport of the nascent polypeptide. Following initiation, the signal peptide is usually cleaved within the lumen of the endoplasmic reticulum by cellular enzymes known as signal peptidases (von Heijne (1986) Nucleic Acids Res., 14: 4683). Numerous examples exist including the well known poly-His tag sequence, the immunoglobulin signal sequence, and the human interleukin 2 (IL2) signal sequence.

The polypeptide and the sequence encoding the polypeptide used in a specific vector encoding the kringle or abrogen sequence may also be linked to stabilizing elements or polypeptides or the sequences that encode them, such as those from human serum albumin or the immunoglobulin Fc portion of an IgG molecule.

The abrogen polypeptides according to the present invention may be advantageously linked to a fusion partner as known in the art, such as human serum albumin (HSA). Such fusions polypeptides comprise the abrogen polypeptides fused at either or both of the C- or N-terminal with HSA. The amino acid sequence of HSA is well known in the art and is inter alia disclosed by Meloun et al. (Complete Amino Acid Sequence of HSA, FEBS Letter: 58:1. 136-137, 1975) and Behrens et al. (Structure of HSA, Fed. Proc. 34,591, 1975), and more recently by genetic analysis (Lawn et al., Nucleic Acids Research, 1981, 9, 6102-6114). Shorter forms or variants as described in EP 322 094 of HSA may also be used to produce the kringle or abrogen fusion protein. Construction of such fusion proteins is well known in the art and is disclosed inter alia, in U.S. Pat. No. 5,876,969. Fusion proteins so obtained possess a particularly advantageous distribution in the body, while modifying their pharmacokinetic properties, and favoring the development of their biological activity. Many possible fusion partners or combinations of fusion partners can be selected for use and used at either or both ends of a kringle polypeptide or abrogen. As used throughout this document, a “kringle” polypeptide or “abrogen” polypeptide can also refer to a polypeptide that comprises the novel recombinant kringle domain and/or abrogen activity described here, so that all fusion proteins and combinations with one or more different or the same fusion partners are specifically included in these terms.

The kringle or abrogen fusion polypeptide according to a preferred aspect of the present invention may optionally comprise an N-terminal signal peptide such as the IL2 signal peptide providing for secretion into the surrounding medium, followed or preceded or both by a HSA or a portion thereof, or a variant thereof, and the sequence of the kringle of abrogen polypeptide. The polypeptides may be coupled either directly or via an artificial peptide or linker to albumin at the N-terminal end or the C-terminal end or at both ends.

The chimeric molecules may be produced by eucaryotic or prokaryotic cellular hosts that contain a nucleotide sequence encoding the fusion protein, and then harvesting the polypeptide produced. Animal cells, yeast, fungi may be used as eucaryotic hosts. In particular, yeast of the genus of Saccharomyces, Kluveromyces, Pichia, Schwanniomyces, or Hansenula may be used. Animal cells such as for example, COS, CHO, 293 cell lines, and C127 cells, and the like may be used. Fungi such as Aspergillus sp., or Trichodenna ssp may be used. Bacteria, such as Esherichia coli, or bacteria belonging to the genera of Corynebacterium, Bacillus, or Streptomyces may be used as prokaryotic cells, and, archaebacteria may be selected as well.

Alternatively, the fusion polypeptide can be one formed by the fusion of one or more immunoglobulin Fc region as described in WO 00/01133. Immunoglobulin Fc region is understood to mean the carboxylterminal portion of an immunoglobulin chain constant region, preferably an immunoglobulin heavy chain constant region, or a portion thereof. For example, an immunoglobulin Fc region may comprise: 1) an immunoglobulin constant heavy 1 (CH1) domain, an immunoglobulin constant heavy 2 (CH2) domain, and an immunoglobulin constant heavy (CH3) domain; 2) a CH1 domain and a CH2 domain; 3) a CH1 domain and a CH3 domain; 4) a CH2 domain and a CH3 domain; or 5) a combination of two or more domains and an immunoglobulin hinge region. In a preferred embodiment the Fe region used in the DNA construct includes at least an immunoglobulin hinge region, CH2 and CH3 domains, and depending upon the type of immunoglobulin used to generate the Fe region, optionally a CH4 domain. More preferably, the immunoglobulin Fe region comprises a hinge region, and CH2 and CH3 domains. Immunoglobulin from which the heavy chain constant region is preferably derived is IgG of subclasses 1, 2, 3, or 4, and most preferably of subclass 2, most preferably the murin or human immunoglobulin Fe region from IgG2a. Other classes of immunoglobulin, IgA, IgD, IgE and IgM, may be used. The choice of appropriate immunoglobulin heavy chain constant regions is discussed in detail in U.S. Pat. Nos. 5,541,087, and 5,726,044. The choice of particular immunoglobulin heavy chain constant region sequences from certain immunoglobulin classes and subclasses to achieve a particular result is considered to be within the level of skill in the art. The Fe region used in the fusion protein is preferably from a mammalian species, for example of murine origin, and preferably from a human or humanized Fe region.

The polypeptides or fusion proteins of the invention preferably are generated by conventional recombinant DNA methodologies. The polypeptides or fusion proteins preferably are produced by expression in a host cell of a DNA molecule encoding a signal sequence, an immunoglobulin Fc region and a kringle or abrogen polypeptide. The constructs may encode in a 5′ to 3′ direction, the signal sequence, the immunoglobulin Fe region and the abrogen protein. Alternatively, the constructs may encode in a 5′ to 3′ direction, the signal sequence, the kringle or abrogen polypeptide and the immunoglobulin Fe region. Also, the contructs may encode a signal sequence, an immunoglubulin Fe region, the kringle or abrogen polypeptide, and another immunoglobulin Fe region. As noted above, other fusion partner proteins or frgaments thereof, such as HSA, can be selected ans substituted for either or both of the HSA and immunoglobulin Fe region examples given above. One of skill in the art is familiar with numerous fusion partner examples. In addition, the polypeptide may be coupled either directly or via a linker to the one or more immunoglobulin Fe regions or fusion partners. The fusion of the polypeptide with immunoglobulin Fe region is produced by introducing into mammalian cell such constructs, and culturing the mammalian cells to produce the fusion proteins. The resulting fusion proteins can be harvested, refolded if necessary, and purified using conventional purification techniques well known and used in the art. The resulting fusion polypeptides exhibit longer serum half-lives, presumably due to their larger molecular sizes. Combinations of HSA and immunoglobulin Fe region or any other combination of other fusion partners or stabilizing proteins useful as a fusion proten, can be selected in embodiments where two or more proteins or regions are linked to the kringle or abrogen polypeptide.

In a preferred embodiment, kringle or abrogen polypeptides and either one or more or both of the HSA or the immunoglobulin Fe region may be linked by a polypeptide linker. As used herein the term “polypeptide linker” is understood to mean a peptide sequence that can link two proteins together or a protein and an Fe region. The polypeptide linker preferably comprises a plurality of amino acids such as glycine and/or serine. Preferably, the polypeptide linker comprises a series of glycine and serine peptides about 10-15 residues in length. See, for example, U.S. Pat. No. 5,258,698, the disclosure of which is incorporated herein by reference. More preferably, the linker sequence is as set forth in SEQ ID NO: 32 or 36, or comprises an Asp-Ala or an Arg-Leu sequence. It is contemplated however, that the optimal linker sequence length and amino acid composition may be determined by routine experimentation.

The invention also relates to recombinant vectors containing the isolated nucleic acid sequence of any one of sequences SEQ ID NO: 15-28, and host cells comprising the nucleic acid sequence of any one of sequences SEQ ID NO: 15-28. Similarly, the invention includes methods for making such vectors and host cells and for using them for the production of one or more kringle polypeptides.

A cell can be transduced with, transfected with, or have introduced into it a vector or nucleic acid that comprises the kringle polypeptide or abrogen activity encoding nucleic acid. Progeny of any of the cells mentioned, for example cells that result from cultured cell splitting or maintenance procedures, are also included in the invention. The cell can be a cultured primary cell, an established cell line cell, a transformed cell, a tumor cell, an endothelial cell, or a variety of other mammalian cells.

Additionally, various promoter/enhancer and RNA transcript stabilizing elements may be included in a vector of the invention.

Preferably, inhibiting tube formation in endothelial cell cultures induced by bFGF and VEGF, reducing cell proliferation induced by bFGF and VEGF, and/or inhibiting metastasis of mammalian tumors is measured in culture with established endothelial cell lines or tumor cell lines. However, other types of measurements, including measurements in vivo, can also be used. In this and other aspects of the invention involving cells, a preferred embodiment employs or involves human umbilical vein endothelial cells or mammary or lung tumor cells.

As shown here, the kringle or Abrogen polypeptides having the amino acid sequence of SEQ ID Nos: 1-14 or the nucleic acids encoding them, such as those having the nucleic acid sequences as set forth in SEQ ID NOs: 15-28, can be identified and used to inhibit or reduce tumor metastasis, inhibit or reduce endothelial cell proliferation induced by both bFGF and VEGF either in separate assays or together in one assay, and/or inhibit or reduce endothelial cell tubule formation. Additional examples have been mentioned and/or are described below in their structure and/or method of making and identifying. Functionally, a kringle polypeptide of the invention can be distinguished by at least the ability to inhibit tumor metastasis. The kringle or Abrogen polypeptides can be either secreted or expressed inside a cell. In preferred examples, the kringle or Abrogen polypeptide is expressed in substantially soluble form.

In making and using aspects and embodiments of this invention, one skilled in the art may employ conventional molecular biology, cell biology, virology, microbiology, and recombinant DNA techniques. Exemplary techniques are explained fully in the literature. For example, one may rely on the following general texts to make and use the invention: Sambrook et al., Molecular Cloning: A Laboratory Manual, Second Edition (1989) Cold Spring Harbor Laboratory Press, Cold Spring Harbor, N.Y. and Sambrook et al., Third Edition (2001); DNA Cloning: A Practical Approach, Volumes I and II (D. N. Glover ed. 1985); Oligonucleotide Synthesis (M. J. Gaited. 1984); Nucleic Acid Hybridization (B. D. Hames & S. J. Higgins eds. (1985)); Transcription And Translation, Hames & Higgins, eds. (1984); Animal Cell Culture (R I. Freshney, ed. (1986)); Immobilized Cells And Enzymes (IRL Press, (1986)); Gennaro et al. (eds.) Remington's Pharmaceutical Sciences, 18th edition; B. Perbal, A Practical Guide To Molecular Cloning (1984); F. M. Ausubel et al. (eds.), Current Protocols in Molecular Biology, John Wiley & Sons, Inc. (2001), Coligan et al. (eds.), Current Protocols in Immunology, John Wiley & Sons, Inc. (2001); W. Paul et al. (eds.) Fundamental Immunology, Raven Press; E. J. Murray et al. (ed.) Methods in Molecular Biology: Gene Transfer and Expression Protocols, The Humana Press Inc. (1991); J. E. Celis et al., Cell Biology: A Laboratory Handbook, Academic Press (1994); J. E. Coligan et al. (Eds.) Current Protocols in Protein Science, John Wiley & Sons (2001); and J. S. Bonifacino et al. (Eds.) Current Protocols in Cell Biology, John Wiley & Sons, Inc. (2001). Additional information sources are listed below or are referred to by citation number corresponding to the references at the end of the specification.

The present invention also encompasses Abrogen polypeptide derivatives, which include those having one or more conservative amino acid substitutions. For example, one or more amino acid residues within a sequence can be substituted by another amino acid of a similar polarity, which acts as a functional equivalent when the substitution results in no significant change in activity in at least one selected biological activity or function.

Substitutions for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

“Isolated,” when referring to a nucleic acid or polypeptide, means that the indicated molecule is present in the substantial absence of at least one other molecule with which it naturally occurs or necessarily occurs because of its method of preparation. Thus, for example, an “isolated Abrogen polypeptide” refers to a molecule substantially free of a macromolecule existing in a cell used to produce the abrogen polypeptide. However, the preparation or sample containing the molecule may include other components of different types. In addition, “isolated from” a particular molecule may also mean that a particular molecule is substantially absent from a preparation or sample. Varying degrees of isolation can be prepared from methods known in the art. Similarly, a “purified” form of a molecule is at least partially separated from a final reaction mixture that produces it, or one or more components of a mixture containing it have been substantially or to a measurable extent removed. A purified form can also be a form suitable for pharmaceutical research use, such as a form substantially free of antigenic or inflammatory components. A purified form can also be the result of an affinity purification process or any other purification step or process.

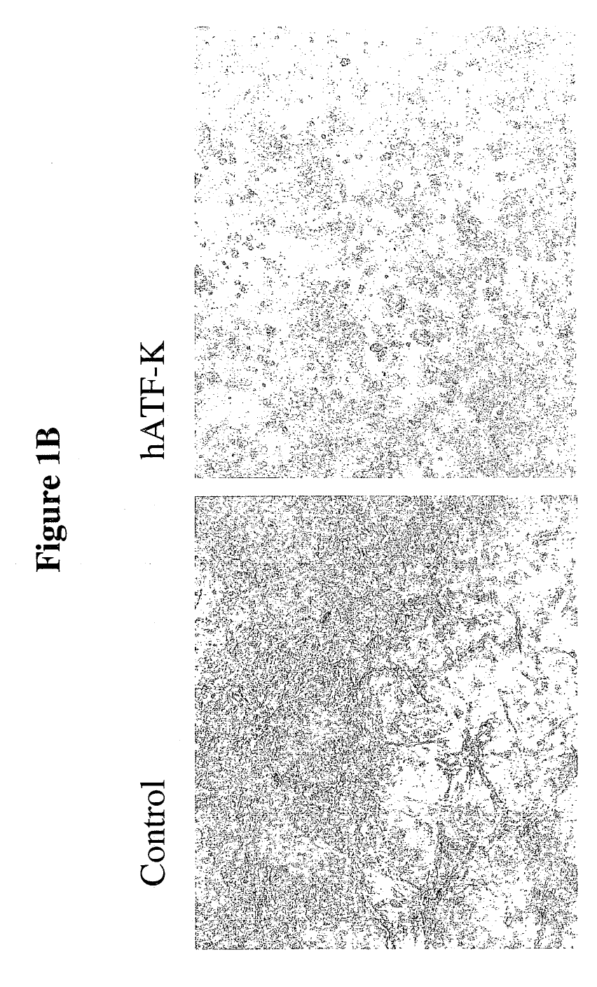

The “derivatives” noted here can be produced using homologue sequences, modifications of an existing sequence, or a combination of the two. The term “homologue” is used herein to refer to similar or homologous sequences, whether or not any particular position or residue is identical to or different from the molecule similarity or homology is measured against. A nucleic acid or amino acid sequence alignment may include spaces. Preferably, alignment is made using the consensus residues as listed in FIG. 2, or the 6 Cys residues of the kringle domain. One way of defining a homologue is through “percent identity” between two nucleic acids or two polypeptide molecules. This refers to the percent defined by a comparison using a basic blastn or blastp or blastx algorithm at the default setting, unless otherwise indicated (see, for example, NCBI BLAST home page: http://www.ncbi.nlm.nih.gov/BLAST/). Aligning a Cys residue in a kringle polypeptide, for example, can be performed by comparing sequences where the first amino acid residue or codon is for a particular Cys, or where the particular Cys residue is set at the same position as that of the abrogen Cys residue. For example, the blastp algorithm was used to generate homolog sequences, as in those of FIG. 2, by selecting the Blosum62 matrix, gap costs set at Existence: 11 and Extension: 1 (the default settings when performed). Typically, the default setting is used unless otherwise indicated. “Homology” can be determined by a direct comparison of the sequence information between two polypeptide molecules by aligning the sequences and using readily available computer programs. Alternatively, homology can be determined by hybridization of polynucleotides under conditions allowing for the formation of stable duplexes between homologous regions and determining or identifying the presence of double-stranded nucleic acid.

A “functional homologue” or a “functional equivalent” of a given polypeptide or sequence includes molecules derived from the native polypeptide sequence, as well as recombinantly produced or chemically synthesized polypeptides, which function in a manner similar to the reference molecule or achieve a similar desired result. Thus, a “functional homologue” or a “functional equivalent” of a given kringle nucleotide region includes similar regions derived from a different species, nucleotide regions derived from an isoform, or from a different cellular source, or resulting from an alternative splicing event, as well as recombinantly produced or chemically synthesized nucleic acids that function in a manner similar to the reference nucleic acid region in achieving a desired result, such as a result in a particular assay or cell characteristic.

A “recombinant” molecule is one that has undergone at least one molecular biological manipulation, as known in the art. Typically, this manipulation occurs in vitro but it can also occur within a cell, as with homologous recombination. A recombinant polypeptide is one that is produced from a recombinant DNA or nucleic acid. A “coding sequence” or “sequence that encodes” is a sequence capable of being transcribed and translated into a polypeptide in a cell in vitro or in vivo when placed under the control of appropriate regulatory sequences. The boundaries of the coding sequence are determined by a start codon at the 5 ′ (amino) terminus and a translation stop codon at the 3′(carboxyl) terminus.

A “nucleic acid” is a polymeric compound comprised of covalently linked nucleotides, from whatever source. Nucleic acid includes polyribonucleic acid (RNA) and polydeoxyribonucleic acid (DNA), both of which may be single-stranded or double stranded DNA includes cDNA, genomic DNA, synthetic DNA, and semisynthetic DNA. The term “nucleic acid” also captures sequences that include any of the known base analogues of DNA and RNA.

As used herein, a “vector” means any nucleic acid or nucleic acid-bearing particle, cell, or organism capable of being used to transfer a nucleic acid into a host cell and/or used to cause the expression of a polypeptide in a host cell. The term “vector” includes both viral and nonviral products and means for introducing the nucleic acid into a cell. A “vector” can be used in vitro, ex vivo, or in vivo. Non-viral vectors include plasmids, cosmids, and can comprise liposomes, electrically charged lipids (cytofectins), DNA protein complexes, and biopolymers, for example. Viral vectors include retroviruses, lentiviruses, adeno-associated virus, pox viruses, baculovirus, reoviruses, vaccinia viruses, herpes simplex viruses, Epstein-Barr viruses, and adenovirus vectors, for example. Vectors can also comprise the entire genome sequence or recombinant genome sequence of a virus. A vector can also comprise a portion of the genome that comprises the functional sequences for production of a virus capable of infecting, entering, or being introduced to a cell to deliver nucleic acid therein.

A cell has been “transfected” by a vector or exogenous or heterologous nucleic acid when the vector or nucleic acid has been introduced inside the cell. A cell has been “transformed” or “transduced” by a vector or exogenous or heterologous nucleic acid when the vector or nucleic acid effects a phenotypic change or detectable modification in the cell, such as expression of a polypeptide.

Viral vectors commonly used for in vivo or ex vivo targeting and therapy procedures are DNA-based vectors and retroviral vectors. Methods for constructing and using viral vectors are known in the art (see, e.g., Miller and Rosman, BioTechniques 7:980-990 (1992)). Preferably, the viral vectors are replication defective or conditionally replication defective, that is, they are unable to replicate autonomously in the target cell or unable to replicate autonomously under certain conditions. In general, the genome of the replication defective viral vectors which are used within the scope of the present invention lack at least one region which is necessary for the replication of the virus in the infected cell. These regions can either be eliminated (in whole or in part), be rendered non-functional by any technique known to a person skilled in the art. These techniques include the total removal, substitution (by other sequences, in particular by the inserted nucleic acid), partial deletion or addition of one or more bases to an essential (for replication) region. Such techniques may be performed in vitro (on the isolated DNA) or in situ, using the techniques of genetic manipulation or by treatment with mutagenic agents. Preferably, the replication defective virus retains the sequences of its genome necessary for encapsulating the viral particles.

DNA viral vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt et al., Molec. Cell. Neurosci. 2:320-330 (1991)), defective herpes virus vector lacking a glyco-protein L gene, or other defective herpes virus vectors (PCT Publication WO 94/21807 and WO 92/05263); an attenuated adenovirus vector, such as the vector described by Stratford-Perricaudet et al. (J. Clin. Invest. 90:626-630 (1992); see also La Salle et al., Science 259:988-990 (1993)); a defective adeno-associated virus vector (Samulski et al., J. Virol. 61:3096-3101 (1987); Samulski et al., J. Virol. 63:3822-3828 (1989); Lebkowski et al., Mol. Cell. Biol. 8:3988-3996 (1988)); and a conditional replicative recombinant vectors (see, for example, U.S. Pat. Nos. 6,111,243, 5,972,706, and published PCT documents WO 00136650, WO 0024408).

Recombinant adenoviruses display many advantages for use as transgene expression systems, including a tropism for both dividing and non-dividing cells, minimal pathogenic potential, ability to replicate to high titer for preparation of vector stocks, and the potential to carry large inserts (see e.g., Berkner, K. L., Curr. Top. Micro. Immunol., 158:39-66 (1992); Jolly D., Cancer Gene Therapy, 1:51-64 (1994)).

It is also possible to introduce the vector in vivo as a naked DNA plasmid. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter or eletrotransfer device (see, e.g., Wu et al., J. Biol. Chem. 267:963-967 (1992); Wu and Wu, J. Biol. Chem. 263:14621-14624 (1988); Hartmut et al., Canadian Patent Application No. 2,012,311, filed Mar. 15, 1990; Williams et al., Proc. Natl. Acad. Sci. USA 88:2726-2730 (1991)). Receptor-mediated DNA delivery approaches can also be used (Curiel et al., Hum. Gene Ther. 3:147-154 (1992); Wu and Wu, J. Biol. Chem. 262:4429-4432 (1987)). Naked plasmids or cosmids can be used in a number of gene transfer protocols and these plasmids and cosmids can be used in embodiments of this invention (see, in general, Miyake et al., PNAS 93:1320-1324 (1996); U.S. Pat. No. 6,143,530; U.S. Pat. No. 6,153,597; Ding et al., Cancer Res., 61:526-31 (2001); and Crouzet et al., PNAS 94:1414-1419 (1997). Among the preferred plamid vectors are those described in WO9710343 and WO9626270. Plasmids can also be combined with lipid compositions, pharmaceutically acceptable vehicles, and used with electrotransfer technology, as known in the art (see, for example, U.S. Pat. Nos. 6,156,338 and 6,143,729, and WO9901157 and the related devices in WO9901175).

In a second aspect, the invention provides a composition containing at least one Abrogen polypeptide having a sequence as in any one of SEQ ID NO: 1-14, a fusion construct or a derivative thereof as described herein above, for administration to a cell in vitro and/or in vivo or to a multicellular organism. In preferred embodiments of this aspect, the composition comprises at least one Abrogen polypeptide for expression thereof in a host organism for treatment of angiogenesis related disease.

The invention also provides for a pharmaceutical composition comprising an appropriate amount of at least one Abrogen polypeptide, a fusion construct or a derivative thereof as described herein and a pharmaceutically acceptable carrier. The use of an effective amount of at least one Abrogen polypeptide, a fusion construct or a derivative thereof for the preparation of a composition or a drug for treatment or prevention or a angiogenesis related disease is also provided.

The pharmaceutical composition or drug according to the invention may be employed for instance to treat angiogenesis related diseases, such as diabetic retinopathy, macular degeneration, obesity, rheumatoid arthritis, and psoriasis. Further use of the Abrogens polypeptides includes the prevention of tumors and/or reduction and/or prevention of growth in tumors. Methods of treating individuals are also provided.

The use of the kringle domain of the proteins selected from the group consisting of factor XII, the hepatocyte growth factor activator, the hyaluronan binding protein, the neurotrypsin, the retinoic acid-related receptors 1 and 2 (ROR-1 and ROR-2), the kremen protein, the t-PALP, the ApoArgC, the macrophage stimulating proteins (MSP), and thrombin allows greater specificity in the antiangiogenic mode of action. Data from in vitro studies show that the Abrogen polypeptides according to the present invention possess a new activity that inhibits both bFGF and VEGF induced tube formation and/or cell proliferation in specific endothelial cell assays. This assay also distinguishes the species-specific activity of other anti-angiogenic polypeptides. In another contrast over previous polypeptides, anti-angiogenic factors such as endostatin or angiostatin only inhibit bFGF-induced activity in this assay (Chen et al., Hum Gen Ther 11: 1983-96 (2000)).

As noted above, a number of compositions comprising an appropriate or effective amount of one or more abrogen polypeptides can be prepared. Combinations of two or more isolated or purified Abrogen polypeptides can be prepared.

In addition, combinations of one or more abrogen polypeptides with one or more conventional therapies, such as radiotherapy, chemotherapy, or surgery can be used. Further combinations of one or more abrogen polypeptides with another biologically active compound, such as a therapeutic compound, can be prepared. Any available compound can be used in the combination, including approved therapeutic compounds.

The compositions of the present invention may be provided to an animal by any suitable means, directly (e.g., locally, as by injection, implantation or topical administration to a tissue locus) or systemically (e.g., parenterally or orally). Where the composition is to be provided parenterally, such as by intravenous, subcutaneous, ophthalmic (including intravitreal or intracameral), intraperitoneal, intramuscular, buccal, rectal, vaginal, intraorbital, intracerebral, intracranial, intraspinal, intraventricular, intrathecal, intracistemal, intracapsular, intranasal or by aerosol administration, the composition preferably comprises part of an aqueous or physiologically compatible fluid suspension or solution. Thus, the carrier or vehicle is physiologically acceptable so that in addition to delivery of the desired composition to the patient, it does not otherwise adversely affect the patient's electrolyte and/or volume balance. The fluid medium for the agent thus can comprise normal physiologic saline (e.g., 9.85% aqueous NaCl, 0.15 M, pH 7-7.4). In one embodiment, the composition is a pharmaceutically acceptable composition. One skilled in the art is familiar with selecting and testing pharmaceutically acceptable compositions for use with recombinant polypeptides and nucleic acids.

The formulations may conveniently be presented in unit dosage form and may be prepared by conventional pharmaceutical techniques. Such techniques include the step of bringing into association the active ingredient and the pharmaceutical carrier(s) or excipient(s).

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

Moreover, an abrogen polypeptide may be used a therapeutic. The polypeptide and the method for expressing it in a cell can be, therefore, used in methods to treat or prevent a variety of angiogenesis related diseases or conditions, including, but not limited to hemangioma, solid tumors, blood borne tumors, leukemia, metastasis, telangiectasia, psoriasis, scleroderma, pyogenic granuloma, myocardial angiogenesis, Crohn's disease, plaque neovascularization, coronary collaterals, cerebral collaterals, arteriovenous malformations, ischemic limb angiogenesis, corneal diseases, rubeosis, neovascular glaucoma, diabetic retinopathy, retrolental fibroplasia, arthritis, rheumatoid arthritis, diabetic neovascularization, diabetic retinopathy, macular degeneration, wound healing, obesity, peptic ulcer, Helicobacter related diseases, fractures, keloids, vasculogenesis, hematopoiesis, ovulation, menstruation, placentation, psoriasis, and cat scratch fever.

In general, the use can also be for abrogating tumor vasculature growth or angiogenesis associated with a tumor. One skilled in the art is familiar with polypeptide expression and purification systems as well as methods for administering polypeptides and vectors in appropriate pharmaceutical compositions.

The kringle or abrogen polypeptides or fusion proteins thereof can also be used in combination with other therapeutic agents and a combination with multiple, different kringle or abrogen or fusion polypeptides can also be selected. Any existing or available therapeutic treatments can be combined with the polypeptides, combinations, or methods described here. Numerous examples exist and the compounds and the treatment methods can be selected from those available, such as those in the Physician's Desk Reference, Remington's Pharmaceutical Sciences, or Remington's Science and Practice of Pharmacy. A combination with an erythropoietin is specifically noted. Combinations with treatments or compounds that implicate angiogenesis or anti-angiogenesis mechanisms are preferred, but other tumor suppressing treatments and anti-cancer treatments or treatments used in cancer patients can also be selected.

The administration of abrogen polypeptide with a parallel administration of the second biologically active compound can comprise treatment regimens where one is administered first, followed by the other, where both are administered at the same time, where one is administered for a period of time and the other for another period of time, or combinations of any of these regimens. The mode of administration would be intramuscular, intratumoral, intraperitoneal, intracranial or intraveneous.

The combination according to the present invention can be administered, especially for tumor therapy, in combination with chemotherapy, radiotherapy, immunotherapy, surgical intervention, or a combination of these. Long-term therapy is equally possible as is adjuvant therapy in the context of other treatment strategies, as described above.

Therapeutic agents for possible combination are one or more cytostatic or cytotoxic compounds, for example a chemotherapeutic agent or one or several selected from the group comprising an inhibitor of polyamine biosynthesis, an inhibitor of protein kinase, especially of serine/threonine protein kinase, such as protein kinase C, or of tyrosine protein kinase, such as epidermal growth factor receptor tyrosine kinase, a cytokine, a negative growth regulator, such as TGF-β or IFN-β, an aromatase inhibitor, a classical cytostatic, and an inhibitor of the interaction of an SH2 domain with a phosphorylated protein.

The pharmaceutical compositions according to the present invention can be used in a method for the prophylactic or especially therapeutic treatment of angiogenesis related disease, especially those mentioned hereinabove, as well as tumor diseases.

Preference is given to the use of solutions of the active ingredient, and also suspensions or dispersions, especially isotonic aqueous solutions, dispersions or suspensions which, for example in the case of lyophilised compositions comprising the active ingredient alone or together with a carrier, for example mannitol, can be made up before use. The pharmaceutical compositions may be sterilized and/or may comprise excipients, for example preservatives, stabilizers, wetting agents and/or emulsifiers, solubilizers, salts for regulating osmotic pressure and/or buffers and are prepared in a manner known per se, for example by means of conventional dissolving and lyophilizing processes. The said solutions or suspensions may comprise viscosity-increasing agents, typically sodium carboxymethylcellulose, carboxymethylcellulose, dextran, polyvinylpyrrolidone, or gelatins, or also solubilizers.

Suspensions in oil comprise as the oil component the vegetable, synthetic, or semi-synthetic oils customary for injection purposes. In respect of such, special mention may be made of liquid fatty acid esters that contain as the acid component a long-chained fatty acid having from 8 to 22, especially from 12 to 22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brassidic acid or linoleic acid, if desired with the addition of antioxidants, for example vitamin E, p-carotene or 3,5-ditert-butyl-4-hydroxytoluene. The alcohol component of these fatty acid esters has a maximum of 6 carbon atoms and is a monovalent or polyvalent, for example a mono-, di-or trivalent, alcohol, for example methanol, ethanol, propanol, butanol or pentanol or the isomers thereof, but especially glycol and glycerol. As fatty acid esters, therefore, the following are mentioned: ethyl oleate, isopropyl myristate, isopropyl palmitate, “Labrafil M 2375” (polyoxyethylene glycerol trioleate from Gattefoss Paris), “Labrafil M 1944 CS” (unsaturated polyglycolized glycerides prepared by alcoholysis of apricot kernel oil and consisting of glycerides and polyethylene glycol ester; Gattefosse, France), “Labrasol” (saturated polyglycolized glycerides prepared by alcoholysis of TCM and consisting of glycerides and polyethylene glycol ester; Gattefosse, France), and/or “Miglyol 812” (triglyceride of saturated fatty acids of chain length C9 to C12 from Huis AG, Germany), but especially vegetable oils such as cottonseed oil, almond oil, olive oil, castor oil, sesame oil, soybean oil and more especially groundnut oil.

The manufacture of injectable preparations is usually carried out under sterile conditions, as is the filling, for example, into ampoules or vials, and the sealing of the containers.

Pharmaceutical compositions for oral administration can be obtained, for example, by combining the active ingredient with one or more solid carriers, if desired granulating a resulting mixture, and processing the mixture or granules, if desired or necessary, by the inclusion of additional excipients, to form tablets or tablet cores.

Suitable carriers are especially fillers, such as sugars, for example lactose, saccharose, mannitol or sorbitol, cellulose preparations, and/or calcium phosphates, for example tricalcium phosphate or calcium hydrogen phosphate, and also binders, such as starches, for example corn, wheat, rice or potato starch, methylcellulose, hydroxypropyl methylcellulose, sodium carboxymethylcellulose, and/or polyvinylpyrrolidone, and/or, if desired, disintegrators, such as the above-mentioned starches, also carboxymethyl starch, crosslinked polyvinylpyrrolidone, alginic acid or a salt thereof, such as sodium alginate. Additional excipients are especially flow conditioners and lubricants, for example silicic acid, talc, stearic acid or salts thereof, such as magnesium or calcium stearate, and/or polyethylene glycol, or derivatives thereof.

Tablet cores can be provided with suitable, optionally enteric, coatings through the use of, inter alia, concentrated sugar solutions which may comprise gum arabic, talc, polyvinylpyr-rolidone, polyethylene glycol and/or titanium dioxide, or coating solutions in suitable organic solvents or solvent mixtures, or, for the preparation of enteric coatings, solutions of suitable cellulose preparations, such as acetylcellulose phthalate or hydroxypropylmethylcellulose phthalate. Dyes or pigments may be added to the tablets or tablet coatings, for example for identification purposes or to indicate different doses of active ingredient.

Pharmaceutical compositions for oral administration also include hard capsules consisting of gelatin, and also soft, sealed capsules consisting of gelatin and a plasticizer, such as glycerol or sorbitol. The hard capsules may contain the active ingredient in the form of granules, for example in admixture with fillers, such as cornstarch, binders, and/or glidants, such as talc or magnesium stearate, and optionally stabilizers. In soft capsules, the active ingredient is preferably dissolved or suspended in suitable liquid excipients, such as fatty oils, paraffin oil or liquid polyethylene glycols or fatty acid esters of ethylene or propylene glycol, to which stabilizers and detergents, for example of the polyoxyethylene sorbitan fatty acid ester type, may also be added.

For parenteral administration, aqueous solutions of an active ingredient in water-soluble form, for example of a water-soluble salt, or aqueous injection suspensions that contain viscosity-increasing substances, for example sodium carboxymethylcellulose, sorbitol and/or dextran, and, if desired, stabilizers, are especially suitable. The active ingredient, optionally together with excipients, can also be in the form of a lyophilizate and can be made into a solution before parenteral administration by the addition of suitable solvents.

Solutions such as are used, for example, for parenteral administration can also be employed as infusion solutions.

Preferred preservatives are, for example, antioxidants, such as ascorbic acid, or microbicides, such as sorbic acid or benzoic acid.

The invention relates likewise to a process or a method for the treatment of one of the pathological conditions mentioned hereinabove, especially angiogenesis related diseases, or neoplastic disease.

The combination can be administered as such or especially in the form of pharmaceutical compositions, prophylactically or therapeutically, preferably in an amount effective against the said diseases, to a patient requiring such treatment. In the case of an individual having a bodyweight of about 70 kg the daily dose administered is from approximately 0.05 g to approximately 5 g, preferably from approximately 0.25 g to approximately 1.5 g, of a compound of the present invention.

In another aspect, the nucleic acids encoding an Abrogen polypeptide can be used in a gene transfer method. The examples show how recombinant plasmid and adenoviral vectors, for example, can be used to affect metastasis in a lung tumor model. Various gene transfer and gene therapy vectors can be used in conjunction with the nucleic acids of the invention to either analyze the activity of an abrogen polypeptide in vivo or treat, prevent, or ameliorate an angiogenesis-related disease or condition in an animal. Preferably, the animal is human or mouse. More particularly, nucleic acids of SEQ ID NO.: 15-28 can be cloned into a vector, preferably an adenoviral vector, an adeno-associated virus (AAV), a retroviral vector, a plasmid, or other suitable viral or non-viral vector. In one embodiment, the vector is administered to tumor bearing animal by direct intratumoral injection, intravenous injection, intramuscular injection, electrotransfer-mediated administration, or other suitable method. The efficacy of the polypeptide or fusion expressed from the vector can be assessed in the context of, for example, reduction of the primary tumor and/or abrogation of metastatic dissemination.

Accordingly, the invention comprises gene transfer methods and methods for expressing abrogen polypeptides in a cell of an animal. These methods may comprise inserting a selected kringle or abrogen encoding sequence, such as one encoding SEQ ID NO.: 1-14, into a mammalian expression vector or the expression cassette of an appropriate vector. The vector is administered to a cell of the animal by any number of methods available, including intratumoral injection, electrotransfer, infusion, subcutaneous injection, intramuscular injection, or intravenous administration. The effect of the expressed polypeptide can then be measured and compared to control. These methods can be used to treat any one of a number of angiogenesis related diseases or disorders, such as those listed above.

In a most preferred aspect, the invention comprises administration of at least one abrogen recombinant polypeptide in a cell of an animal. These methods may comprise administering the abrogen peptides as in SEQ ID NO: 1-14 by any well-known methods in the art, including for example, direct injections of the peptide at a specific site, i.e., by ophthalmic (including intravitreal or intraorbital), intraperitoneal, intramuscular, or intratumoral injections.

In a third aspect, the present invention encompasses a method for treating angiogenesis related diseases, wherein one or more Abrogen peptide, fusion or derivatives thereof, as described above, are administered. The present invention also encompasses method of treatment of diseases and processes that are mediated by angiogenesis. In a preferred embodiment, the Abrogen peptide is administered in combination with one or more therapeutic compounds, polypeptides or proteins. In a most preferred embodiment,

Thus, the present invention provides a method and composition for treating and/or preventing diseases and processes that are mediated by angiogenesis including, but not limited to, hemangioma, solid tumors, blood borne tumors, leukemia, metastasis, telangiectasia, psoriasis, scleroderma, pyogenic granuloma, myocardial angiogenesis, Crohn's disease, plaque neovascularization, coronary collaterals, cerebral collaterals, arteriovenous malformations, ischemic limb angiogenesis, corneal diseases, rubeosis, neovascular glaucoma, diabetic retinopathy, retrolental fibroplasia, arthritis, rheumatoid arthritis, diabetic neovascularization, diabetic retinopathy, macular degeneration, wound healing, peptic ulcer, Helicobacter related diseases, fractures, keloids, vasculogenesis, hematopoiesis, ovulation, menstruation, placentation, obesity, and cat scratch fever.

A method and a composition for treating or repressing and/or preventing the growth of a cancer are also provided.

The present invention also provide a method for treating ocular angiogenesis related diseases such as macular degeneration or diabetic retinopathy by direct ophthalmic injections of one of the Abrogens having the amino acid sequence SEQ ID NO: 1-14.

The present invention also provides a method for treating dermatological angiogenesis related disease, such as psiorasis, by subcutaneous administration of one of the Abrogens having the amino acid sequence SEQ ID NO: 1-14.

Still another preferred aspect of the present invention to provide a method for treating rheumatoid arthritis by administration of one of the Abrogens having the amino acid sequence SEQ ID NO: 1-14.

Another aspect of the present invention is to provide a method for targeted delivery of abrogen compositions to specific locations.

Yet another aspect of the invention is to provide compositions and methods useful for gene therapy for the modulation of angiogenic processes.

In these combination methods, the administration of abrogen polypeptide with a parallel administration of the second biologically active compound can comprise treatment regimens where one is administered first, followed by the other, where both are administered at the same time, where one is administered for a period of time and the other for another period of time, or combinations of any of these regimens. The mode of administration would be intramuscular, intratumoral, intraperitoneal, intracranial or intraveneous.

The combination according to the present invention can be administered especially for tumor therapy in combination with chemotherapy, radiotherapy, immunotherapy, surgical intervention, or a combination of these. Long-term therapy is equally possible as is adjuvant therapy in the context of other treatment strategies, as described above.

Therapeutic agents for possible combination are especially one or more cytostatic or cytotoxic compounds, for example a chemotherapeutic agent or several selected from the group comprising an inhibitor of polyamine biosynthesis, an inhibitor of protein kinase, especially of serine/threonine protein kinase, such as protein kinase C, or of tyrosine protein kinase, such as epidermal growth factor receptor tyrosine kinase, a cytokine, a negative growth regulator, such as TGF-β or IFN-β, an aromatase inhibitor, a classical cytostatic, and an inhibitor of the interaction of an SH2 domain with a phosphorylated protein.

In a fourth aspect, the present invention relates to a method of production and purification of Abrogen polypeptides in an active soluble form.

While the production of kringle-containing polypeptides has been previously discussed, the successful and efficient production of soluble forms of biologically active abrogen polypeptides from E. coli has not. An aspect of the invention, therefore, is the use of expression vectors and fusion protein constructs to efficiently produce soluble abrogen polypeptides from E. coli. A related aspect of the invention is the novel constructs and vectors that encode abrogen polypeptides and fusion proteins of abrogen polypeptides that can be used to express soluble abrogen polypeptides and fusions from E. coli. Advantageously, the methods, vectors, and constructs described and exemplified produce comparatively high levels of soluble fusion protein per gram of wet cell pellet. Furthermore, the ability to directly express measurable or high levels of soluble fusion protein from E. coli simplifies the purification and production of protein.

It is known that peptides and proteins may be produced via recombinant means in a variety of expression systems, such as various strains of bacterial, fungal, mammalian or insect cells. The production of small heterologous peptides recombinantly for effective research and therapeutic use encounters however several difficulties. They may be for example subject to intracellular degradation by proteases and peptidases present in the host cell. In particular, it has been previously reported that the various kringles of human plasminogen, i.e, kringles 2 (Eur. J. Biochem. 1994 219 p455), or kringle 3 (Eur. J. Biochem. 1994 219 p 455) or kringles 2 and 3 (Biochemistry 1996 35 p2357), or again kringle 4 (Biochemistry 2000 39 p74147419), are unable to adopt a stable soluble conformation when produced in E. coli. Therefore, the kringles are generally accumulated, and are found in the insoluble or “inclusion bodies” fraction, which render them almost useless for screening purposes in biological or biochemical assays. Furthermore, these inclusion bodies usually require further manipulations in order to solublize and refold the heterologous proteins. These additional steps are however technically difficult and expensive, in a high throughput project, that is for practical production of recombinant proteins for therapeutic, diagnostic or other research use.

Several different fusion protein partners with a desired heterologous peptide to protein are proposed in the art to enable the recombinant expression and or secretion of an heterologous protein. These fusions protein include inter alia LacZ, tipE fusion proteins, maltose binding protein fusions (MBP, Bedouelle et al., Eur. J. Biochem, 1988, 171(3): 541-9), the glutathione-S-transferase fusion protein (GST, Smith et al., Gene, 1988, 67(1): 31-40), the Z domain from the protein A (Z, Nilson et al., Protein Eng., 1987, 1:107-113), thioredoxin (TrxA, La Vallie et al., Biotechnology, 1993, 11: 187-193; Hoog et al., Biosci. Rep. 4:917, 1984), NusA (Davis et al., Biotechnol. Bioeng., 1999, 65: 382-388), and the Gb-i domain from the protein G (Gbl, Huth et al., Protein Sci., 1997, 6:2359-64), at the amino- or the carboxy-termini.

In this regard, Hammarstrom et al. (Protein Science 11:313 (2002) provides some discussion as to the effect of different fusions, namely GST, NusA, ZZ (double Z domain of protein A), Gb1, MBP, and TrxA, upon expression and solubilization of 32 potentially interesting human proteins having various characteristics in terms of size, cysteine content, and their solubility probability. While none appear outstanding, MBP seems to be somewhat better than the other fusion partners.

Kapust et al. (Protein Science 8:1668, 1999) also compared three soluble fusion partners MBP, TrxA, and GST to inhibit aggregation of six diverse proteins that normally accumulate in an insoluble form and reports that MBP is far more effective for solubilizing than the two other partners, in that the MBP fusion partners invariably proved to be more soluble than GST and TrxA, and thus rendered the protein capable of adopting a stably folded conformation.

However, neither Hammarstrom et al. nor Kapust et al. have specifically addressed the problem of solubility of peptides having a cysteine content of around at least 7%, and post-translational modifications such as the formation of disulfide bonds, although this refolding can be critical to produce or retain the activity of the protein. For instance, protein solubility is reported to be achieved in 74% of the tested proteins when fused to MPB or TRX but protein of MW around 10 kD with high cysteine content are found to be not soluble by Hammarstrom et al.

The Applicant has now discovered and shown that among the existing fusion partners the thioredoxin (TrxA) is in fact capable of providing a very advantageous effect in terms of solubility of fusion proteins having a cysteine content of around 7% and comprising 3 disulfide bonds, such as the abrogen polypeptides. This superior result was unexpected, as the previously existing guidelines have been found to be only partially predictable for producing stable, soluble and biologically active protein forms.

The invention thus comprises a method for producing a soluble abrogen polypeptide that comprises preparing a nucleic acid fusion construct comprising at least a TrxA-encoding sequence fused in frame to an abrogen polypeptide sequence. As in other aspects of the invention, the fusion partner encoding sequence can be located at the N-teminus, the C-terminus, or both ends of the abrogen encoding sequence, and different combinations of fusion partners can be selected for use. Preferably, the TrxA fusion partner is fused to the N-terminal of the abrogen. The amino acid sequence of the TrxA fusion partner are provided in SEQ ID NO: 22. The TrxA-abrogen fusion according to the invention may further comprise a linker peptide between the TrxA sequence and the abrogen sequence, which advantageously provides a selected cleavage site. Preferred cleavage site used is a thrombin cleavage site comprising the following amino acid sequence LVPRGS (SEQ ID NO: 23).

The present invention thus provides for an efficient method of increasing solubility of recombinant abrogen peptides. The abrogen produced by the method according to the present invention is obtained in an unexpected high soluble form. The fusion protein is cytoplasmic and can be easily recovered by lysing the bacteria, purified and cleaved using for example the thrombin cleavage site. The nucleic acid construct can be incorporated into a vector or otherwise manipulated into a cell in order to express the fusion abrogen polypeptide. To produce the TrxA-abrogen fusion protein of this invention, a host cell is either transformed with, or has integrated in its genome, a DNA molecule comprising the TrxA-abrogen fusion protein, preferably under the control of an expression control sequence capable of directing the expression of the fusion protein production. Any one of a number of available expression control sequences can be selected for use. In preferred embodiments, the expression control sequences can operate in bacterial cells, such as E. coli, in order to express soluble fusion protein in E. coli cultures or cells.

Host cells suitable for the present invention are preferably bacterial cells, such as the various strains of E. coli, which are well known host cells in the field of biotechnology. The E. coli strain BL21 lambda DE3, used in the Example, is preferably used, and most preferably the E. coli BL21 lambda DE3 trxB⁻ (Novagen), which has a mutation in the thioredoxine reductase (trxB gene) is used, thereby allowing for the formation of disulfide bond in E. coli cytoplasm.

The trxA-abrogen fusion protein may be purified by conventional procedures including selective precipitation solubilization and column chromatography methods. Preferably, a purification tag is included between the trxA and the abrogen sequence, eventually in upstream or downstream position of the cleavage proteolytic site for the thrombin (SEQ ID NO: 23). Purification tag sequences are well known in the art and include inter alia Arg-tag, calmodulin-binding peptide, cellulose binding domain, DsbA, c-myc-tag, FLAG-tag, HAT-tag, HIS-tag, and Strep-tag (Terpe K., Appl. Microbiol. Biotechnol, 2003, 60(5): 523-33). Preferably, the purification tags, such as a His tag sequence, and streptokinase tag which comprises a nine-amino acid peptide having intrinsic streptavidin binding activity, such as for examples the sequences AWRHPQFGG or WSHPQFEK (Lamla et al., Mol. Cell. Proteomics, 2002, 1(6): 46671) are used or incorporated into the fusion protein construct or vector. One or more cleavage sites to liberate abrogen polypeptide from the fusion protein can also be used in the fusion protein construct or vector.

### EXAMPLES

Surprisingly, our data now shows that kringle polypetides possessing an abrogen polypeptide can inhibit endothelial cell activation and/or proliferation mediated by several different proangiogenic proteins, such as basic fibroblast growth factor (bFGF) and vascular endothelial growth factor (VEGF), and in a species independent manner.

### Example 1

### Cloning and Manipulating Nucleic Acids

The primary nucleotide and polypeptide sequence listings corresponding to the human kringle angiogenic inhibitors or abrogens according to the present invention are shown below.

SEQ ID NO.:1: Amino acid sequence of the kringle domain of the factor XII
ASCYDGRGLSYRGLARTTLSGAPCQPWASEATYRNVTAEQARNWGLGGHAFCRNPDNDIRPWCFVLNRD
RLSWEYCDLAQCQT
SEQ ID NO.:2: Amino acid sequence of the kringle domain of the hepatocyte growth
FACTOR ACTIVATOR
ERCFLGNGTGYRGVASTSASGLSCLAWNSDLLYQELHVDSVGAAALLGLGPHAYCRNPDNDERPWCYVV
KDSALSWEYCRLEACES
SEQ ID NO.:3: Amino acid sequence of the kringle domain of the hyaluronan
binding protein
DDCYVGDGYSYRGKMNRTVNQHACLYWNSHLLLQENYNMFMEDAETHGIGEHNFCRNPDADEKPWCFIK
VTNDKVKWEYCDVSACSA
SEQ ID NO.:4: Amino acid sequence of the kringle domain of the neurotrypsin
WGCPAGEPWVSVTDFGAPCLRWAEVPPFLERSPPASWAQLRGQRHNFCRSPDGAGRPWCFYGDARGKVD
WGYCDCRH
SEQ ID NO.:5: Amino acid sequence of the kringle domain of the retinoic acid-related
orphan receptor ROR-1
HKCYNSTGVDYRGTVSVTKSGRQCQPWNSQYPHTHTFTALRFPELNGGHSYCRNPGNQKEAPWCFTLDE
NFKSDLCDIPACDS
SEQ ID NO.:6: Amino acid sequence of the kringle domain of the retinoic acid-related
orphan receptor ROR-2
QCYNGSGMDYRGTASTTKSGHQCQPWALQHPHSHHLSSTDFPELGGGHAYCRNPGGQMEGPWCFTQNKN
VRMELCDVPSCSP
SEQ ID NO.:7: Amino acid sequence of the kringle domain of the kremen protein
PECFTANGADYRGTQNWTALQGGKPCLFWNETFQHPYNTLKYPNGEGGLGEHNYCRNPDGDVSPWCYVA
EHEDGVYWKYCEIPACQM
SEQ ID NO.:8: Amino acid sequence of the kringle domain of the t-PALP
CGCFWDNGHLYREDQTSPAPGLRCLNWLDAQSGLASAPVSGAGNHSYCRNPDEDPRGPWCYVSGEAGVP
EKRPCEDLRCPE
SEQ ID NO.:9: Amino acid sequence of the kringle domain of the RGD receptor
KINASE
LACSHPFSKSATEHVQGHLGKKQVPPDLFQPYIEEICQNLRGDVFQKFIESDKFTRFCQWKNVELNIHL
TMNDFSVHRIIGRGGFGEVYGCRK
SEQ ID NO.:10: Amino acid sequence of the kringle domain of ApoArgC
QECYHSNGQSYRGTYFTTVTGRTCQAWSSMTPHQHSRTPEKYPNDGLISNYCRNPDGSAGPWCYTTDPN
VRWEYCNLTRCSD
SEQ ID NO.:11: Amino acid sequence of the kringle domain 1 of the macrophage
stimulating protein
RTCIMNNGVGYRGTMATTVGGLPCQAWSHKFPNDHKYTPTLRNGLEENFCRNPDGDPGGPWCYTTDPAV
RFQSCGTKSCRE
SEQ ID NO.:12: Amino acid sequence of the kringle domain 2 of the macrophage
stimulating protein
AACVWGNGEEYRGAVDRTESGRECQRWDLQHPHQHPFEPGKFLDQGLDDNYCRNPDGSERPWCYTTDPQ
IEREFCDLPRCGS
SEQ ID NO.:13: Amino acid sequence of the kringle domain 3 of the macrophage
stimulating protein
VSCFRGKGEGYRGTANTTTAGVPCQRWDAQIPHQHRFTPEKYAGKDLRENFCRNPDGSEAPWCFTLRPG
MRAAFCYQIRRCTD
SEQ ID NO.:14: Amino acid sequence of the kringle domain 4 of the macrophage
stimulating protein
QDCYHGAGEQYRGTVSKTRKGVQCQRWSAETPHKPQFTFTSEPHAQLEENFCRNPDGDSHGPWCYTMDP
RTPFDYCALRRCAD
SEQ ID NO.:15: Nucleotide sequence encoding the kringle domain of factor XII
GCAAGCTGCTATGATGGCCGCGGGCTCAGCTACCGCGGCCTGGCCAGGACCACGCTCTCGGGTGCGCCC
TGTCAGCCGTGGGCCTCGGAGGCCACCTACCGGAACGTGACTGCCGAGCAAGCGCGGAACTGGGGACTG
GGCGGCCACGCCTTCTGCCGGAACCCGGACAACGACATCCGCCCGTGGTGCTTCGTGCTGAACCGCGAC
CGGCTGAGCTGGGAGTACTGCGACCTGGCACAGTGCCAGACCTAG
SEQ ID NO.:16: Nucleotide sequence encoding the kringle domain of the hepatocyte
growth factor activator
GAGCGCTGCTTCTTGGGGAACGGCACTGGGTACCGTGGCGTGGCCAGCACCTCAGCCTCGGGCCTCAGC
TGCCTGGCCTGGAACTCCGATCTGCTCTACCAGGAGCTGCACGTGGACTCCGTGGGCGCCGCGGCCCTG
CTGGGCCTGGGCCCCCATGCCTACTGCCGGAATCCGGACAATGACGAGAGGCCCTGGTGCTACGTGGTG
AAGGACAGCGCGCTCTCCTGGGAGTACTGCCGCCTGGAGGCCTGCGAATCCTAG
SEQ ID NO.:17: Nucleotide sequence encoding the kringle domain of the hyaluronan
binding protein
GATGACTGCTATGTTGGCGATGGCTACTCTTACCGAGGGAAAATGAATAGGACAGTCAACCAGCATGCG
TGCCTTTACTGGAACTCCCACCTCCTCTTCCAGGAGAATTACAACATGTTTATGGAGGATGCTGAAACC
CATGCGATTGGGGAACACAATTTCTGCAGAAACCCAGATGCGGACGAAAAGCCCTGGTGCTTTATTAAA
GTTACCAATGACAAGGTGAAATGGGAATACTGTGATGTCTCAGCCTGCTCAGCCTAG
SEQ ID NO.:18: Nucleotide sequence encoding the kringle domain of the
neurotrypsin
TGGGGCTGCCCCGCCGGCGAGCCATGGGTCAGCGTGACGGACTTCGGCGCCCCGTGTCTGCGGTGGGCG
GAGGTGCCACCCTTCCTGGAGCGGTCGCCCCCAGCGAGCTGGGCTCAGCTGCGAGGACAGCGCCACAAC
TTTTGTCGGAGCCCCGACGGCGCGGGCAGACCCTGGTGTTTCTACGGAGACGCCCGTGGCAAGGTGGAC
TGGGGCTACTGCGACTGCAGACACTAG
SEQ ID NO.19: Nucleotide sequence encoding the kringle domain of the retinoic
acid-related receptor ROR-1
CACAAGTGTTATAACAGCACAGGTGTGGACTACCGGGGGACCGTCAGTGTGACCAAATCAGGGCGCCAG
TGCCAGCCATGGAACTCCCAGTATCCCCACACACACACTTTCACCGCCCTTCGTTTCCCAGAGCTGAAT
GCAGGCCATTCCTACTGCCGCAACCCAGGGAATCAAAAGGAAGCTCCCTGGTGCTTCACCTTGGATGAA
AACTTTAAGTCTGATCTGTGTGACATCCCAGCTTGCGATTCATAG
SEQ ID NO.20: Nucleotide sequence encoding the kringle domain of the retinoic
acid-related receptor ROR-2
CATCAGTGCTATAACGGCTCAGGCATGGATTACAGAGGAACGGCAAGCACCACCAAGTCAGGCCACCAG
TGCCAGCCGTGGGCCCTGCAGCACCCCCACAGCCACCACCTGTCCAGCACAGACTTCCCTGAGCTTGGA
GGGGGGCACGCCTACTGCCGGAACCCCGGAGGCCAGATGGAGGGCCCCTGGTGCTTTACGCAGAATAAA
AACGTACGCATGGAACTGTGTGACGTACCCTCGTGTAGTCCCTAG
SEQ ID NO.21: Nucleotide sequence encoding the kringle domain of the kremen
protein
CCCGAGTGTTTCACAGCCAATGGTGCGGATTATAGGGGAACACAGAACTGGACAGCACTACAAGGCGGG
AAGCCATGTCTGTTTTGCAACGAGACTTTCCAGCATCCATACAACACTCTGAAATACCCCAACGGGGAG
GGGGCCCTGGGTGAGCACAACTATTGCAGAAATCCAGATGGAGACGTGAGCCCCTGGTGCTATGTCGCA
GAGCACGAGGATGGTGTCTACTGGAAGTACTGTGAGATACCTGCTTGCCAGATGTAG
SEQ ID NO.22: Nucleotide sequence encoding the kringle domain of the t-PALP
GGAGGCTGTTTCTGGGACAACGGCCACCTGTACCGGGAGGACCAGACCTCCCCCGCGCCGGGCCTCCGC
TGCCTCAACTGGCTGGACGCGCAGAGCGGGCTGGCCTCGGCCCCCGTGTCGGGGGCCGGCAATCACAGT
TACTGCCGAAACCCGGACGAGGACCCGCGCGGGCCCTGGTGCTACGTCAGTGGCGAGGCCGGCGTCCCT
GAGAAACGGCCTTGCGAGGACCTGCGCTGTCCAGAGTAG
SEQ ID NO.23: Nucleotide sequence encoding the kringle domain of the RGD
receptor kinase
CTGGCCTGCTCGCATCCCTTCTCGAAGAGTGCCACTGAGCATGTCCAAGGCCACCTGGGGAAGAAGCAG
GTGCCTCCGGATCTCTTCCAGCCATACATCGAAGAGATTTGTCAAAACCTCCGAGGGGACGTGTTCCAG
AAATTCATTGAGAGCGATAAGTTCACACGGTTTTCCCAGTGGAAGAATGTGGAGCTCAACATCCACCTG
ACCATGAATGACTTCAGCGTGCATCGCATCATTGGGCGCGGGGGCTTTGGCGAGGTCTATGGGTGCCCG
AAGTAG
SEQ ID NO.24: Nucleotide sequence encoding the kringle domain of ApoArgC
CAGGAGTGCTACCACAGTAATGGACAGAGTTATCGAGGCACATACTTCACCACTGTCACACGAAGAACC
TGCCAAGCTTGGTCATCTATGACGCCACACCAGCACAGTAGAACCCCAGAAAAGTACCCAAATGATGGC
TTGATCTCGAACTACTGCAGGAATCCGGATGGTTCGGCAGGCCCTTGGTGTTATACGACGGATCCCAAT
GTCAGGTGGGAGTACTGCAACCTGACACGGTGCTCAGACTAG
SEQ ID NO.25: Nucleotide sequence encoding the kringle domain 1 of the
macrophage stimulating protein
CGGACCTGCATCATGAACAATGGGGTTGGGTACCGGGGCACCATGGCCACGACCGTGGGTGGCCTGCCC
TGCCAGGCTTGGAGCCACAAGTTCCCGAATGATCACAAGTACACGCCCACTCTCCGGAATGGCCTGGAA
GAGAACTTCTGCCGTAACCCTGATGGCGACCCCGGAGGTCCTTGGTGCTACACAACAGACCCTGCTGTG
CGCTTCCAGAGCTGCGGCATCAAATCCTGCCGGGAGTAG
SEQ ID NO.26: Nucleotide sequence encoding the kringle domain 2 of the
macrophage stimulating protein
GCCGCGTGTGTCTGGGGCAATGGCGAGGAATACCGCGGCGCGGTAGACCGCACGGAGTCAGGGCGCGAG
TGCCAGCGCTGGGATCTTCAGCACCCGCACCAGCACCCCTTCGAGCCGGGCAAGTTCCTCGACCAAGGT
CTGGACGACAACTATTGCCGGAATCCTGACGGCTCCGAGCGGCCATGGTGCTACACTACGGATCCGCAG
ATCGAGCGAGAGTTCTGTGACCTCCCCCGCTGCGGGTCCTAG
SEQ ID NO.27: Nucleotide sequence encoding the kringle domain 3 of the
macrophage stimulating protein
GTCAGCTGCTTCCGCGGGAAGGGTGAGGGCTACCGGGGCACAGCCAATACCACCACTGCGGGCGTACCT
TGCCAGCGTTGGGACGCGCAAATCCCGCATCAGCACCGATTTACGCCAGAAAAATACGCGGGCAAAGAC
CTTCGGGAGAACTTCTGCCGGAACCCCGACGGCTCAGAGGCGCCCTGGTGCTTCACACTGCGGCCCGGC
ATGCGCGCGGCCTTTTGCTACCAGATCCGGCGTTGTACAGACTAG
SEQ ID NO.28: Nucleotide sequence encoding the kringle domain 4 of the
macrophage stimulating protein
CAGGACTGCTACCACGGCGCAGGGGAGCAGTACCGCGGCACGGTCAGCAAGACCCGCAAGGGTGTCCAG
TGCCAGCGCTGGTCCGCTGAGACGCCGCACAAGCCGCAGTTCACGTTTACCTCCGAACCGCATGCACAA
CTGGAGGAGAACTTCTGCCGGAACCCAGATGGGGATAGCCATGGGCCCTGGTGCTACACGATGCACCCA
AGGACCCCATTCGACTACTGTGCCCTGCGACGCTGCGCTGATTAG

The polypeptide sequences of the various human abrogens having sequences of SEQ ID NOs: 1-14 fused to the IL-2 signal peptide and to human serum albumin or immunoglobulin IgG2 Fe region, as well as linker peptide sequences are listed below.

SEQ ID NO: 29
AKTCYEGNGH FYRGKASTDT MGRPCLPWNS ATVLQQTYHA HRSDALQLGL
GKHNYCRNPD NRRRPWCYVQ VGLKPLVQEC MVHDCAD
SEQ ID NO: 30
AKTCYEGNGH FYRGKASTDT MGRPCLPWNS ATVLQQTYHA HRSNALQLGL
GKHNYCRNPD NRRRPWCYVQ VGLKPLVQEC MVHDCAD
SEQ ID NO: 31
DAHKSEVAH RFKDLGEENF KALVLIAFAQ YLQQCPFEDH VKLVNEVTEF
AKTCVADESA ENCDKSLHTL FGDKLCTVAT LRETYGEMAD CCAKQEPERN
ECFLQHKDDN PNLPRLVRPE VDVMCTAFHD NEETFLKKYL YEIARRHPYF
YAPELLFFAK RYKAAFTECC QAADKAACLL PKLDELRDEG KASSAKQRLK
CASLQKFGER AFKAWAVARL SQRFPKAEFA EVSKLVTDLT KVHTECCHGD
LLECADDRAD LAKYICENQD SISSKLKECC EKPLLEKSHC IAEVEMDEMP
ADLPSLAADF VESKDVCKNY AEAKDVFLGM FLYEYARRHP DYSVVLLLRL
AKTYETTLEK CCAAADPHEC YAKVFDEFKP LVEEPQNLIK QNCELFEQLG
EYKFQNALLV RYTKKVPQVS TPTLVEVSRN LGKVGSKCCK HPEAKRMPCA
EDYLSVVLNQ LCVLHEKTPV SDRVTKCCTE SLVNPRPCFS ALEVDETYVP
KEFNAETFTF HADICTLSEK ERQIKKQTAL VELVKHKPKA TKEQLKAVMD
DFAAFVEKCC KADDKETCFA EEGKKLVAAS QAALGL
SEQ ID NO: 32
DAGGGGSGGGGSGGGGS
SEQ ID NO: 33
ADAHKSEVAH RFKDLGEENF KALVLIAFAQ YLQQCPFEDH VKINNEVTEF
AKTCVADESA ENCDKSLHTL FCDKLCTVAT LRETYGEMAD CCAKQEPERN
ECFlQHKDDN PNLPRLVRPE VDVMCTAFHD NEETFLKKYL YEIARRHPYF
YAPELLFFAK RYKAAFTECC QAADKAACLL PKLDELRDEG KASSAKQRLK
CASLQKFGER AFKAWAVARL SQRFPKAEFA EVSKLVTDLT KVHTECCHGD
LLECADDRAD LAKYICENQD SISSKLKECC EKPLLEKSHC IAEVENDEMP
ADTPSLAADF VESKDVCKNY AEAKDVFLGM FLYEYARRHP DYSVVLJLRL
AKTYETTLEK CCAAADPHEC YAKVFDEFKP LVEEPQNLIK QNCELFEQLG
EYKFQNALLV RYTKKVPQVS TPTLVEVSRN LGKVGSKCCK HPEAKRMPCA
EDYLSVVLNQ LCVLHEKTPV SDRVTKCCTE SLVNRRPCFS ALEVDETYVP
KEFNAETFTF HADICTLSEK ERQIKKQTAL VELVKHKPKA TKEQLKAVMD
DFAAFVEKCC KADDKETCFA EEGKKLVAAS QAALGLDAGG GGSGGGGSGG
GGSKTCYEGN GHFYRGKAST DTMGRPCLPW NSATVLQQTY HAHRSNALQL
GLGKHNYCRN PDNRRRPWCY VQVGLKPLVQ ECMVHDCAD
SEQ ID NO: 34
ADAHKSEVAI RFKDLGEENF KALVLIAFAQ YLQQCPFEDH VKLVNEVTEF
AKTCVADESA ENCDKSLHTL FGDKLCTVAT LRETYGEMAD CCAKQEPERN
ECFLQHKDDN PMLPRLVRPE VDVMCTAFHD NEETFLKKYL YEIARRHPYF
YAPELLFFAK RYKAAFTECC QAADKAACLL PKLDETRDEG KASSAKQRLK
CASLQKFGER AFKAWAVARL SQRFPKAEFA EVSKLVTDLT KVHTECCHGD
LTECADDRAD LAKYLCENQD STSSKLKECC EKPLLEKSHC IAEVENDEMP
ADLPSLAADF VESKDVCKNY AEAKDVFLGM FLYEYARRHP DYSVVLLLRL
AKTYETTLEK CCAAADPHEC YAKVFDEFKP LVEEPQNLTK QNCELFEQLG
EYKFQNALLV RYTKKVPQVS TPTLVEVSRN LGKVGSKCCK HPEAKRMPCA
EDYLSVVLNQ LCVLHEKTPV SDRVTKCCTE SLVNRRPCFS ALEVDETYVP
KEFNAETFTF HADICTLSEK ERQIKKQTAL VELVKHKPKA TKEQLKAVMD
DFAAFVEKCC KADDKETCFA EEGKKLVAAS QAALGLDAKT CYEGNGHFYR
GKASTDTMGR PCLPWNSATV LQQTYHAHRS NALQLGLGKH NYCRNPDNRR
RPWCYVQVGL KPLVQECMVH DCAD
SER ID NO: 35
AKTCYEGNGH FYRGKASTDT MGRPCLPWNS ATVLQQTYHA HRSNALQLGL
GKHNYCRNPD NRRRPWCYVQ VCLKPLVQEC MVHDCADDAH KSEVAHRFKD
LGEENFKALV LIAFAQYLQQ CPFEDHVKLV NEVTEFAKTC VADESAENCD
KSLHTLFGDK LCTVATLRET YGEMADCCAK QEPERNECEL QHKDDNPNLP
RLVRPEVDVM CTAFHDNEET FLKKYLYEIA RRHPYFYAPE LLFFAKRYKA
AFTECCQAAD KAACLLPKLD ELRDEGKAS SAKQRLKCASL QKFGEPAFKA
WAVARLSQRF PKAEFAEVSK LVTDLTKVHT ECCHGDLLEC ADDRADLAKY
ICENQDSISS KLKECCEKPL LEKSHCIAEV ENDEMPADLP SLAADFVESK
DVCKNYAEAK DVFLGMFLYE YARRHPDYSV VLLLRLAKTY ETTLEKCCAA
ADPHECYAKV FDEFKPLVEE PQNLTKQNCE LFEQLGEYKF QNALLVRYTK
KVPQVSTPTL VEVSRNLGKV GSKCCKHPEA KRMPCAEDYL SVVLNQLCVL
HEKTPVSDRV TKCCTESLVN RRPCFSALEV DETYVPKEFN AETFTFHADI
CTLSEKERQI KKQTALVELV KHKPKATKEQ LKAVMDDFAA FVEKCCKADD
KETCFAEEGK KLVAASQAAL CL
SEQ ID NO: 36
GGGGSGGGGSGGGGS
SEQ ID NO: 37
AKTCYEGNGH FYRGKASTDT MGRPCLPWNS ATVLQQTYHA HRSNALQLGL
CKHNYCRNPD NRRRPWCYVQ VCLKPLVQEC MVHDCADGGG GSGGGGSGGG
GSDAHKSEVA HRFKDLGEEN FKALVLIAFA QYLQQCPFED HVKLVNEVTE
FAKTCVADES AENCDKSTHT LFGDKLCTVA TLRETYGEMA DCCAKQEPER
NECFLQHKDD NPNLPRLVRP EVDVMCTAFH DNEETFLKKY LYEIAPRHPY
FYAPELLFFA KRYKAAFTEC CQAADKAACL LPKLDELRDE GKASSAKQRL
KCASLQKFGE RAFKAWAVAR LSQRFPKAEF AEVSKLVTDL TKVHTECCHG
DLLECADDRA DLAKYICENQ DSISSKLKEC CEKPLLEKSH CIAEVENDEM
PADLPSLAAD FVESKDVCKM YAEAKDVFLG MFLYEYARRH PDYSVVLLLR
LAKTYETTLE KCCAAADPHE CYAKVFDEFK PLVEEPQNLT KQNCELFEQL
GEYKFQNALL VRYTKKVPQV STPTLVEVSR NLGKVGSKCC KHPEAKRMPC
AEDYLSVVLN QLCVLHEKTP VSDRVTKCCT ESLVNRRPCF SALEVDETYV
PKEFNAETFT FHADICTLSE KERQIKKQTA LVELVKHKPK ATKEQLKAVM
DDFAAFVEKC CKADDKETCF AEEGKKLVAA SQAALGLJ
SEQ ID NO: 38
DAHKSEVAHR FKDLGEENFK ALVLIAFAQY LQQCPFEDHV KLVNEVTEFA
KTCVADESAE NCDKSLHTLF GDKLCTVATL RETYGEMADC CAKQEPERNE
CFLQHKDDNP NLPRLVRPEV DVMCTAFHDN EETFLKKYLY EIAPRHPYFY
APELLFFAKR YKAAFTECCQ AADKAACLLP KLDELRDEGK ASSAKQRLKC
ASLQKFGERA FKAWAVARLS QRFPKAEFAE VSKLVTDLTK VHTECCHGDL
TECADDRADL AKYICENQDS ISSKLKECCE KPLLEKSHCI AEVENDEMPA
DLPSLAADFV ESKDVCKNYA EAKDVFLGMF LYEYARRHPD YSVVLLLRLA
KTYETTLEKC CAAADPHECY AKVFDEFKPL VEEPQNLIKQ NCELFEQLGE
YKFQNALLVR YTKKVPQVST PTLVEVSRNL GKVGSKCCKH PEAKRMPCAE
DYLSVVLNQL CVLHEKTPVS DRVTKCCTES LVNRRPCFSA LEVDETYVPK
EFNAETFTFH ADICTLSEKE RQIKKQTALV ELVKHKPKAT KEQLKAVMDD
FAAFVEKCCK ADDKETCFAE EGKKLVAASQ AALGLDAGGG GSGGGGSGGG
GSKTCYEGNG HFYRGKASTD TMGRPCLPWN SATVLQQTYH AHRSNALQLG
LGKHNYCRNP DNRRRPWCYV QVGLKPLVQE CMVHDCAD
SEQ ID NO: 39
EPRGPTIKPCPPCKCPAPNLLGGPSVFTFPPKIKDVLMISLSPIVTCVVVDVSED
DPDVQISWFVNNVEVHTAQTQTHREDYNSTLRVVSALPIQHQDWMSGKEFKCKVN
NKDLPAPIERTISKPKGSVRAPQVYVLPPPEEEMTKKQVTLTCMVTDFMPEDIYV
EWTNNGKTELNYKNTEPVLDSDGSYFMYSKLRVEKKNWVERMSYSCSVVHEGLHN
HHTTKSFSRTPGK
SEQ ID NO: 40
ARLEPRGPTI KPCPPCKCPA PNLLGGPSVF IFPPKIKDVL MISLSPIVTC
VVVDVSEDDP DVQISWFVNN VEVHTAQTQT HREDYNSTLR VVSALPIQHQ
DWMSGKEFKC KVNNKDLPAP IERTISKPKG SVRAPQVYVL PPPEEEMTKK
QVTLTCMVTD FMPEDIYVEW TNNGKTELNY KNTEPVLDSD GSYFMYSKLR
VEKKNWVERN SYSCSVVHEG LHNHHTTKSF SRTPGKKTCY EGNGHFYRGK
ASTDTMGRPC LPWNSATVLQ QTYHAHRSNA LQLGLGKHNY CRNPDNRRRP
WCYVQVGLKP LVQECMVHDC AD
SEQ ID NO: 41
AKTCYEGNGH FYRGKASTDT PGRPCLPWNS ATVLQQTYHA HRSNALQLGL
GKHNYCRNPD NRRRPWCYVQ VGLKPLVQEC MVHDCADRLE PRGPTIKPCP
PCKCPAPNLL GGPSVFIFPP KIKDVLMISL SPIVTCVVVD VSEDDPDVQI
SWFVNNVEVH TAQTQTHRED YNSTLRVVSA LPIQHQDWMS GKEFKCKVNN
KDLPAPIERT ISKPKGSVRA PQVYVLPPPE EEMTKKQVTL TCMVTDFMPE
DIYVEWTNNG KTELNYKNTE PVLDSDGSYF MYSKLRVEKK NWVERNSYSC
SVVHEGLHNH HTTKSFSRTP GK
SEQ ID NO: 42: Kringle K4 of Plasminogen
HMAQDCYH GDGQSYRGTS STTTTGKKCQ SWSSMTPHRH QKTPENYPNA
GLTMNYCRNP DADKGPWCFT TDPSVRWEYC NLKKCSG
SEQ ID NO: 43: Kringle K5 of Plasminogen
HMEEDCMF GNGKGYRGKR ATTVTGTPCQ DWAAQEPHRH SIFTPETNPR
AGLEKNYCRN PDGDVGGPWC YTTNPRKLYD YCDVPQCAA

The cDNA sequence can be obtained from GenBank or a number of available sources.

PCR based methods can be used to retrieve the cDNA from an appropriate library. The cDNA can then be conveniently stored in a vector such as the pGEM or pGEX vectors by standard ligation or plasmid manipulation methods. The polypeptide encoding regions are then transferred into an appropriate, selected expression cassette or vector. Specific examples of vectors for various applications exist, including gene therapy (Chen et al., Hum Gen Ther 11: 1983-96 (2000); MacDonald et al., Biochem Biophys Res Comm 264:469-477 (1999); Cao et al., J Biol Chem 271:29461-67 (1996); Li et al., Hum Gene Ther 10:3045-53 (1999)). For the examples that follow, the method of Soubrier et al., Gene Therapy 6:1482-1488 (1999), is used to prepare recombinant adenovirus with E1/E3 deletion, CMV expression promotor and SV40 polyA. The plasmid vector used below contains the Amp resistance gene, the CMV promotor, the SV40 poly A sequence, and the IL-2 signal sequence for efficient secretion. The fairly robust adenoviral system can be selected for its ability to be used in a variety of cell types, whereas the plasmid system is selected for its relative efficiency of vector introduction. One skilled in the art is familiar with selecting or modifying vectors with these or other elements for use.

Once cloned and inserted into an appropriate vector, any of the abrogen encoding sequences or abrogen derivatives encoding sequences can be assayed for specific activity related to anti-angiogenesis using the Examples below or an assay mentioned here or in the references.

In a preferred embodiment for expressing a recombinant abrogen polypeptide, a vector comprising the coding region for human serum albumin linked to the C-terminus of the abrogen encoding region is used (see, for example, Lu et al., FEBS Lett. 356: 56-9 (1994)). Other fusion proteins or chimeric proteins can also be used. In another embodiment of a fusion protein, the abrogen encoding region is linked to an immunogenic peptide or polypeptide encoding region. These fusions can be used in created antibodies or monoclonal antibodies against an abrogen. Methods for preparing antibodies are well known in the art and both the purified abrogen polypeptides and fusion of them can be used to prepare antibodies. Monoclonal antibodies can be prepared using hybridoma technology (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., pp. 563-681 (1981)). In general, such procedures involve immunizing an animal (preferably a mouse) with polypeptide or, more preferably, with a secreted polypeptide expressing cell. The mice splenocytes are extracted and fused with a suitable myeloma cell line, such myeloma cell line SP20, available from the ATCC. After fusion, the resulting hybridoma cells are selectively maintained in HAT medium and then cloned by limiting dilution as described (Wands et al., Gastroenterology 80:225-232 (1981)).

The hybridoma cells obtained through such a selection are then assayed to identify clones, which secrete antibodies capable of binding the polypeptide. Additional fusions can be used to ease purification of abrogen polypeptides, including poly-His tracks, constant domain of immunoglobulins (IgG), the carboxy terminus of either Myc or Flag epitope (Kodak), and glutathione-S-transferase (GST) fusions. Plasmids for this purpose are readily available.

A relatively simple method for preparing recombinant or purified abrogen polypeptide involves the baculovirus expression system or the pGEX system (Nesbit et al., Oncogene 18:6469-6476 (1999), Nesbit et al., J of Immunol 166:6483-90 (2001)). In the baculovirus system, plasmid DNA encoding the abrogen polypeptide is cotransfected with a commercially available, linearized baculovirus DNA (BaculoGold baculovirus DNA, Pharmingen, San Diego, Calif.), using the lipofection method (Felgner et al., PNAS 84:7413-7417 (1987)). BaculoGold virus DNA and the plasmid DNA are mixed in a sterile well of a microtiter plate containing 50 μl of serum-free Grace's medium (Life Technologies Inc., Gaithersburg, Md.). 10 μl Lipofectin and 90 μl Grace's medium are added, mixed and incubated for 15 minutes at room temperature.

The transfection mixture is added drop-wise to Sf 9 insect cells (ATCC CRL 1711), and seeded in a 35 mm tissue culture plate with 1 ml Grace's medium without serum. The plate is then incubated for 5 hours at 27° C. The transfection solution is then removed from the plate and 1 ml of Grace's insect medium supplemented with 10% fetal calf serum is added. The cells are cultured at 27° C. for four days. The cells can then be selected for appropriately transduction and assayed for the expression of abrogen polypeptide. If a fusion polypeptide was desired, the fusion polypeptide can be purified by known techniques and used to prepare monoclonal antibodies.

### Example 2

### Proliferation Analysis of Transduced HUVEC Using Alamar Blue.

A number of different assays for analyzing cell proliferation, tubule formation, cell migration, endothelial cell growth, and tumor metastasis exist. Some of them are described in the references cited.

Human umbilical vein endothelial cells (HUVEC: Clonetics, San Diego) are seeded at 5×10⁵ cells/well of 6-well-plate in EGM-2 medium. The cells are incubated overnight at 37° C., 5% CO₂. Endothelial Cell Basal Medium (EBM) and Endothelial Cell Growth Medium (EGM) are available (Clonetics, San Diego). The medium is aspirated off and 500 μl of ECM medium containing 100 IT/cell viruses put over cells. The cells are incubated at 37° C. for 2 hours, then aspirated and 1.5 ml EGM-2 medium is added. The cells are again incubate overnight at 37° C.

The cells are trypsinized, counted, and seeded at 2000cell/well of 96-well-plate in EGM-2 medium. The cells are incubated at 37° C. for 3 hours. The medium is changed into 200 μl of the following medium: Control=ECM+0.5% FBS; Test 1=control medium with bFGF 10 ng/ml; Test 2=control medium with VEGF 10 ng/ml; Test 3=control medium with bFGF 10 ng/ml+VEGF 10 ng/ml. After changing the medium, the cells are incubated at 37° C. for 5 days. 201 μl Alamar Blue (BioSource International) for each well is added. Plates are incubated at 37° C. for 6 hours and then the OD read at 570 nm and 595 nm.

This proliferation assay of abrogen polypeptides (SEQ ID NO.: 1-14) can show the effectiveness of the polypeptides according to the present invention in abrogating the proliferation of endothelial cells induced by bFGF and VEGF.

### Example 3

### Assay of Transduced HUVEC Embedded in Fibrin gel

In an assay that distinguishes the abrogen activity from angiostatin, human umbilical vein endothelial cells (HUVEC: Clonetics, San Diego) are seeded (passage 3, growing in EGM-2 medium) at 5×10⁵ cells/well of 6-well-plate in EGM-2 medium. The embedded cell assay also or alternatively provides data concerning the invasiveness of the endothelial cells in response to certain treatments. Endothelial cell tubule formation induced by pro-angiogenic factors such as FGF and VEGF, a characteristic measured by this assay, can be directly correlated to angiogenesis. The abrogen activity inhibits or reduces angiogenesis by inhibiting tubule formation. The use of virally transduced HUVEC can provide very detailed information as to the effects that a selected abrogen polypeptide or derivative has on primary cell types. The potential anti-angiogenic agents are introduced by transduction of the cells using a recombinant human adenovirus.

The fibrin gel includes PBS (control), VEGF or bFGF. HUVEC cells are split ½ to

⅓ the day before transduction. On the day of the transduction, the cells are washed with PBS. 10 ml of serum free medium containing 100:1 (IT: cell ratio) of virus is incubated with the HUVEC for 2 hours to transduce the cells. The medium is then removed and the cells washed with PBS and 20 ml of full HUVEC medium placed in each T150 flask.

48 hours following transduction the cells are trypsinized and the concentration of each cell solution adjusted to 5×10⁵ cell/ml. The assay is performed in a 24 well plate. Each well is coated with 200 μl of fibrinogen solution (12 mg/ml) and 8 μl of thrombin (50U/ml). Then in each well is added (according to the conditions):

VEGF165 (2 μl), b-FGF(2 μl) or nothing (final [growth factor]=1 ug/ml)

Thrombin (20ul) of a 1000 U/ml solution.

250 μl cell solution for a final concentration of 5×10⁵ cells/ml

250 μl of fibrinogen

Gels set in about 30 seconds. Then, 1.5 ml of medium is added on top. Each type of infected cells was assayed with VEGF165 alone, b-FGF alone or without any growth factor other than those already present in the medium.

After 6 days medium is removed and cells subjected to staining with Dif-Quick for enhanced visualization under microscopy. Fibrin plugs are fixed in 10% formalin, and then subjected to the 3 Dif Quick stains for 15 mins each before being rinsed in PBS and then fixed with 10% formalin again.

Tubule formation can be correlated with endothelial cell invasiveness, a characteristic of angiogenic activity. Thus, the lack of tubule formation in the abrogen polypeptides samples demonstrate an inhibtion of endothelial cell invasiveness, correlating to an inhibition of angiogenesis and metastasis.

### Example 4

### In Vivo Expression of Abrogen Polypeptides Using Adenoviral Vectors.

For in vivo documentation of the activity of abrogen, a first experiment involves the systemic injection iv of 1×10¹¹ VP of adenovirus containing nucleic acid of SEQ ID NO: 15-28. Circulating levels of the abrogen polypeptides is measured by Western. Exemplary expression levels at d4 can be between 500-1000 ng/ml in either SCID or SCID/Beige mice. The 4T1 spontaneously metastatic breast cell line in SCID mice is used in which animals are injected with 2×10⁵ cells sub-cutaneously in the right flank. At d7, when tumors were 20-40 mm³, adenovirus is injected at 1×10¹¹ VP: Tris, CMV1.0 control Ad. A second and third iv administration of adenovirus can be performed. Lung metastasis is then measured at about day 35.

### Example 5

### In Vivo Expression of Abrogen Polypeptides Using Plasmid Vectors.

Two tumor models are used, employing 4T1 tumor cells and 3LL Boston tumor cells. In the assay, the anti-tumor activity of abrogen polypeptides in the prophylactic murine Lewis lung carcinoma model, 3LL-B, in C57BL/6 mice is tested. The assay is designed to assess whether circulating levels of abrogen polypeptides prevent and/or reduce the formation and growth of spontaneously formed metastases from subcutaneously implanted primary tumors. The tumor cells are cultured in DMEM containing 10% FCS, sodium pyruvate, nonessential amino acids, Pen-Strep, and L-Glutamine until prepared for injection using a buffered saline solution. The tumor cells are injected into the right flank of 8-10 week old C57BL/6 or BALB/c female mice via subcutaneous injection of a suspension of 2.5×10⁵ tumor cells. Six days prior to tumor cell injection, the 25 ul of the plasmid solutions (25 ug DNA in Tris EDTA with 10% glycerol) are injected into the tibialis cranialus muscle. The injection site is then exposed to 4 pulses (1 pulse per second) at 100 mV using a square wave pulse generator (the electrotransfer method, ET). Alternatively, the electrotransfer enhancement can utilize four electric pulses of 100 V (250 V/cm) at 1 Hz with a pulse length of 20 msec. On about day 15 post cell injection, the primary subcutaneous tumor was surgically removed. At day 35, the lungs are collected and tumor nodules measured. Expression levels are measured on day-1, 7, and 14 relative to electrotransfer. A control alkaline phosphatase expressing plasmid (mSEAP; see, for example, WO 02/095068) is used to assay expression.

The reduction of the size and number of metastasis is then measured and compared with control plasmid and known anti-angiogenic polypeptides endostatin and angiostatin.

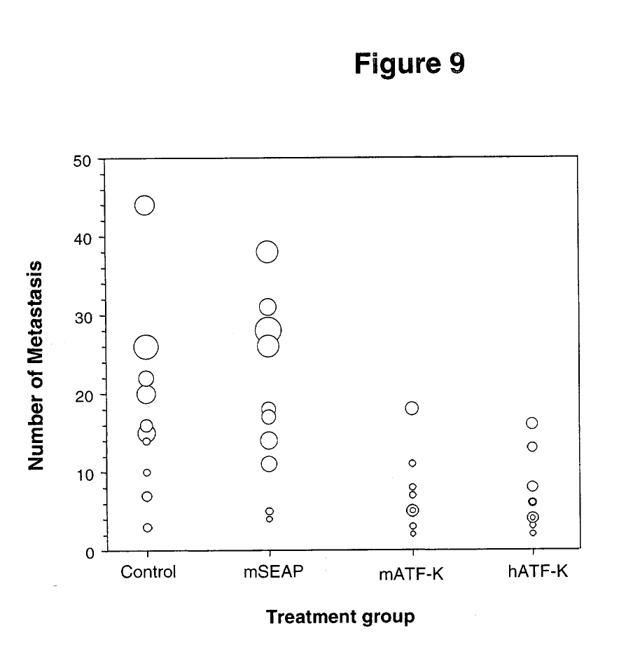

Another set of assays with 3-LL Boston cells employing electrotransfer enhancement with four electric pulses of 100 V (250 V/cm) at 1 Hz with a pulse length of 20 msec are shown in FIG. 11. Metastases are counted using a dissecting microscope.

To assess the anti-tumor activity of systemically expressed abrogen polypeptides in a human breast adenocarcinoma xenograft model of SCID/bg mice, MDA-MB-435 tumor cells are used. These cells are significantly less aggressive as compared to the 4T1 and 3LL-B syngeneic mouse tumor models. However, spontaneous lung metastases formation is established in the time frame of 35 days post subcutaneous cell injection. Subcutaneous palpable MDA-MB-435 tumors are established by injecting SCID/bg mice with 106 tumor cells. On day 10 post injection, plasmid DNA was transferred to the Tibialis cranialis muscle using electrotransfer as described previously. Briefly, 25 μg of plasmid DNA (a total of 50 μg) in a 25 μl volume are injected directly into each T. cranialis muscle followed by four electric pulses of 100 V (250 V/cm) at 1 Hz with a pulse length of 20 msec. The primary tumor is carefully removed when the volume reached between 250 and 350 mm3, i.e. on day 39 or 44 post cell injections depending on the growth of the primary tumor. The study is terminated on day 89 and lungs harvested carefully and fixed in Bouin's solution. Metastases are counted using a dissecting microscope.

### Example 6

### Production of Derivative Abrogen Polypeptides by PCR Based Site-Directed Mutagenesis.

In one method for generating an abrogen derivative, four oligonucleotide primers are used. Two of these are primers that flank the ends of the cDNA (SEQ ID NO.: 15-28) and contain convenient restriction sites for cloning into a desired vector. The other two mutagenic primers are complementary and contain the mutation(s) of interest. Typically, the mutagenic primers overlap by about 24 base pairs. Two separate PCR reactions are performed, each using a different outside primer and a different mutagenic primer that anneal to opposite strands of the DNA template. The amplified product from both PCR reactions are purified and added to a new primerless PCR mix.

After a few PCR cycles, the two products are annealed and extended at the region of overlap yielding the derivative product. The two outside primers are then added to this mixture to amplify the cDNA product by PCR. This method can be used to introduce amino acid substitutions at any point in an abrogen sequence.

In addition to the conservative amino acid substitutions noted throughout the disclosure, one skilled in the art is familiar with numerous methods for analyzing and selecting homologs and derivative sequences to use as abrogen sequences. For example, the sequence identified as “Putative-K1 (Est)” in FIG. 2 can be identified by searching for homologs using GenBank, an EST database, or any cDNA or genomic DNA database available. The EST can be pulled from a library, PCR amplified using primers specific for the EST, or synthesized using automated methods. Once isolated, the polypeptide encoding region can be cloned into an appropriate vector and tested as described above. As noted above, additional sequences can be used to produce the kringle polypeptides and abrogen activity of the invention and additional species can be used as well. For example, any of the proteins listed herein or any other available kringle-containing proteins can be selected for use, such as factor XII, hepatocyte growth factor activator (HGFA), hyaluronan binding protein, neurotrypsin, retinoic acid-related receptors 1 and 2 (ROR-1 and ROR-2), the kremen protein, tissue-type plasminogen activator protease (t-PALP), apolipoprotein ArgC, macrophage stimulating proteins (MSP), and thrombin.

### Example 7

### Construction of IL2 sp-Abrogen Polypeptide

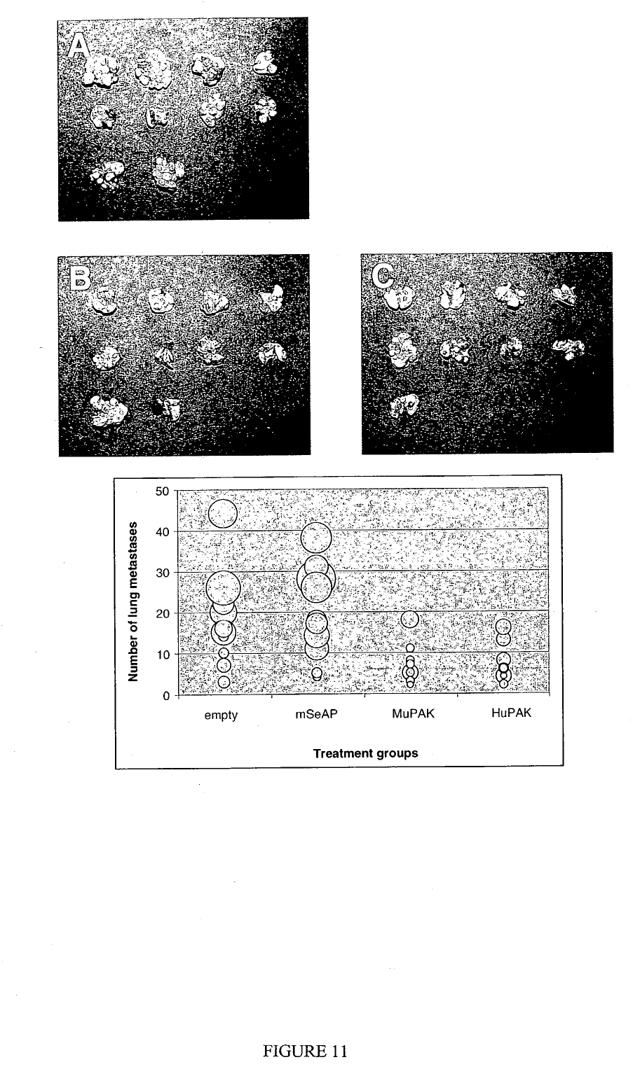

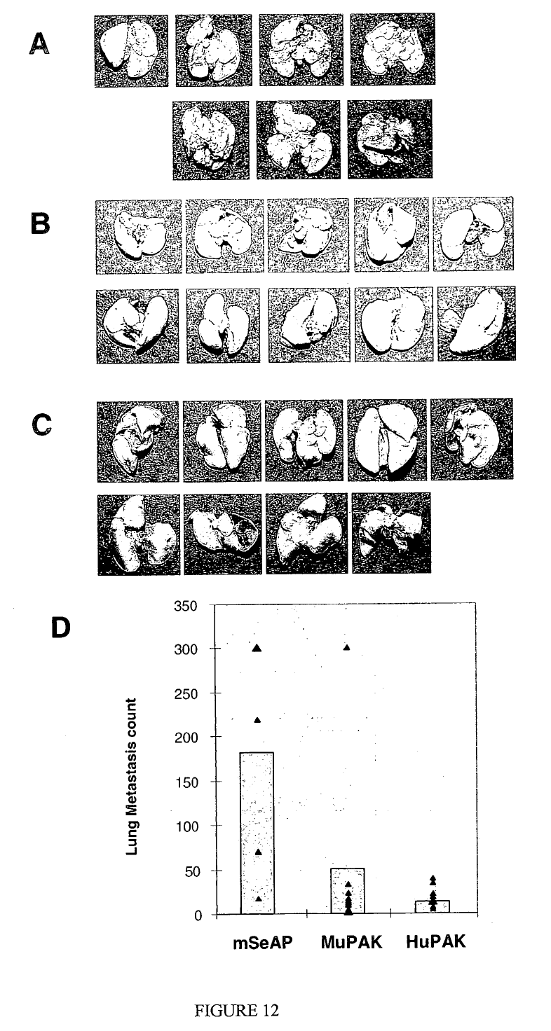

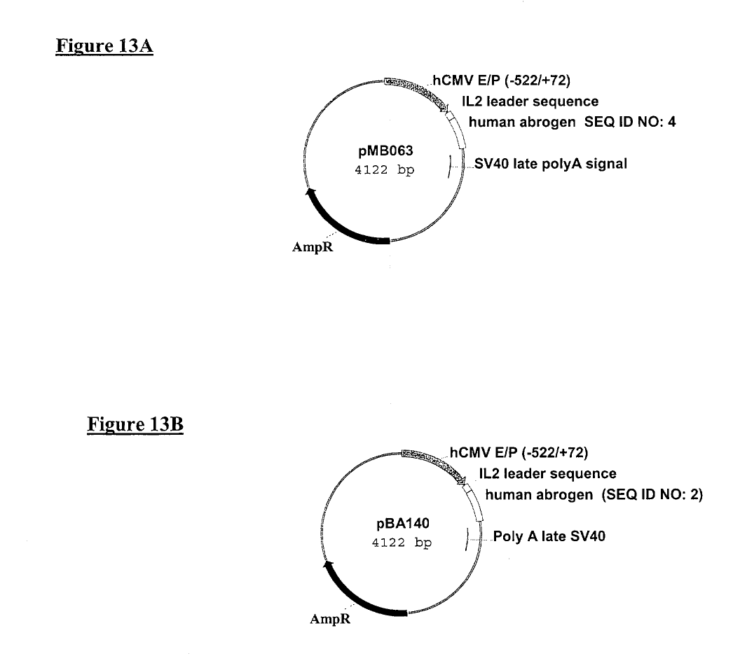

The combined techniques of site-directed mutagenesis and PCR amplification allowed to construct a chimeric gene encoding a chimeric peptide resulting from the translational coupling between the first 20 amino acids of the interleukin 2 signal peptide, which represent a signal sequence or signal peptide that is cleaved to produce the mature factor (Tadatsugu, T. et al. (1983) Nature 302:305) and the abrogen sequences as set forth in SEQ ID NO: 4 (IL2sp-abrogen). These hybrid genes were preferably bordered in 5′ of the translational initiator ATG and in 3′ of the translational stop codon and encode chimeric proteins of the IL2sp-abrogen. The hybrid gene is cloned in the pXL2996 (FIG. 13A), under the control of the human CMV Enhancer/promoter (−522/+72) and upstream of a SV40 late poly A signal. The resulting plasmid pMB063 as described in FIG. 13A was obtained. The abrogen peptide secreted from the plasmid pMB063 retained an alanine from the IL-2 signal peptide at the N-terminus, and thus contains a 87 amino acid sequence as set forth in SEQ ID NO: 9.

The hybrid nucleotide sequence comprising the interleukin 2 signal peptide sequence and the abrogen sequence as set forth in SEQ ID NO: 2 was cloned in plasmid pXL 2996 downstream of the human CMV enhancer/promoter (−522/+72) and upstream of a SV40 late poly A signal. The resulting plasmid pBA140 as described in FIG. 13B was obtained. The abrogen peptide secreted from the plasmid pBA140 also retained an alanine from the IL-2 signal peptide at the N-terminus, and thus contains a 87 amino acid sequence as set forth in SEQ ID NO: 10.

### Example 8

### Construction of Fusion Proteins of Abrogen and HSA

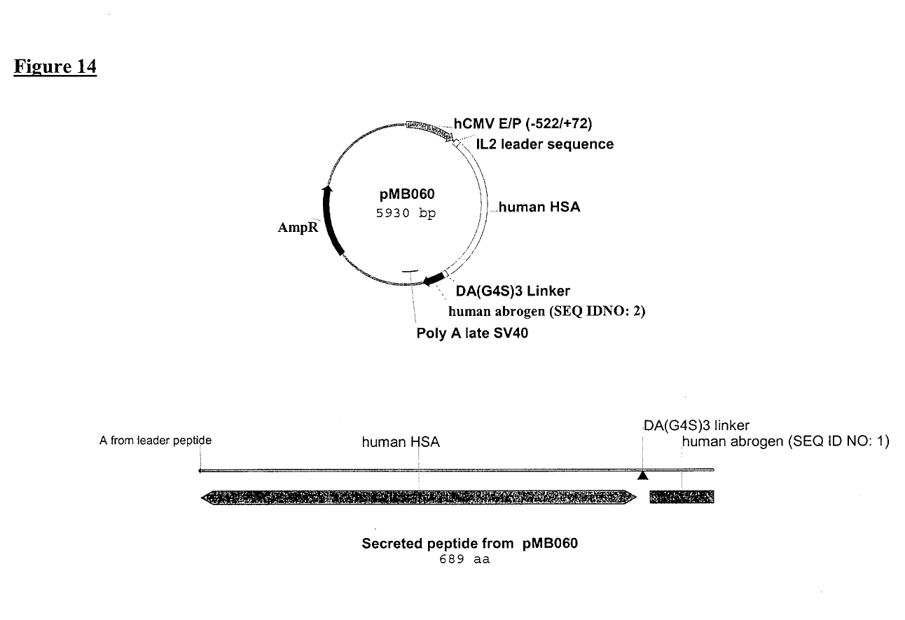

A nucleotide fragment containing from 5′ to 3′ the IL-2 signal peptide, the nucleotide sequence encoding the human HSA as set forth in SEQ ID NO: 11, a linker, and the abrogen sequence as set forth in SEQ ID NO: 2 was cloned in plasmid pXL2996 downstream to the human CMV promoter and upstream of a SV40 polyA. The linker DA(G₄S)₃ was used (SEQ ID NO: 32). The construct of the fusion protein IL2sp-HSA-linker-abrogen and the resulting plasmid designated pMB060 are shown in FIG. 14. The fusion protein HSA/abrogen secreted from the plasmid pMB060 has the sequence as set forth in SEQ ID NO: 13.

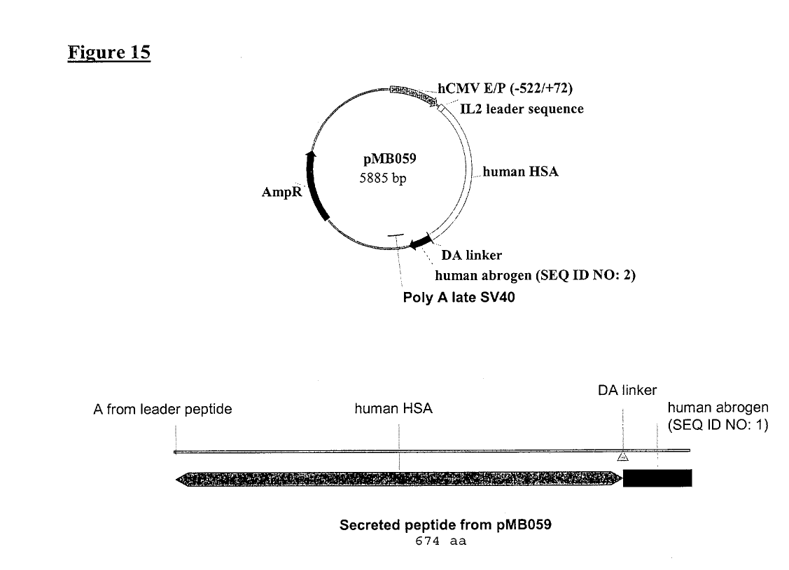

Another linker DA (Asp-Ala) was used. The chimeric construct of the fusion protein IL2sp-HSA-DA linker-abrogen and the resulting plasmid is designated pMB059 are displayed in FIG. 15. The fusion protein HSA/abrogen secreted from the plasmid pMB059 has the sequence as set forth in SEQ ID NO: 14.

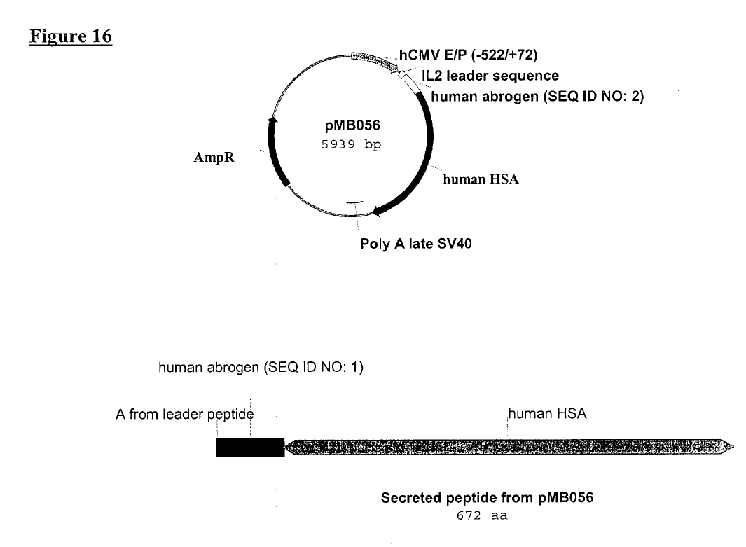

A nucleotide fragment containing from 5′ to 3′ the IL-2 signal peptide, the abrogen nucleotide sequence as set forth in SEQ ID NO: 2, and the sequence of the human HSA (SEQ ID NO: 11), was cloned in pXL2996 downstream to the human CMV promoter and upstream of a SV40 polyA. The resulting plasmid is designated pMB056 and construct are displayed in FIG. 16. The fusion protein HSA/abrogen secreted from the plasmid pMB056 has the sequence as set forth in SEQ ID NO: 15.

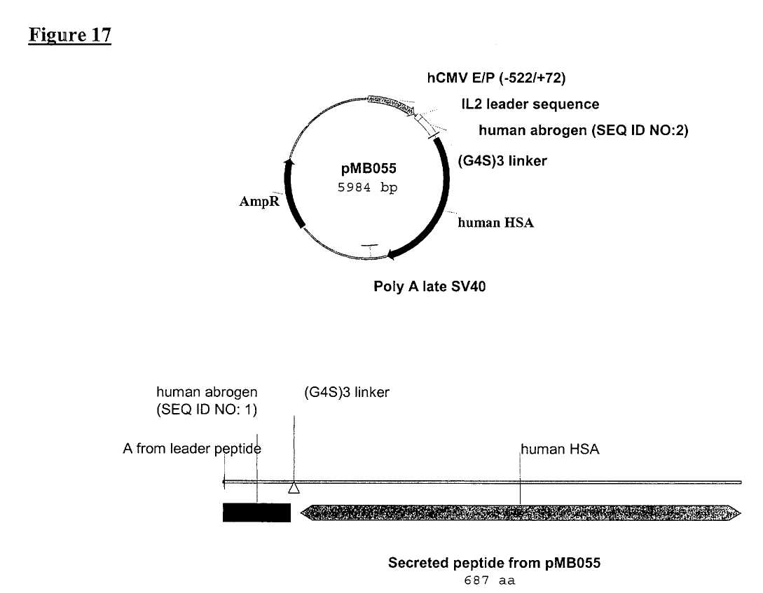

A nucleotide fragment containing from 5′ to 3′ the IL-2 signal peptide, the abrogen nucleotide sequence having the sequence as set forth in SEQ ID NO: 2, a (G₄S)₃ linker (as set forth in SEQ ID NO: 16) and the sequence of the human HSA, was cloned downstream to the human CMV promoter and upstream of a SV40 polyA. The chimeric construct of the fusion protein IL2sp-abrogen-linker-HSA and the resulting plasmid designated pMB055 are displayed in FIG. 17. The fusion protein abrogen/HSA secreted from the plasmid pMB055 has the sequence as set forth in SEQ ID NO: 17.

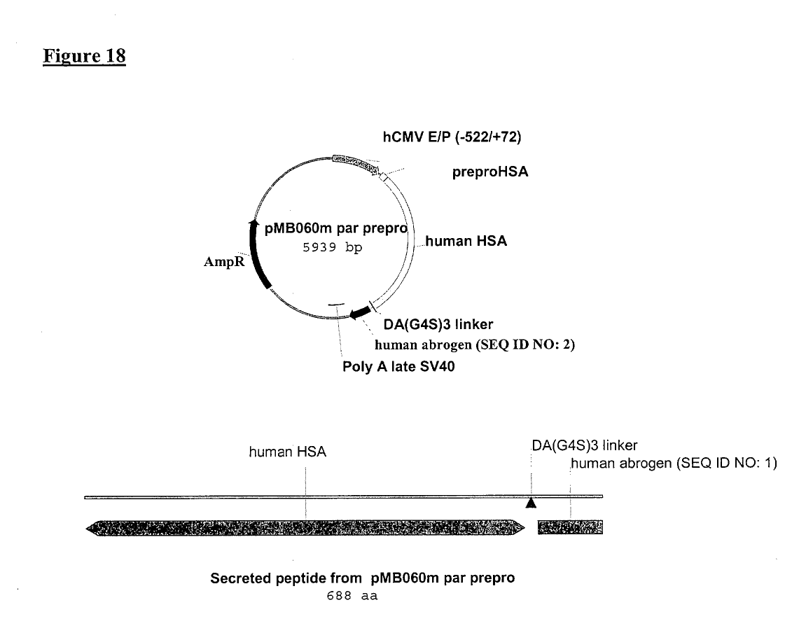

Alternatively, a nucleotide sequence containing from 5′ to 3′ the prepro signal of HSA, the human HSA, a sequence encoding a DA(G₄S)₃ linker and the abrogen nucleotide sequence as set forth in SEQ ID NO: 2 was cloned in the plasmid pXL2996 downstream to the human CMV promoter and upstream of a SV40 polyA. The resulting plasmid is designated pMB060m and the fusion protein prepro HSA-human HSA-DA(G₄S)₃ linker-abrogen are displayed in FIG. 18. The fusion protein HSA/abrogen secreted from the plasmid pMB060m has the sequence as set forth in SEQ ID NO: 18.

A fusion protein encoding plasmid may also comprise the bacteriophage T7 promoter suitable for the production of the kringle polypeptide in E. coli. Such plasmids are also described in U.S. Pat. No. 6,143,518. The plasmid pYG404 as described in the Patent application EP 361 991, which comprise the sequence encoding the prepro-HSA gene, may be used. For example, the C-terminal of HSA is coupled in phase with a linker sequence and the kringle polypeptide nucleotide sequence. The resulting plasmid can also be used for production of the polypeptide in yeasts, for example.

### Example 10

### Construction of Fusion Proteins of Abrogen and IgG2a

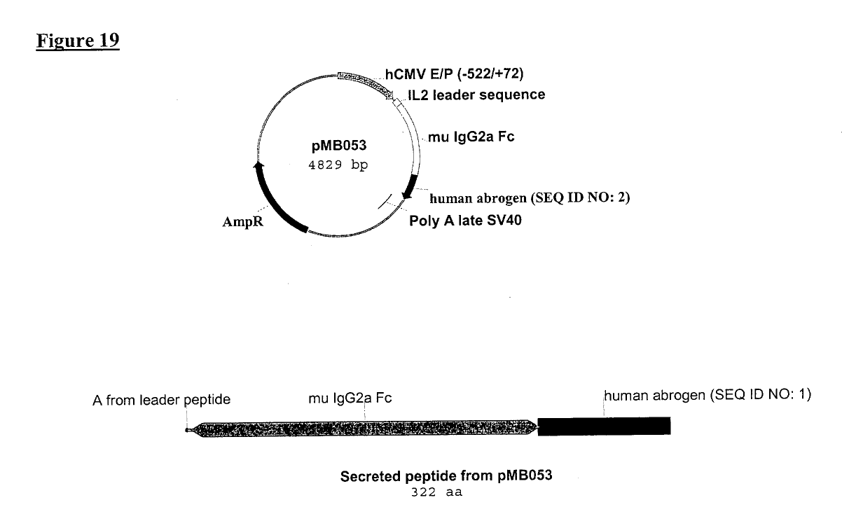

A nucleotide fragment containing from 5′ to 3′ the IL-2 signal peptide, the murin IgG2a Fe region (SEQ ID NO: 19) and the human abrogen nucleotide sequence having the sequence as set forth in SEQ ID NO: 2 was cloned in pXL2996 downstream to the human CMV promoter and upstream of a SV40 polyA. The resulting plasmid is designated pMB053 and the fusion construct are displayed in FIG. 19. The fusion protein IgG2a/abrogen secreted from the plasmid pMB053 has the sequence as set forth in SEQ ID NO: 20.

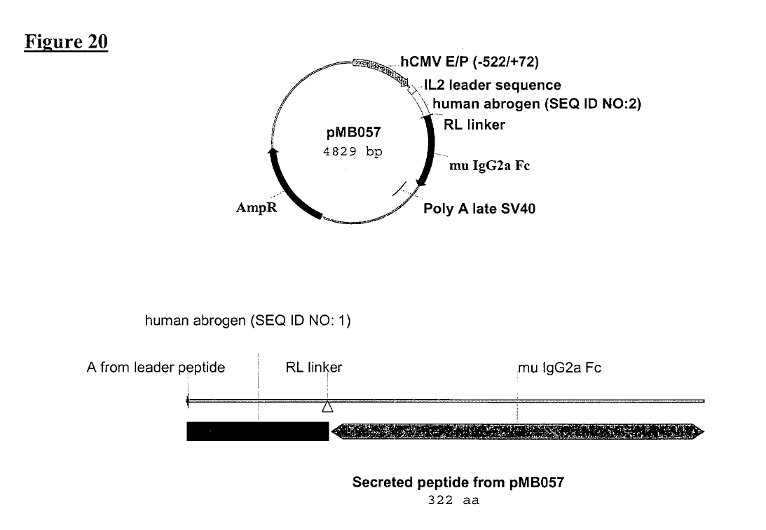

A nucleotide fragment containing from 5′ to 3′ the IL-2 signal peptide, the human abrogen nucleotide sequence having the sequence as set forth in SEQ ID NO: 2, the nucleotide sequence coding for a RL (Arginine-Leucine) linker, the murin (mu) IgG2a Fc region was cloned in pXL2996 downstream to the human CMV promoter and upstream of a SV40 polyA. The resulting plasmid is designated pMB057 and the fusion construct are shown in FIG. 20. The fusion protein abrogen/IgG2a secreted from the plasmid pMB057 has the sequence as set forth in SEQ ID NO: 21.

### Example 11

### Construction of Fusion Protein Construct of trxA and Abrogen Polypeptide

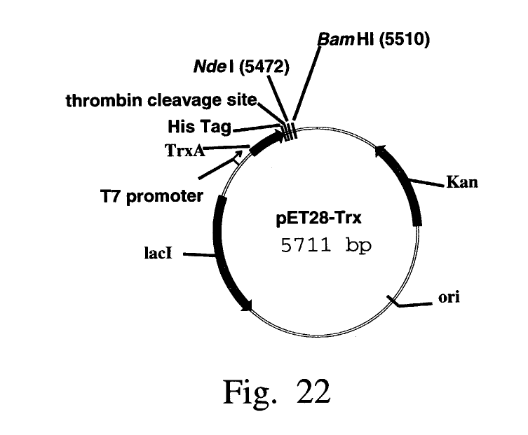

An abrogen polypeptide sequence (SEQ ID NO: 1-14), such as abrogen N43, abrogen D43, K4 from angiostatin (SEQ ID NO: 44), or K5 from plasminogen (SEQ ID NO: 45) as displayed below, can be selected for incorporation into a fusion protein. The kringle polypeptide can then be expressed in soluble form, or substantially soluble form, in E. coli cells with the use of a bacterial expression vector, such as pET28-Trx (see FIG. 22).

The sequences are amplified by PCR and the amplified fragments digested by NdeI-BamHI and cloned into pET28-Trx digested with NdeI-BamHI. Alternatively, sequences can be prepared using synthetic methods or a combination of synthetic and other methods, such as PCR or recombinant manipulation. The following Table presents the sequences selected and the primers used for cloning in an exemplary expression method. The plasmids obtained for the expression of kringle 5 and kringle 4 are also listed in the Table. Templates for the kringle sequences are available from a number of sources.

| | | |
|---|---|---|
| Sequence | Primers | Plasmid for expression |

| | | | |
|---|---|---|---|
| K4 from | Sens: | AAAAGCTTCATATGGCCCAGGACTGCTA | pXL4 190 |
| angiostatin | Antisens: | AAATCTAGAGGATCCTTATCCTGAGCA | |
| K5 from | Sens: | AACATATGGAAGAAGACTGTATGTTTGGGAA | pXL4219 |
| plasminogen | Antisens: | CCGGATCCTTAGGCCGCA | |

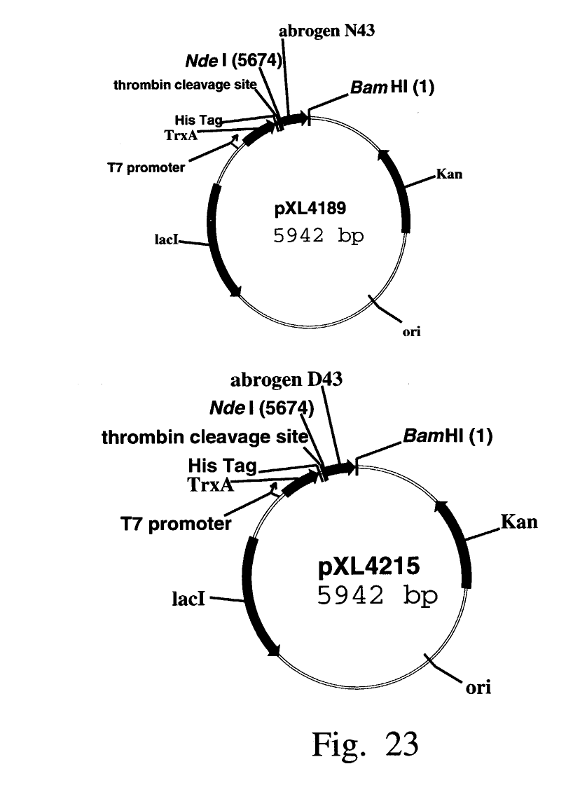

The plasmids for expression are also described in FIGS. 22-23. These plasmids can be sequenced to verify that they encode the expected protein. Exemplary fusion proteins are represented below and comprise a TrxA sequence (from amino acid 2 to 110; see Hoog et al., Biosci. Rep. 4:917 (1984)), a poly-histidine sequence (amino acids 118 to 123), a thrombin cleavage site (amino acids 127-132), followed by the an abrogen kringle peptide.

TrxA-kringle from factor XII
GSDKIIHLTDDSFDTDVLKADGAILVDFWAEWCGPCKMIAPTLDETADEY
QGKLTVAKLNIDQNPGTAPK YGIRGIPTLLLFKNGEVAATKVGALSKGQ
LKEFLDANLAGSGSMGSSHHHHHHSSGLVPRGS
Abrogen kringle from factor XII
GSASCYDGRGLSYRGLARTTLSGAPCQPWASEATYRNVTAEQARNWGLGG
HAFCRNPDNDIRPWCFVLNRDRLSWEYCDLAQCQT
TrxA-kringle from the HGFA
GSDKIIHLTDDSFDTDVLKADGAILVDFWAEWCGPCKNIAPILDEIADEY
QGKLTVAKLNIDQNPGTAPK YGIRGIPTLLLFKNGEVAATKVGALSKGQ
LKEFLDANLAGSGSMGSSHHHHHHSSGLVPRGSERCFLGNGTGYRGVAST
SASGLSCLAWNSDLLYQELHVDSVGAAALLGLGPHAYCRMPDNDERPWCY
VVKDSALSWEYCRLEACES
Abrogen kringle from HGFA
GSERCFLGNGTGYRGVASTSASGLSCLAWNSDLLYQELHVDSVGAAALLGL
GPHAYCRNPDNDERPWCYVVKDSALSWEYCRLEACES
TrxA-kringle from hyaluronan binding protein
GSDKIIHLTDDSFDTDVLKADGAILVDFWAEWCGPCKMIAPILDETADEY
QGKLTVAKLNIDQNPGTAPK YGIRGIPTLLLFKNGEVAATKVGALSKGQ
LKEFLDANLAGSGSMGSSHHHHHHSSGLVPRGSDDCYVGDGYSYRGKMNR
TVNQHACLYWNSHLLLQENYNMFMEDAETHGIGEHNFCRNPDADEKPWCF
IKVTNDKVKWEYCDVSACSA
Abrogen kringle from hyaluronan binding protein
GSDDCYVODGYSYRGKMNRTVNQHACLYWNSHLLLQENYNMFMEDAETHG
IGEHNFCRNPDADEKPWCFIKVTNDKVKWEYCDVSACSA
TrxA-kringle from neurotrypsin
GSDKIIHLTDDSFDTDVLKADGAILVDFWAEWCGPCKMIAPILDEIADEY
QGKLTVAKLNIDQNPGTAPK YGIRGIPTLLLFKNGEVAATKVGALSKGQ
LKEFLDANLAGSGSMGSSHHHHHHSSGLVPRGSWGCPAGEPWVSVTDFGA
PCLRWAEVPPFLERSPPASWAQLRGQRHNFCRSPDGAGRPWCFYGDARGK
VDWGYCDCRH
Abrogen kringle from neurotrypsin
GSWGCPAGEPWVSVTDFGAPCLRWAEVPPFLERSPPASWAQLRGQRHNFC
RSPDGAGRPWCFYGDARGKVDWGYCDCRH
TrxA-kringle from the retinoic acid-related receptor ROR-1
GSDKIIHLTDDSFDTDVLKADGAILVDFWAEWCGPCKMIAPILDEIADEY
QGKLTVAKLNIDQNPGTAPK YGIRGIPTLLLFKNGEVAATKVCALSKCQ
LKEFLDANLAGSGSMGSSHHHHHHSSGLVPRGSHKCYNSTGVDYRGTVSV
TKSCRQCQPWNSQYPHTHTFTALRFPELNGGHSYCRNPGNQKEAPWCFTL
DENFKSDLCDIPACDS
Abrogen kringle from the retinoic acid-related receptor ROR-1
GSHKCYNSTGVDYRGTVSVTKSGRQCQPWNSQYPHTHTFTALRFPELNCCH
SYCRNPGNQKEAPWCFTLDENFKSDLCDIPACDS
TrxA-kringle from the retinoic acid-related receptor ROR-2
GSDKIIHLTDDSFDTDVLKADGAITJVDFWAEWCGPCKMIAPILDEIADEY
QGKLTVAKLNIDQNPGTAPK YGIRGIPTLLIJFKNGEVAATKVGALSKGQ
LKEFLDANLAGSGSMGSSHHHHHHSSCLVPRGSQCYNCSGMDYRGTASTT
KSGHQCQPWALQHPHSHHLSSTDFPELGGGHAYCRNPGGQMEGPWCFTQN
KNVRMELCDVPSCSP
Abrogen kringle from the retinoic acid-related orphan receptor ROR-2
GSQCYNGSCMDYRGTASTTKSGHQCQPWALQHPHSHHLSSTDFPELGGGH
AYCRNPGGQMEGPWCFTQNKNVRMELCDVPSCSP
TrxA-kringle from the kremen protein
GSDKIIHLTDDSFDTDVLKADGAILVDFWAEWCGPCKMIAPILDEIADEY
QGKLTVAKLNILDQNPGTAPK YGIRGIPTLLLFKNGEVAATKVGALSKGQ
LKEFLDANLAGSGSMGSSHHHHHHSSGLVPRGSPECFTANGADYRGTQNW
TALQGGKPCLFWNETFQHPYMTLKYPNGEGGLGEHNYCRNPDGDVSPWCY
VAEHEDGVYWKYCEIPACQM
Abrogen kringle from the kremen protein
GSPECFTANGADYRGTQNWTALQGGKPCLFWNETFQHPYNTLKYPNGEGG
LGEHNYCRNPDGDVSPWCYVAEHEDGVYWKYCEIPACQM
TrxA-kringle from t-PALP
GSDKIIHLTDDSFDTDVLKADGAILVDFWAEWCGPCKMIAPILDEIADEY
QGKLTVAKLNIDQNPGTAPK YGIRGIPTLLLFKNGEVAATKVGALSKGQ
LKEFLDANLAGSGSMGSSHHHHHHSSGLVPRGSGGCFWDNGHLYREDQTS
PAPGLRCLNWLDAQSGLASAPVSGAGNHSYCRNPDEDPRGPWCYVSGEAG
VPEKRPCEDLRCPE
Abrogen kringle from t-PALP
GSGGCFWDNGHLYREDQTSPAPGLRCLNWLDAQSGLASAPVSGAGNHSYCR
NPDEDPRGPWCYVSGEAGVPEKRPCEDLRCPE
TrxA-kringle from the RGD receptor KINASE
GSDKIIHLTDDSFDTDVLKADGAILVDFWAEWCGPCKMIAPILDEIADEY
QGKLTVAKLNIDQNPGTAPK YGIRGIPTLLLFKNGEVAATKVGALSKGQ
LKEFLDANLAGSGSMGSSHHHHHHSSGLVPRGSLACSHPFSKSATEHVQG
HLGKKQVPPDLFQPYIEEICQNLRGDVFQKFIESDKFTRFCQWKNVELNI
HLTMNDFSVHRIIGRGGFGEVYGCRK
Abrogen kringle from the RGD receptor KINASE
GSLACSHPFSKSATEHVQGHLGKKQVPPDLFQPYIEEICQNLRGDVFQKF
IESDKFTRFCQWKNVELNIHLTMNDFSVHRIIGRGGFGEVYGCRK
TrxA-kringle from ApoArgC
GSDKIIHLTDDSFDTDVLKADGAILVDFWAEWCGPCKMIAPILDEIADEY
QGKLTVAKLNILDQNPGTAPK YGIRGIPTLLLFKNGEVAATKVGALSKGQ
LKEFLDANLAGSGSMGSSHHHHHHSSGLVPRGSQECYHSNGQSYRGTYFT
TVTGRTCQAWSSMTPHQHSRTPEKYPNDGLISNYCRNPDGSAGPWCYTTD
PNVRWEYCNLTRCSD
Abrogen kringle from ApoArgC
GSQECYHSNGQSYRGTYFTTVTGRTCQAWSSMTPHQHSRTPEKYPNDGLI
SNYCRNPDGSAGPWCYTTDPNVRWEYCNITRCSD
TrxA-kringles 1-4 from the macrophage stimulating protein
GSDKIIHLTDDSFDTDVIJKADGAILVDFWAEWCGPCKMIAPILDEIADEY
QGKLTVAKLNIDQNPGTAPK YGIRGIPTLLLFKNGEVAATKVGALSKGQ
LKEFLDANLAGSGSMGSSHHHHHHSSGLVPRGSGSRTCIMNNGVGYRGTM
ATTVGGLPCQAWSHKFPNDHKYTPTLRNGLEENFCRNPDGDPGGPWCYTT
DPAVRFQSCGIKSCREAACVWGNGEEYRGAVDRTESGRECQRWDLQHPHQ
HPFEPGKFLDQGLDDNYCRNPDGSERPWCYTTDPQIEREFCDLPRCGSVS
CFRGKGEGYRGTANTTTAGVPCQRWDAQIPHQHRFTPEKYAGKDLRENFC
RNPDGSEAPWCFTLRPGMRAAFCYQIRRCTDQDCYHGAGEQYRGTVSKTR
KGVQCQRWSAETPHKPQFTFTSEPHAQLEENFCRNPDGDSHGPWCYTMDP
RTPFDYCALRRCA
Abrogen kringles 1-4 from the macrophage stimulating protein
GSRTCIMNNGVGYRGTMATTVGGLPCQAWSHKFPNDHKYTPTLRNGLEENF
CRNPDGDPGGPWCYTTDPAVRFQSCGIKSCREAACVWGNGEEYRGAVDRTE
SGRECQRWDLQHPHQHPFEPGKFLDQGLDDNYCRNPDGSERPWCYTTDPQI
EREFCDLPRCGSVSCFRCKGEGYRGTANTTTAGVPCQRWDAQIPHQHRFTP
EKYAGKDLRENFCRNPDGSEAPWCFTLRPGMRAAFCYQIRRCTDQDCYHGA
GEQYRGTVSKTRKGVQCQRWSAETPHKPQFTFTSEPHAQLEENFCRNPDGD
SHGPWCYTMDPRTPFDYCALRRCAD
Translation of pXL4190:TrxA-K4 kringle from angiostatin
GSDKIIHLTD DSFDTDVLKA DGAILVDFWA EWCGPCKMIA PILDEIADEY
QGKLTVAKLN IDQNPGTAPK YGIRGIPTLL LFKNGEVAAT KVGALSKGQL
KEFLDANLAG SGSMGSSHHH HHHSSGLVPR GSHMAQDCYH GDGQSYRGTS
STTTTGKKCQ SWSSMTPHRH QKTPENYPNA GLTMNYCRNP DADKGPWCFT
TDPSVRWEYC NLKKCSG
K4 kringle from angiostatin
GSHMAQDCYH GDGQSYRGTS STTTTGKKCQ SWSSMTPHRH QKTPENYPNA
GLTMNYCRNP DADKGPWCFT TDPSVRWEYC NLKKCSG
Translation ofpXL4219:TrxA-K5 kringle from plasminogen
GSDKIIHLTD DSFDTDVLKA DGAILVDFWA EWCGPCKMIA PILDEIADEY
QGKLTVAKLN IDQNPGTAPK YGIRGIPTLL LFKNGEVAAT KVGALSKGQL
KEFLDANLAG SGSMGSSHHH HHHSSGLVPR GS HMEEDCMF GNGKGYRGKR
ATTVTGTPCQ DWAAQEPHRH STFTPETNPR AGLEKNYCRN PDGDVCGPWC
YTTNPRKLYD YCDVPQCAA
K5 kringle from plasminogen
GSHMEEDCMF GNGKGYRGKR ATTVTGTPCQ DWAAQEPHRH SIFTPETNPR
AGLEKNYCRN PDGDVGGPWC YTTNPRKLYD YCDVPQCAA

The plasmids are introduced into bacteria cells, such as E. coli BL21 λDE3trxB⁻. Isolated clones are inoculated in LB media containing kanamycin for selection at 37° C. After dilution, cultures are grown until an OD600 nm reaches 0.6-1.5. Expression of the fusion protein is initiated at 30° C. by adding IPTG to a final concentration of 1 mM, and continues for 3 hours. Cells are pelleted and an aliquote used to extract total protein, or to separate soluble from insoluble fractions. These samples are analyzed after separation on a polyacrylamide gel (Novex 4-12%) and staining with Coomassie Brilliant Blue.

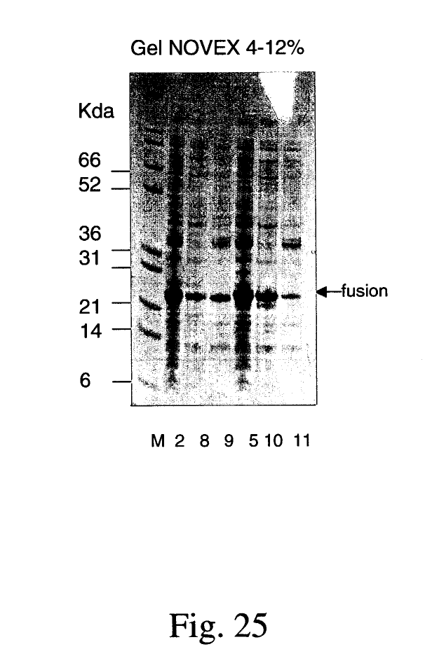

FIG. 25 represents the results obtained with Trx-abrogenN43 and Trx-K4 from angiostatin. The results show that the proteins are expressed at the appropriate molecular weight (around 24 kD) and that they are soluble (around 50% for TrxAbrogenN43 and 90% for Trx-K4). Similar results were obtained with TrxabrogenD43 and Trx-K5 from plasminogen.

### Example 12

### Purification of Abrogen from a Fusion Protein

The kringle polypeptide can be liberated from the fusion protein using a cleavage site present in the fusion protein sequence and an appropriate cleavage enzyme. A variety of cleavage sites and related methods for cleaving a protein are available, including chemical cleavage and terminal peptidases. This example employs the thrombin cleavage site. A cell pellet of 25 grams (centrifugation pellet) from the E. coli BL21 λDE3trxB⁻(pXL4215) cells are taken up with 100 ml of 20 mM potassium phosphate (pH 7.4)-0.5 M NaCl (buffer A), containing 12,500 units of Benzonase™, 35 mg of lysozyme, 0.1% Triton X-100 and 0.5 mM EDTA. The suspension thereby obtained is incubated for 30 min at 37° C., and then centrifuged at 12,000×g for 60 min at +4° C. The supernatant is collected and injected onto a column of Sephadex G-25 (Amersham Biosciences) equilibrated with buffer A and the protein fraction is collected and loaded onto a Hi Trap Chelating HP column (Amersham Biosciences) previously loaded with Ni²⁺ and equilibrated with buffer A containing 10 mM imidazole. The Hi Trap Chelating column is washed with buffer A containing 100 mM imidazole, and the fraction containing fusion protein is eluted with 300 mM imidazole in buffer A. This fraction is chromatographed on a Sephadex G25 column equilibrated with buffer A, collected, mixed with 2 μg of thrombin per mg of protein, and incubated for 16 h at 25° C. The resulting solution is injected onto a Hi Trap Benzamidine Sepharose Fast Flow column (Amersham Biosciences), equilibrated, and eluted with buffer A. The fraction that is not retained on the column is collected and loaded onto a second Hi Trap Chelating HP column previously loaded with Ni²⁺ and equilibrated with buffer A. The liberated kringle polypeptide is eluted from the column with a linear gradient of 0 to 150 mM imidazole in buffer A over 10 column volumes. Purified kringle polypeptide is buffer exchanged by gel filtration on a column of Sephadex G25 equilibrated with PBS (pH 7.4), filtered through a 0.2 μm filter and stored at +4° C. until use.

After this step, the kringle polypeptide is substantially purified. Gel electrophoresis analysis shows a single band by SDS-PAGE after Coomassie staining, centered at a molecular weight estimated at around 10,000. It is unambiguously identified by N-terminal sequencing (10 amino-acids). Protein concentration is quantitated by Coomassie Blue staining with the Bradford reagent.

Typical purification of kringle polypeptide from E. coli BL21 kDE3trxB⁻ (pXL4215)

| | Volume | |
|---|---|---|
| Step | (mL) | Total protein (mg) |
| Crude lysate | 102 | 1020 |
| First Hi Trap Chelating HP | 20 | 113 |
| column eluate | | |
| Hi Trap benzamidine column | 110 | 95 |
| eluate | | |
| Second Hi Trap Chelating HP | 8.0 | 34 |
| column eluate | | |

These data demonstrate the successful production of soluble kringle fusion protein, in an advantageously high percentage compared to prior methods, and the successful generation of biologically active kringle polypeptide from this fusion protein.

### REFERENCES

The references cited below may be referred to above by the reference number. Each of the references is specifically incorporate herein by reference and any part of these references of any reference listed in the text can be relied on to make and use aspects of this invention.

1. Andreasen, P. A., et al., The urokinase-type plasminogen activator system in cancer metastasis: a review. Int J Cancer, 1997. 72(1): p. 1-22.

2. Mukhina, S., et al., The chemotactic action of urokinase on smooth muscle cells is dependent on its kringle domain. Characterization of interactions and contribution to chemotaxis. J Biol Chem, 2000. 275(22): p. 16450-8.

3. Rabbani, S. A., et al., Structural requirements for the growth factor activity of the amino-terminal domain of urokinase. J Biol Chem, 1992. 267(20): p. 14151-6.

4. Quax, P. H., et al., Binding of human urokinase-type plasminogen activator to its receptor: residues involved in species specificity and binding. Arterioscler Thromb Vasc Biol, 1998. 18(5): p. 693-701.

5. Min, H. Y., et al., Urokinase receptor antagonists inhibit angiogenesis and primary tumor growth in syngeneic mice. Cancer Res, 1996. 56(10): p. 2428-33.

6. Li, H., et al., Systemic delivery of antiangiogenic adenovirus AdmATF induces liver resistance to metastasis and prolongs survival of mice. Hum Gene Ther, 1999. 10(18): p. 3045-53.

7. Tang, H., et al., The urokinase-type plasminogen activator receptor mediates tyrosine phosphorylation of focal adhesion proteins and activation of mitogenactivated protein kinase in cultured endothelial cells. J Biol Chem, 1998. 273(29): p. 18268-72.

8. Soff, G. A., Angiostatin and angiostatin-related proteins. Cancer Metastasis Rev, 2000. 19(1-2): p. 97-107.

9. Kleiner, D. E., Jr. and W. G. Stetler-Stevenson, Structural biochemistry and activation of matrix metalloproteases. Curr Opin Cell Biol, 1993. 5(5): p. 891-7.

10. Aguirre Ghiso, J. A., et al., Deregulation of the signaling pathways controlling urokinase production. Its relationship with the invasive phenotype. Eur J Biochem, 1999. 263(2): p. 295-304.

11. Dong, Z., et al., Macrophage-derived metalloelastase is responsible for the generation of angiostatin in Lewis lung carcinoma. Cell, 1997. 88(6): p. 801-10.

12. Cao, Y., et al., Kringle domains of human angiostatin. Characterization of the anti-proliferative activity on endothelial cells. J Biol Chem, 1996. 271(46): p. 29461-7.

13. Cao, Y., et al., Kringle 5 of plasminogen is a novel inhibitor of endothelial cell growth. J Biol Chem, 1997. 272(36): p. 22924-8.

14. Nesbit, M., Abrogation of tumor vasculature using gene therapy. Cancer Metastasis Rev, 2000. 19(1-2): p. 45-9.

15. Lee, T. H., T. Rhim, and S. S. Kim, Prothrombin kringle-2 domain has a growth inhibitory activity against basic fibroblast growth factor-stimulated capillary endothelial cells. J Biol Chem, 1998. 273(44): p. 28805-12.

16. Rhim, T. Y., et al., Human prothrombin fragment 1 and 2 inhibit bFGF-induced BCE cell growth. Biochem Biophys Res Commun, 1998. 252(2): p. 513-6.

17. Xin, L., et al., Kringle 1 of human hepatocyte growth factor inhibits bovine aortic endothelial cell proliferation stimulated by basic fibroblast growth factor and causes cell apoptosis. Biochem Biophys Res Commun, 2000. 277(1): p. 186-90.

18. Chen, C. T., et al., Antiangiogenic gene therapy for cancer via systemic administration of adenoviral vectors expressing secretable endostatin. Hum Gene Ther, 2000.11(14): p. 1983-96.

The additional references below are also specifically incorporated herein by reference.

Lee T-H, Rhim T, Kim SS. Prothrombin kringle-2 domain has a growth inhibitory activity against basic fibroblast growth factor-stimulated capillary endothelial cells. J Biol Chem 1998; 273(44): 28805-28812.

Lu H, Dhanabal M, Volk R, Waterman M J F, Ramchandran R, Knebelmann B, Segal M, Sukhatme VP. Kringle 5 causes cell cycle arrest and apoptosis of endothelial cells. Biochem. Biophys. Res. Corn. 1999; 258: 668-673.

Cao Y, Chen A, Seong Soo A A, Richard-Weidong J, Davidson D, Cao Y, Llinas M. Kringle 5 of plasminogen is a novel inhibitor of endothelial cell growth. J Biol Chem 1997; 272(36): 22924-22928.

Sauter B V, Martinet O, Zhang W-J, Mandeli J, Woo S L C. Adenovirus-mediated gene transfer of endostatin in vivo results in high level of transgene expression and inhibition of tumor growth and metastases. Proc. Natl. Acad. Sci. 2000; 97(9): 48024807.

Li H, Lu H, Griscelli F, Opolon P, Sun L-Q, Ragot T, Legrand Y, Belin D, Soria J, Soria C, Perricaudet M, Yeh P. Adenovirus-mediated delivery of a uPA/uPAR antagonist suppresses angiogenesis-dependent tumor growth and dissemination in mice. Gene Therapy 1998; 5: 1105-1113.

Dong Z, Yoneda J, Kumar R, Fidler I J. Angiostatin-mediated suppression of cancer metastases by primary neoplasms engineered to produce granulocyte/macrophage colony-stimulating factor. J. Exp. Med. 1998; 188(4): 755-763.

Cao Y, Ji R W, Davidson D, Schaller J, Marti D, Sohndel S, McCance S G, O'Reilly M S, Llinas M, Folkmann J. Kringle domains of human angiostatin. J. Biol. Chem. 1996; 271(56): 29461-29467.

Mukhina S, Stepanova V, Traktouev D, Poliakov A, Beabealashvilly R, Gursky Y, Minashkin M, Shevelev A, Tkachuk V. The chemotactic action of urokinase on smooth muscle cells is dependent on its kringle domain. J. Biol. Chem. 2000; 275(22): 16450-16458.

Fischer K, Lutz V, Wilhelm O, Schmitt M, Graeff H, Heiss P, Nishiguchi T, Harbeck N, Luther T, Magdolen V, Reuning U. Urokinase induces proliferation of human ovarian cancer cells: characterization of structual elements required for growth factor function. FEBS Lett. 1998; 438(1-2): 101-105.

Koopman J L, Slomp J, de Bart A C, Quax P H, Verheijen J H. Mitogenic effects of urokinse on melanoma cells are independent of high affinity bindng to the urokinase receptor. J. Biol. Chem. 1998; 273(50): 33267-33272.

Rabbani S A, Mazar A P, Bernier S M, Haq M, Bolivar I, Henkin J, Goltzman D. Structural requirements for the growth factor activity of the amino-terminal domain of urokinase. J. Biol. Chem. 1992; 267(20): 14151-14156.

What is claimed is:

1. A kringle polypeptide having an amino acid sequence consisting of one of SEQ ID NO.: 1-14.

2. The polypeptide of claim 1, wherein the polypeptide reduces endothelial growth induced by both bFGF and VEGF.

3. The polypeptide of claim 1, wherein the protein comprises a kringle domain from a human protein selected from the group consisting of factor XII, hepatocyte growth factor activator, hyaluronan binding protein, neurotrypsin, retinoic acid-related receptors 1 and 2 (ROR-1 and ROR-2), the kremen protein, t-PALP, ApoArgC, and macrophage stimulating proteins (MSP), and wherein the polypeptide is in purified form.

4. The polypeptide of claim 1, further comprising a signal sequence.

5. The polypeptide of claim 1, further comprising an affinity purification sequence.

6. The polypeptide of claim 1, wherein the polypeptide reduces tube formation in cultured endothelial cells.

7. The polypeptide of claim 1, wherein the polypeptide contains 1 to about 10 amino acid changes from any one sequence of SEQ ID NO.: 1-14.

8. The polypeptide of claim 1, wherein the polypeptide contains 1 to about 5 amino acid changes from any one sequence of SEQ ID NO.: 1-14.

9. The polypeptide of claim 1, wherein the N-terminus of the polypeptide is coupled to the signal peptide of interleukin 2.

10. The polypeptide of claim 9, wherein the polypeptide is further coupled to a stabilizing molecule at its C-terminus or N-terminus.

11. The polypeptide of claim 10, wherein the stabilizing molecule is HSA or a IgG2a Fc region.

12. The polypeptide of claim 11, wherein the C-terminus of the polypeptide is coupled to the stabilizing molecule via a linker polypeptide.

13. The polypeptide of claim 12, wherein the linker polypeptide has the sequence as set forth in SEQ ID NO: 32 or 36, or comprises the amino acid sequence ARG-LEU, or ASP-ALA.

14. A method of expressing a soluble kringle polypeptide-containing fusion protein comprising providing a vector or nucleic acid encoding a fusion protein that comprises a kringle polypeptide sequence of one of SEQ ID NO: 1-14 and a TrxA thioredoxin sequence, whereby the fusion protein can be expressed in a bacterial cell, inserting the vector or nucleic acid into a bacterial cell to express the fusion protein, and detecting the presence of soluble fusion protein.

15. The method of claim 14, wherein a substantial fraction of the protein is expressed in a soluble form.

16. The method of claim 14, wherein the bacterial cell is an E. coli cell.

17. A method of preparing a kringle polypeptide composition, comprising expressing a fusion protein according to the method of claim 14, wherein the vector or nucleic acid further comprises an enzymatic cleavage site for liberating the kringle polypeptide sequence from the fusion protein, and further comprising incubating the fusion protein with an appropriate cleavage enzyme to generate kringle polypeptide molecules.

18. The method of claim 17, wherein the enzymatic cleavage site is a thrombin cleavage site.

19. The method of claim 17, further comprising adding a pharmaceutically acceptable excipient or carrier.

20. A method for inhibiting angiogenesis in a cell or tissue associated with an angiogenesis related disease or disorder comprising administering to the cell or tissue at least one kringle polypeptide.

21. A method for treating an angiogenesis related disease or disorder comprising administering at least one kringle polypeptide of claim 1.

22. A method for treating an angiogenesis related disease or disorder comprising administering at least one kringle polypeptide obtained by the method of claim 17.

23. The method of claim 21, wherein the disorder is tumor metastasis, diabetic retinopathy, macular degeneration, obesity, rheumatoid arthritis, or psoriasis.

24. The method of claim 22, wherein the disorder is tumor metastasis, diabetic retinopathy, macular degeneration, obesity, rheumatoid
